(19) United States
(12) Patent Application Publication
(10) Pub. No.: US 2025/0376642 A1
(43) Pub. Date: Dec. 11, 2025

(54) ALKANOLAMINE FORMATES FOR ENZYME STABILIZATION IN LIQUID FORMULATIONS

(71) Applicant: BASF SE, Ludwigshafen Am Rhein (DE)

(72) Inventors: Stephan HUEFFER, Ludwigshafen (DE); Darshan PATWARDHAN-HUBER, Ludwigshafen (DE); Roland ETTL, Ludwigshafen (DE); Larissa BARTULI, Ludwigshafen (DE); Susanne WOLWERTZ, Ludwigshafen (DE)

(21) Appl. No.: 18/874,374

(22) PCT Filed: Jun. 30, 2023
(86) PCT No.: PCT/EP2023/067984

§ 371 Date: Dec. 12, 2024

(30) Foreign Application Priority Data
Jul. 15, 2022 (EP) 22185185.0

(51) Int. Cl. C11D 3/386; C11D 7/32; C12N 9/14; C12N 9/96

(52) U.S. Cl. CPC C11D 3/38663; C11D 7/3245; C12N 9/14; C12N 9/96

(57) ABSTRACT

Described herein are enzyme preparations or detergent formulations including alkanolamine formate to stabilize enzymes, in a liquid environment. Also described herein are liquid enzyme preparations including a) 0.5% to 15% by weight of at least one hydrolase (EC 3) and b) 2% to 70% by weight of at least one alkanolamine formate. Also described herein is a liquid detergent formulation including (A) 0.0005% to 0.4% by weight of at least one hydrolase (EC 3), (B) 4% to 20% by weight of an alkanolamine formate and (C) at least 5% of at least one anionic compound.

### TECHNICAL AREA

The present invention relates to the technical field of enzyme stabilization in liquid formulations. Enzymes comprised in liquid formulations (LF), such as liquid enzyme preparations (LEP) or liquid detergent formulations (LDF) need to be stabilized to avoid loss of function. Thus, the present invention provides a compound that has been identified to stabilize enzymes, preferably hydrolases, in a liquid environment, e. g. liquid enzyme preparations (LEP) and/or liquid detergent formulations (LDF). Further provided are methods of preparing such LEP or LDF and their use.

### BACKGROUND

In liquid formulations, enzymes tend to be instable and are prone to loss of activity upon storage. Therefore, there is a continuous need to identify compounds that improve stabilizations of enzymes in liquid formulations, especially hydrolases such as proteases. Usually, liquid enzyme formulations contain a stabilizing system to improve enzyme stability. These enzyme stabilizers often contain expensive enzyme inhibitors, in particular when proteases are present. Therefore, there is a need to identify alternative compounds that improve stabilizations of enzymes in liquid formulations to reduce or supersede the need for expensive enzyme inhibitors, especially when proteases are present.

A particular challenge for enzyme stabilization in LDF are anionic compounds, such as complexing anionic compounds (also called builders) and/or surface anionic compounds (also called anionic surfactants), comprised in LDF that tend to complex salts present in said formulations. However, salts are necessary in the LDF to stabilize enzymes, preferably proteases and/or lipases. In general, the salt content cannot be arbitrarily raised, since, dependent from the type of salt, the saturation concentration of the salt may be achieved without sufficient enzyme stabilization. Commonly used salts are salts selected from salts comprising
(a) a monovalent cation such as Na⁺, K⁺ and NH₄⁺ and
(b) a monovalent organic anion of 1-6 carbons, which is preferably a small monocarboxylic acid of 1-6 carbons such as formate, acetate, propionate or lactate.

Sodium formate is said to increase subtilisin protease stability in LDF in amounts of about 0.1% to 5% by weight relative to the total weight of the detergent formulation. However, at concentrations of about ≥2.5% by weight relative to the total weight of the detergent formulation, sodium formate tends to precipitate in LDF or trigger clouding and phase separation. This is especially true when the water-content of a liquid detergent formulation is below 50% by weight relative to the total weight of the detergent formulation. Thus, further raising the concentration of sodium formate does not further increase subtilisin stability.

Therefore, it was an additional object of this invention, to find a salt, which does not only improve stability of enzymes such as hydrolases, preferably subtilisin protease and/or triacylglycerol lipase in LDF, but that also does not precipitate in LDF, when used in good stabilizing amounts.

### SUMMARY OF THE INVENTION

To address the above, the invention thus provides a compound according to formula (I)

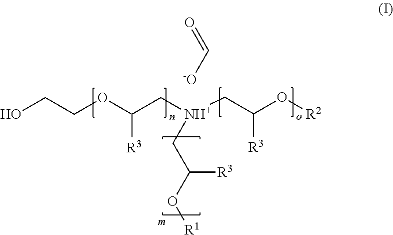

wherein R¹ and R² are selected from H and C₂H₄OH, each of R³ is independently selected from H, methyl and ethyl, preferably all R³ are either H or methyl and m, n, o are each individually 0-2, preferably 0-1, more preferably 0,
that has been identified to stabilize hydrolases in liquid environments, e. g. liquid enzyme preparations (LEP) and/or liquid detergent formulations (LDF).

The compound according to formula (I), alkanolamine formate (AAF) as described herein, has been additionally and surprisingly found to, alone or in combination with salts, preferably with salts at 0.5-2.5% by weight relative to the total weight of the detergent formulation, selected from salts comprising
(a) a monovalent cation such as Na⁺, K⁺ and NH₄⁺ and
(b) a monovalent organic anion of 1-6 carbons, which is preferably a small monocarboxylic acid of 1-6 carbons such as formate, acetate, propionate, or lactate,
increase the stability of enzymes, preferably hydrolases, even more preferably subtilisin protease and/or triacylglycerol lipase in LDF, without increasing the overall salt concentration to saturation concentration within the LDF, thus without precipitating in the LDF when used in good stabilizing amounts.

In a first aspect, the present invention thus refers to liquid enzyme preparations comprising a) 0.5% to 15% by weight of at least one enzyme, preferably hydrolase (EC 3), and b) 2% to 70% by weight of at least one compound according to formula (I) as described herein, wherein the amount of hydrolase refers to 100% active hydrolase.

In a further aspect, the invention provides a liquid detergent formulation comprising (A) 0.0005% to 0.4% by weight of at least one enzyme, preferably hydrolase (EC 3), (B) 4% to 20% by weight of a compound according to formula (I) as described herein and (C) at least 5% of at least one anionic compound.

### DETAILED DESCRIPTION OF THE INVENTION

Liquid formulations (LF), according to the present invention, means products comprising at least one hydrolase (EC 3) and a compound according to formula (I), e.g., liquid enzyme preparations (LEP) or liquid detergent formulations (LDF). According to the invention, liquid formulations contain at least one compound according to formula (I) resulting in stabilization of at least one hydrolase contained.

Thus, the invention, in one embodiment, relates to liquid enzyme preparations (LEP) comprising
a. 0.5% to 15% by weight of at least one enzyme, preferably hydrolase (EC 3), and
b. 2% to 70% by weight of at least one compound according to formula (I)

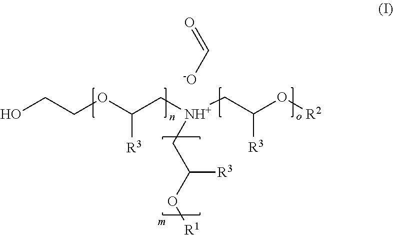

wherein R¹ and R² are selected from H and C₂H₄OH,
each of R³ is independently selected from H, methyl and ethyl, preferably all R³ are either H or methyl, and m, n, o are each individually 0-2, preferably 0-1, more preferably 0;
wherein the amount of hydrolase refers to 100% active hydrolase.
In another aspect, the invention relates to liquid detergent formulations (LDF) comprising:
(A) 0.0005% to 0.4% by weight of at least one enzyme, preferably hydrolase (EC 3)
(B) 4% to 20% by weight of a compound according to formula (I)

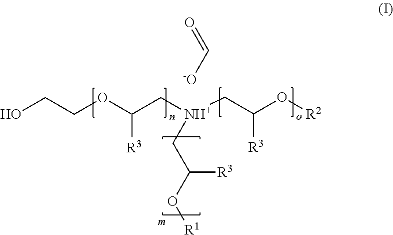

wherein R¹ and R² are selected from H and C₂H₄OH, each of R³ is independently selected from H, methyl and ethyl, preferably all R³ are either H or methyl, and m, n, o are each individually 0-2, preferably 0-1, more preferably 0; and
(C) at least 5% of at least one anionic compound.

### Component a./(A)—Hydrolase (EC 3)

Liquid formulations (LF) of the invention comprise at least one enzyme, preferably a hydrolase (EC 3).

Hydrolases means enzymes exerting enzymatic activity. Enzymatic activity relates to the capability of a hydrolase to degrade respective substrates. The at least one hydrolase preferably originates from fermentative production.

“Fermentative production” means the process of cultivating recombinant cells, which express the desired hydrolase in a suitable water-based nutrient medium, allowing the recombinant host cells to grow and express the desired hydrolase. At the end of the fermentation, the fermentation broth is usually collected, and the liquid fraction is separated from the solid fraction. Depending on whether the hydrolase has been secreted into the liquid fraction or not, the desired hydrolase can be recovered from the liquid fraction of the fermentation broth or from cell lysates. Recovery of the desired hydrolase uses methods known to those skilled in the art. Suitable methods for recovery of hydrolases from fermentation broth include but are not limited to collection, centrifugation, filtration, extraction, and precipitation.

In one embodiment, the liquid formulation contains an “enzyme concentrate”, meaning that the fermentation broth containing the hydrolase has already been purified and concentrated. Liquid enzyme concentrates usually comprise amounts of hydrolase up to 40% by weight or up to 30% by weight or up to 25% by weight, all relative to the total weight of the enzyme concentrate.

Enzyme concentrates which result from fermentation comprise water and potentially further residual components such as salts originating from the fermentation medium, cell debris originating from the production host cells, metabolites produced by the production host cells during fermentation. Residual components may be comprised in liquid enzyme concentrates in amounts less than 20% by weight relative to the total weight of the enzyme concentrate. Preferably residual components are comprised in amounts less than 10% by weight, more preferably less than 5% by weight, all relative to the total weight of the enzyme concentrate. Liquid formulations, in another aspect, contain at least one solid hydrolase (EC 3), which is dissolved in at least one solvent selected from water and organic solvents. Preferably, said liquid formulation comprises amounts of hydrolase below saturation concentration of the hydrolase, meaning that the hydrolase is dissolved in the liquid formulation and no precipitation occurs.

Hydrolases may be parent hydrolases or variants thereof. A “parent hydrolase” or “parent sequence” (of a parent protein or polypeptide) is the starting sequence for introduction of changes (e. g. by introducing one or more amino acid substitutions, insertions, deletions, or a combination thereof) to the sequence, resulting in “variants” of the parent sequences. The term parent enzyme (or parent sequence) includes wild-type enzymes (sequences) and synthetically generated sequences (enzymes), which are used as starting sequences for introduction of (further) changes. The term “hydrolase variant” or “sequence variant” or “variant hydrolase” refers to a hydrolase that differs from a parent hydrolase in its amino acid sequence to a certain extent. If not indicated otherwise, variant enzyme “having enzymatic activity” means that this variant enzyme has the same type of enzymatic activity as the respective parent enzyme.

In describing hydrolase variants, usually substitutions, deletions and insertions occur when compared to a parent sequence. Herein nomenclature is used known to those skilled in the art. Amino acid substitutions are usually described by providing the original amino acid followed by the number of the position within the amino acid sequence, followed by the substituted amino acid. Amino acid deletions are usually described by providing the original amino acid followed by the number of the position within the amino acid sequence, followed by *. Amino acid insertions are usually described by providing the original amino acid followed by the number of the position within the amino acid sequence, followed by the original amino acid and the additional amino acid. Where different alterations can be introduced at a position, the different alterations are separated by a slash.

Hydrolase variants are usually defined by their sequence identity when compared to a parent hydrolase. Sequence identity usually is provided as “% sequence identity” or “% identity”. For calculation of sequence identities, in a first step a sequence alignment has to be produced. According to this invention, a pairwise global alignment has to be produced, meaning that two sequences have to be aligned over their complete length, which is usually produced by using a mathematical approach, called alignment algorithm. According to the invention, the alignment is generated by using the algorithm of Needleman and Wunsch (J. Mol. Biol. (1979) 48, p. 443-453). Preferably, the program “NEEDLE” (The European Molecular Biology Open Software Suite (EMBOSS)) is used for the purposes of the current invention, with using the programs default parameter (gap open=10.0, gap extend=0.5 and matrix=EBLOSUM62). According to this invention, the following calculation of %-identity applies: %-identity=(identical residues/length of the alignment region which is showing the respective sequence of this invention over its complete length)*100.

According to this invention, hydrolase variants are described as an amino acid sequence which is at least n % identical to the amino acid sequence of the respective parent hydrolase with “n” being an integer between 10 and 100. In one embodiment, variant hydrolases are with increasing preference at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical when compared to the full-length amino acid sequence of the parent hydrolase, wherein the enzyme variant has enzymatic activity.

“Enzymatic activity” usually relates to degradation of a hydrolase target substrate. “Enzymatic activity” means the catalytic effect exerted by a hydrolase, which usually is expressed as units per milligram of enzyme (specific activity), which relates to molecules of substrate transformed per minute per molecule of enzyme (molecular activity).

In a preferred embodiment, the hydrolase is selected from proteases, amylases, lipases, cellulases, hemicellulase, mannanases, xylanases, DNases, dispersins, pectinases, and cutinases, preferably selected from subtilisin protease (EC 3.4.21.62), alpha-amylase (EC 3.2.1.1), and triacylglycerol lipase (EC 3.1.1.3).

### Proteases

Proteases (EPr) are members of the enzyme class EC 3.4. Proteases include aminopeptidases (EC 3.4.11, EPr1), dipeptidases (EC 3.4.13, EPr2), dipeptidyl-peptidases and tripeptidyl-peptidases (EC 3.4.14, EPr3), peptidyl-dipeptidases (EC 3.4.15, EPr4), serine-type carboxypeptidases (EC 3.4.16, EPr5), metallocarboxypeptidases (EC 3.4.17, EPr6), cysteine-type carboxypeptidases (EC 3.4.18, EPr7), omega peptidases (EC 3.4.19, EPr8), serine endopeptidases (EC 3.4.21, EPr9), cysteine endopeptidases (EC 3.4.22, EPr10), aspartic endopeptidases (EC 3.4.23, EPr11), metallo-endopeptidases (EC 3.4.24, EPr12), threonine endopeptidases (EC 3.4.25, EPr13), or endopeptidases of unknown catalytic mechanism (EC 3.4.99, EPr14).

Proteases means enzymes exerting proteolytic activity. Proteolytic activity relates to the capability of a protease to degrade proteins.

Proteases may be parent enzymes or variants thereof, wherein parent proteases include wild type proteases as well as starting proteases for further mutations. Variant proteases mean mutated parent proteases. Parent proteases as well as variant proteases have to have proteolytic activity to be proteases according to the disclosure.

Protease variants may have less than, essentially equal than, or increased proteolytic activity when compared to the parent protease. Proteolytic activity of a variant is preferably at least 75%, at least 80%, at least 85%, at least 90%, at least 95%, or at least 100% of the proteolytic activity of the respective parent protease. Increased proteolytic activity of a variant means greater 100%, preferably at least 105%, proteolytic activity when compared to the respective parent protease.

LEP or LDF disclosed herein may comprise at least one protease selected from serine proteases (EC 3.4.21, EPr9). Serine proteases or serine peptidases are characterized by having a serine in the catalytically active site, which forms a covalent adduct with the substrate during the catalytic reaction. LEP or LDF, in one embodiment, comprise at least one EPr9 selected from the group consisting of chymotrypsin (EPr9a; EC 3.4.21.1), caldecrin (EPr9b; EC 3.4.21.2), elastase (EPr9c; EC 3.4.21.36, EC 3.4.21.37, EC 3.4.21.70, EC 3.4.21.71), granzyme (EPr9d; EC 3.4.21.78 or EC 3.4.21.79), kallikrein (EPr9e; EC 3.4.21.34, EC 3.4.21.35, EC 3.4.21.118, EC 3.4.21.119,) plasmin (EPr9f; EC 3.4.21.7), trypsin (EPr9g; EC 3.4.21.4), thrombin (EPr9h, EC 3.4.21.5), and subtilisin (EPr9i). EPr9i is also known as subtilopeptidase, e.g. EC 3.4.21.62, the latter hereinafter also being referred to as “subtilisin”. Subtilisins (EPr9i) and chymotrypsin (EPr9a) are related serine proteases both having a catalytic triad comprising aspartate, histidine, and serine. In EPr9i the relative order of these amino acids, reading from the amino- to the carboxy-terminus is aspartate-histidine-serine. In EPr9a the relative order is histidine-aspartate-serine. A wide variety of EPr9i have been identified, and the amino acid sequence of a number of subtilases has been determined. For a more detailed description of such subtilases and their amino acid sequences reference is made to Siezen et al. (1997), Protein Science 6:501-523.

In one embodiment, LEP or LDF comprise at least one EPr9i, which are bacterial subtilisins. Said bacterial protease may be a Gram-positive bacterial polypeptide such as a Bacillus, Clostridium, Enterococcus, Geobacillus, Lactobacillus, Lactococcus, Oceanobacillus, Staphylococcus, Streptococcus, or Streptomyces protease, or a Gram-negative bacterial polypeptide such as a Campylobacter, Escherichia, Flavobacterium, Fusobacterium, Helicobacter, Ilyobacter, Neisseria, Pseudomonas, Salmonella, or Ureaplasma protease. A review of this family is provided, for example, in “Subtilases: Subtilisin-like Proteases” by R. Siezen, pages 75-95 in “Subtilisin enzymes”, edited by R. Bott and C. Betzel, New York, 1996. In one embodiment, LEP or LDF comprise at least one EPr9i selected from subtilisins originating from Bacillus alcalophilus, Bacillus amyloliquefaciens, Bacillus brevis, Bacillus circulans, Bacillus clausii, Bacillus coagulans, Bacillus firmus, Bacillus gibsonii, Bacillus lautus, Bacillus lentus, Bacillus licheniformis, Bacillus megaterium, Bacillus pumilus, Bacillus sphaericus, Bacillus stearothermophilus, Bacillus subtilis, or Bacillus thuringiensis.

Specifically, at least one EPr9i may be selected from the following: subtilisin from Bacillus amyloliquefaciens BPN′ (described by Vasantha et al. (1984) J. Bacteriol. Volume 159, p. 811-819 and Wells et al. (1983) in Nucleic Acids Research, Volume 11, p. 7911-7925); subtilisin from Bacillus licheniformis (subtilisin Carlsberg; disclosed in Smith et al. (1968) in J. Biol Chem, Volume 243, pp. 2184-2191, and Jacobs et al. (1985) in Nucl. Acids Res, Vol 13, p. 8913-8926); subtilisin PB92 (original sequence of the alkaline protease PB92 is described in EP 283075 A2); subtilisin 147 (Esperase®), subtilisin 309 (Savinase®, see Table I of WO 89/06279) as disclosed in WO 89/06279; subtilisin from Bacillus lentus as disclosed in WO 91/02792, such as from Bacillus lentus DSM 5483 or the variants of Bacillus lentus DSM 5483 as described in WO 95/23221; subtilisin from Bacillus alcalophilus (DSM 11233) disclosed in DE 10064983; subtilisin from Bacillus gibsonii (DSM 14391) as disclosed in WO 2003/054184; subtilisin from Bacillus sp. (DSM 14390) disclosed in WO 2003/056017; subtilisin from Bacillus sp. (DSM 14392) disclosed in WO 2003/055974; subtilisin from Bacillus gibsonii (DSM 14393) disclosed in WO 2003/054184; subtilisin having SEQ ID NO: 4 as described in WO 2005/063974; subtilisin having SEQ ID NO: 4 as described in WO 2005/103244; subtilisin having SEQ ID NO: 7 as described in WO 2005/103244; and subtilisin having SEQ ID NO: 2 as described in application DE 102005028295.4.

Examples of subtilisins comprised in LEP or LDF include but are not limited to the variants described in: WO 92/19729, WO 95/23221, WO 96/34946, WO 98/20115, WO 98/20116, WO 99/11768, WO 01/44452, WO 02/088340, WO 03/006602, WO 2004/03186, WO 2004/041979, WO 2007/006305, WO 2011/036263, WO 2011/036264, and WO 2011/072099.

In one embodiment, LEP or LDF comprise at least one subtilisin which is at least 80% identical to a polypeptide sequence according to SEQ ID NO: 22 as described in EP 1921147 (which is the sequence of mature alkaline protease from Bacillus lentus DSM 5483; the 100% identical sequence may be called BLAP WT herein). Preferably, said subtilisin protease is not mutated at positions Asp32, His64 and Ser221 (according to BPN′ numbering). A subtilisin which is at least 80% identical to a polypeptide sequence according to SEQ ID NO: 22 as described in EP 1921147 may be called EPr9iA herein.

In one embodiment, EPr9iA has at least a substitution at position 101, preferably selected from R101E, R101D and R101S (according to BPN′ numbering).

In one embodiment, EPr9iA has one or more substitutions selected from 3T, 41, 63A/T/R, 156D/E, 194P, 199M, 205I and 217D/E/G, optionally together with a substitution at position 101 selected from R101E, R101D and R101S, wherein the numbering is according to the BPN′ numbering.

In one embodiment, EPr9iA has one or more substitutions selected from S156D, L262E, Q137H, S3T, R45E/D/Q, P55N, T58W,Y,L, Q59D/M/N/T, G61D/R, S87E, G97S, A98D/E/R, S106A/W, N117E, H120V/D/K/N, S125M, P129D, E136Q, S144W, S161T, S163A/G, Y171L, A172S, N185Q, V199M, Y209W, M222Q, N238H, V244T, N261T/D and L262N/Q/D, and optionally a substitution at position 101 selected from R101E, R101D and R101S, and wherein the numbering is according to the BPN′ numbering.

In one embodiment, LF of the invention comprises
(A) at least one serine protease EPr9iA having at least the R101E or R101D or R101S, preferably R101E (according to BPN′ numbering), and
(B) at least one alkanolamine formate according to formula (I) as described herein.

In one embodiment, LEP of the invention comprises
a. at least one serine protease EPr9iA having at least the R101E or R101D or R101S, preferably R101E (according to BPN′ numbering), and
b. at least one alkanolamine formate according to formula (I) as described herein.

In one embodiment, LDF of the invention comprises
(A) at least one serine protease EPr9iA having at least the R101E or R101D or R101S, preferably R101E (according to BPN′ numbering), and
(B) at least one alkanolamine formate according to formula (I) as described herein, and
(C) at least 5% of at least one anionic compound.

In one embodiment, component a./(A) comprises at least one EPr9iA having one or more substitutions selected from 3T, 41, 63A/T/R, 156D/E, 194P, 199M, 205I and 217D/E/G, and optionally further having a substitution R101E or R101D or R101S, wherein the numbering is according to the BPN′ numbering.

In one embodiment, component a./(A) comprise at least one EPr9iA having one or more substitutions selected from S156D, L262E, Q137H, S3T, R45E/D/Q, P55N, T58W/Y/L, Q59D/M/N/T, G61D/R, S87E, G97S, A98D,E,R, S106A/W, N117E, H120V/D/K/N, S125M, P129D, E136Q, S144W, S161T, S163A/G, Y171L, A172S, N185Q, V199M, Y209W, M222Q, N238H, V244T, N261T/D and L262N/Q/D, and optionally further having a substitution R101E or R101D or R101S, wherein the numbering is according to the BPN′ numbering.

In one embodiment, component a./(A) comprises at least one EPr9iA having mutations selected from S3T+V4I+V205I, S3T+V4I+R101E+V205I and S3T+V4I+V199M+V205I+L217D (according to BPN′ numbering).

In one embodiment, component a./(A) comprises at least one EPr9iA having mutations S3T+V4I+S9R+A15T+V68A+D99S+R101S+A103S+I104V+N218D (according to BPN′ numbering).

EPr9, preferably EPr9i, more preferably EPr9iA may be stabilized by at least one enzyme stabilizer selected from boron-containing stabilizers and peptide stabilizers.

In one embodiment, LEP or LDF comprise therefore in addition to at least one protease at least one boron-containing stabilizer (PSB) selected from
(a) boric acid or its derivatives,
(b) boronic acid or its derivatives such as aryl boronic acids or its derivatives,
(c) salts of (a) or (b), and
(d) mixtures thereof.

Boric acid herein may be called orthoboric acid. In one embodiment, the boron-containing stabilizer is selected from the group consisting of benzene boronic acid (BBA) which is also called phenyl boronic acid (PBA), derivatives thereof, and mixtures thereof.

In one embodiment, at least one phenyl-boronic acid derivative is selected from 4-formyl phenyl boronic acid (4-FPBA, PSB1), 4-carboxy phenyl boronic acid (4-CPBA, PSB2), 4-(hydroxymethyl) phenyl boronic acid (4-HMPBA, PSB3) and p-tolylboronic acid (p-TBA, PSB4), with PSB1 being preferred.

In one embodiment, LEP or LDF comprise therefore in addition to at least one protease at least one enzyme stabilizer that is a peptide stabilizer (PSP), preferably selected from tri-peptide compounds comprising three amino acids selected from glycine, valine, alanine, tyrosine and leucine. The tri-peptide stabilizer is preferably selected from peptide aldehydes, peptide acetals, and peptide aldehyde hydrosulfite adducts. Usually, tri-peptide stabilizers carry an N-terminal protection group. Preferably, the tri-peptide stabilizer is selected from a compound comprising Glycine-Alanine-Tyrosine (GAY, PSP1, preferably Z-GAY-H) and Valine-Alanine-Leucine (VAL, PSP2, preferably Z-VAL-H) in combination with an N-terminal protection group such as benzyloxycarbonyl (Cbz). A tripeptide stabilizer VAL with the CbZ protection group may be called Z-VAL herein.

In another embodiment, LEP or LDF comprise in addition to at least one protease at least one enzyme stabilizer that is a peptide stabilizer (PSP) and at least one stabilizer that is a boron-containing stabilizer.

Specifically, LDF may comprise one of the following combinations (C-PrPS):

| id | actual combinations |
|---|---|
| C-PrPS1 | EPr9iA + PSB1 |
| C-PrPS2 | EPr9iA + PSB2 |
| C-PrPS3 | EPr9iA + PSB3 |
| C-PrPS4 | EPr9iA + PSB4 |
| C-PrPS5 | EPr9iA + PSP1 |
| C-PrPS6 | EPr9iA + PSP2 |

whereas preferably, the peptide stabilizer is a peptide aldehyde, peptide aldehyde hydrosulfite adduct, or peptide acetal, preferably a peptide aldehyde, most preferably Z-GAY-H or ZVAL-H. Preferably, EPr9iA in C-PrPS1 to C-PrPS6 is EPr9iA having at least a substitution at position 101, preferably selected from R101E, R101D and R101S, preferably R101E. In one embodiment, PSP2 in C-PrPS6 means Z-VAL, preferably Z-VAL-H.

In one embodiment, LEP or LDF comprise more than one protease. Specifically, one of the following combinations may be comprised:

| identifier | combination |
|---|---|
| C-Pr1 | EPr9a + EPr9b |
| C-Pr2 | EPr9a + EPr9c |
| C-Pr3 | EPr9a + EPr9d |
| C-Pr4 | EPr9a + EPr9e |
| C-Pr5 | EPr9a + EPr9f |
| C-Pr6 | EPr9a + EPr9g |
| C-Pr7 | EPr9a + EPr9h |
| C-Pr8 | EPr9a + EPr9i |
| C-Pr9 | EPr9a + EPr9iA |
| C-Pr10 | EPr9b + EPr9c |
| C-Pr11 | EPr9b + EPr9d |
| C-Pr12 | EPr9b + EPr9e |
| C-Pr13 | EPr9b + EPr9f |
| C-Pr14 | EPr9b + EPr9g |
| C-Pr15 | EPr9b + EPr9h |
| C-Pr16 | EPr9b + EPr9i |
| C-Pr17 | EPr9b + EPr9iA |
| C-Pr18 | EPr9c + EPr9d |
| C-Pr19 | EPr9c + EPr9e |
| C-Pr20 | EPr9c + EPr9f |
| C-Pr21 | EPr9c + EPr9g |
| C-Pr22 | EPr9c + EPr9h |
| C-Pr23 | EPr9c + EPr9i |
| C-Pr24 | EPr9c + EPr9iA |
| C-Pr25 | EPr9d + EPr9e |
| C-Pr26 | EPr9d + EPr9f |
| C-Pr27 | EPr9d + EPr9g |
| C-Pr28 | EPr9d + EPr9h |
| C-Pr29 | EPr9d + EPr9i |
| C-Pr30 | EPr9d + EPr9iA |
| C-Pr31 | EPr9e + EPr9f |
| C-Pr32 | EPr9e + EPr9g |
| C-Pr33 | EPr9e + EPr9h |
| C-Pr34 | EPr9e + EPr9i |
| C-Pr35 | EPr9e + EPr9iA |
| C-Pr36 | EPr9f + EPr9g |
| C-Pr37 | EPr9f + EPr9h |
| C-Pr38 | EPr9f + EPr9i |
| C-Pr39 | EPr9f + EPr9iA |
| C-Pr40 | EPr9g + EPr9h |
| C-Pr41 | EPr9g + EPr9i |
| C-Pr42 | EPr9g + EPr9iA |
| C-Pr43 | EPr9h + EPr9i |
| C-Pr44 | EPr9h + EPr9iA |

### Amylases

Amylases means enzymes exerting “amylolytic activity” or “amylase activity”. Amylolytic activity relates to the capability of an amylase to hydrolyze glycosidic linkages in polysaccharides, preferably at the endo-position. Amylases may be parent amylases or variants thereof. Parent amylases as well as variant amylases have to have amylolytic activity to be amylases according to the invention.

LEP or LDF, in one embodiment, comprise at least one alpha-amylases (EC 3.2.1.1; Amya).

Preferably, LEP or LDF comprise at least one Amya selected from:
Amylases from Bacillus licheniformis having SEQ ID NO: 2 as described in WO 95/10603 and variants at least 95% identical thereto (Amyα1). Suitable variants are described in WO 95/10603 comprising one or more substitutions in the following positions: 15, 23, 105, 106, 124, 128, 133, 154, 156, 178, 179, 181, 188, 190, 197, 201, 202, 207, 208, 209, 211, 243, 264, 304, 305, 391, 408, and 444, which have amylolytic activity. Variants are described in WO 94/02597, WO 94/018314, WO 97/043424 and SEQ ID NO: 4 of WO 99/019467.
Amylases from B. stearothermophilus having SEQ ID NO: 6 as disclosed in WO 02/10355 and variants at least 95% identical thereto (Amyα2). Said amylase may be truncated at the C-terminus (Amyα2a). Suitable variants of SEQ ID NO: 6 include those comprising a deletion at positions 181 and/or 182 and/or a substitution at position 193.
Amylases from Bacillus sp. 707 having SEQ ID NO: 6 as disclosed in WO 99/19467 and variants at least 95% identical thereto (Amyα3). Preferred variants of SEQ NO: 6 are those having a substitution, a deletion or an insertion at one or more of the following positions: R181, G182, H183, G184, N195, I206, E212, E216, and K269.
Amylases from Bacillus halmapalus having SEQ ID NO: 2 or SEQ ID NO: 7 as described in WO 96/23872, also described herein as SP-722 (Amyα4). Preferred variants are described in WO 97/3296, WO 99/194671 and WO 2013/001078.
Amylases from Bacillus sp. DSM 12649 having SEQ ID NO: 4 as disclosed in WO 00/22103 and variants at least 95% identical thereto (Amyα5).
Amylases from Bacillus sp. A 7-7 (DSM 12368) having an amino acid sequence at least 95% identical to SEQ ID NO: 2 (Amyα6), in particular over the region of the amino acids 32 to 516 according to SEQ ID NO: 2, as disclosed in WO 02/10356.

Amylases from the Bacillus strain TS-23 having SEQ ID NO: 2 as disclosed in WO 2009/061380 and variants at least 95% identical thereto (Amyα7).
Amylases from Cytophaga sp. having SEQ ID NO: 1 as disclosed in WO 2013/184577 and variants at least 95% identical thereto (Amyα8).
Amylases from Bacillus megaterium DSM 90 having SEQ ID NO: 1 as disclosed in WO 2010/104675 and variants at least 95% identical thereto (Amyα9).
Amylases from Bacillus sp. comprising amino acids 1 to 485 of SEQ ID NO: 2 as described in WO 00/60060 and variants at least 95% identical thereto (Amyα10).
Amylases from Bacillus amyloliquefaciens or variants at least 95% identical thereto, preferably selected from amylases according to SEQ ID NO: 3 as described in WO 2016/092009 (Amyα11).
Amylases having SEQ ID NO: 12 as described in WO 2006/002643 or amylase variants at least 95% identical thereto (Amyα12), preferably comprising the substitutions Y295F and M202L/I/T/V within said SEQ ID NO: 12.
Amylases having SEQ ID NO: 6 as described in WO 2011/098531 or amylase variants at least 95% identical thereto (Amyα13), preferably comprising one or more substitutions at positions selected from 193G/A/S/T/M, 195F/W/Y/L/I/V, 197F/W/Y/L/I/V, 198Q/N, 200F/W/Y/L/I/V, 203F/W/Y/L/I/V, 206F/W/Y/N/L/I/V/H/Q/D/E, 210F/W/Y/L/I/V, 212F/W/Y/L/I/V, 213G/A/S/T/M and 243F/W/Y/L/I/V within said SEQ ID NO: 6.
Amylases having SEQ ID NO: 1 as described in WO 2013/001078 or amylase variants at least 95% identical thereto (Amyα14), preferably comprising an alteration at two or more (several) positions corresponding to positions G304, W140, W189, D134, E260, F262, W284, W347, W439, W469, G476, and G477 within said SEQ ID NO: 1.
Amylases having SEQ ID NO: 2 as described in WO 2013/001087 or amylase variants at least 95% identical thereto (Amyα15), preferably comprising a deletion of positions 181+182 or 182+183 or 183+184 within said SEQ ID NO: 2; optionally said sequence comprises one or two or more modifications in any position selected from W140, W159, W167, Q169, W189, E194, N260, F262, W284, F289, G304, G305, R320, W347, W439, W469, G476 and G477 within said SEQ ID NO: 2.
Amylases which are hybrid alpha-amylases from above mentioned amylases as for example described in WO 2006/066594 (Amyα16).
Hybrid amylases according to WO 2014/183920 with A and B domains having at least 90% identity to SEQ ID NO: 2 of WO 2014/183920 and a C domain having at least 90% identity to SEQ ID NO: 6 of WO 2014/183920; preferably the hybrid alpha-amylase is at least 95% identical to SEQ ID NO: 23 of WO 2014/183920 (Amyα17).
Hybrid amylase according to WO 2014/183921 with A and B domains having at least 75% identity to SEQ ID NO: 2, SEQ ID NO: 15, SEQ ID NO: 20, SEQ ID NO: 23, SEQ ID NO: 29, SEQ ID NO: 26, SEQ ID NO: 32, and SEQ ID NO: 39 as disclosed in WO 2014/183921 and a C domain having at least 90% identity to SEQ ID NO: 6 of WO 2014/183921; preferably, the hybrid alpha-amylase is at least 95% identical to SEQ ID NO: 30 as disclosed in WO 2014/183921 (Amyα18).
Hybrid amylase according to WO 2021/032881 comprising an A and B domain originating from the alpha amylase from Bacillus sp. A 7-7 (DSM 12368) and a C domain originating from the alpha amylase from Bacillus cereus wherein the hybrid amylase has amylolytic activity; preferably, the A and B domain are at least 95% identical to the amino acid sequence of SEQ ID NO: 42 and a C domain is at least 95% identical to the amino acid sequence of SEQ ID NO: 44, both sequences as disclosed in WO 2021/032881; more preferably, the hybrid amylase is SEQ ID NO: 54 as disclosed in WO 2021/032881 (Amyα19).

In one embodiment, LEP or LDF comprise more than one Amyα. Preferably, LEP comprise two or more Amya selected from Amyα3, Amyα6, Amyα10, Amyα13, Amyα14, Amyα15, Amyα17, Amyα18 and Amyα19.

In one embodiment, LEP or LDF comprise more than one amylase. Specifically, one of the following combinations may be comprised:

| identifier | combination |
|---|---|
| C-Amyα1 | Amyα1 + Amyα2 |
| C-Amyα2 | Amyα1 + Amyα3 |
| C-Amyα3 | Amyα1 + Amyα4 |
| C-Amyα4 | Amyα1 + Amyα5 |
| C-Amyα5 | Amyα1 + Amyα6 |
| C-Amyα6 | Amyα1 + Amyα7 |
| C-Amyα7 | Amyα1 + Amyα8 |
| C-Amyα8 | Amyα1 + Amyα9 |
| C-Amyα9 | Amyα1 + Amyα10 |
| C-Amyα10 | Amyα1 + Amyα11 |
| C-Amyα11 | Amyα1 + Amyα12 |
| C-Amyα12 | Amyα1 + Amyα13 |
| C-Amyα13 | Amyα1 + Amyα14 |
| C-Amyα14 | Amyα1 + Amyα15 |
| C-Amyα15 | Amyα1 + Amyα16 |
| C-Amyα16 | Amyα1 + Amyα17 |
| C-Amyα17 | Amyα1 + Amyα18 |
| C-Amyα18 | Amyα1 + Amyα19 |
| C-Amyα19 | Amyα2 + Amyα3 |
| C-Amyα20 | Amyα2 + Amyα4 |
| C-Amyα21 | Amyα2 + Amyα5 |
| C-Amyα22 | Amyα2 + Amyα6 |
| C-Amyα23 | Amyα2 + Amyα7 |
| C-Amyα24 | Amyα2 + Amyα8 |
| C-Amyα25 | Amyα2 + Amyα9 |
| C-Amyα26 | Amyα2 + Amyα10 |
| C-Amyα27 | Amyα2 + Amyα11 |
| C-Amyα28 | Amyα2 + Amyα12 |
| C-Amyα29 | Amyα2 + Amyα13 |
| C-Amyα30 | Amyα2 + Amyα14 |
| C-Amyα31 | Amyα2 + Amyα15 |
| C-Amyα32 | Amyα2 + Amyα16 |
| C-Amyα33 | Amyα2 + Amyα17 |
| C-Amyα34 | Amyα2 + Amyα18 |
| C-Amyα35 | Amyα2 + Amyα19 |
| C-Amyα36 | Amyα3 + Amyα4 |
| C-Amyα37 | Amyα3 + Amyα5 |
| C-Amyα38 | Amyα3 + Amyα6 |
| C-Amyα39 | Amyα3 + Amyα7 |
| C-Amyα40 | Amyα3 + Amyα8 |
| C-Amyα41 | Amyα3 + Amyα9 |
| C-Amyα42 | Amyα3 + Amyα10 |
| C-Amyα43 | Amyα3 + Amyα11 |
| C-Amyα44 | Amyα3 + Amyα12 |
| C-Amyα45 | Amyα3 + Amyα13 |
| C-Amyα46 | Amyα3 + Amyα14 |
| C-Amyα47 | Amyα3 + Amyα15 |
| C-Amyα48 | Amyα3 + Amyα16 |
| C-Amyα49 | Amyα3 + Amyα17 |
| C-Amyα50 | Amyα3 + Amyα18 |
| C-Amyα51 | Amyα3 + Amyα19 |
| C-Amyα52 | Amyα4 + Amyα5 |
| C-Amyα53 | Amyα4 + Amyα6 |
| C-Amyα54 | Amyα4 + Amyα7 |
| C-Amyα55 | Amyα4 + Amyα8 |
| C-Amyα56 | Amyα4 + Amyα9 |
| C-Amyα57 | Amyα4 + Amyα10 |
| C-Amyα58 | Amyα4 + Amyα11 |
| C-Amyα59 | Amyα4 + Amyα12 |
| C-Amyα60 | Amyα4 + Amyα13 |
| C-Amyα61 | Amyα4 + Amyα14 |
| C-Amyα62 | Amyα4 + Amyα15 |
| C-Amyα63 | Amyα4 + Amyα16 |
| C-Amyα64 | Amyα4 + Amyα17 |
| C-Amyα65 | Amyα4 + Amyα18 |
| C-Amyα66 | Amyα4 + Amyα19 |
| C-Amyα67 | Amyα5 + Amyα6 |
| C-Amyα68 | Amyα5 + Amyα7 |
| C-Amyα69 | Amyα5 + Amyα8 |
| C-Amyα70 | Amyα5 + Amyα9 |
| C-Amyα71 | Amyα5 + Amyα10 |
| C-Amyα72 | Amyα5 + Amyα11 |
| C-Amyα73 | Amyα5 + Amyα12 |
| C-Amyα74 | Amyα5 + Amyα13 |
| C-Amyα75 | Amyα5 + Amyα14 |
| C-Amyα76 | Amyα5 + Amyα15 |
| C-Amyα77 | Amyα5 + Amyα16 |
| C-Amyα78 | Amyα5 + Amyα17 |
| C-Amyα79 | Amyα5 + Amyα18 |
| C-Amyα80 | Amyα5 + Amyα19 |
| C-Amyα81 | Amyα6 + Amyα7 |
| C-Amyα82 | Amyα6 + Amyα8 |
| C-Amyα83 | Amyα6 + Amyα9 |
| C-Amyα84 | Amyα6 + Amyα10 |
| C-Amyα85 | Amyα6 + Amyα11 |
| C-Amyα86 | Amyα6 + Amyα12 |
| C-Amyα87 | Amyα6 + Amyα13 |
| C-Amyα88 | Amyα6 + Amyα14 |
| C-Amyα89 | Amyα6 + Amyα15 |
| C-Amyα90 | Amyα6 + Amyα16 |
| C-Amyα91 | Amyα6 + Amyα17 |
| C-Amyα92 | Amyα6 + Amyα18 |
| C-Amyα93 | Amyα6 + Amyα19 |
| C-Amyα94 | Amyα7 + Amyα8 |
| C-Amyα95 | Amyα7 + Amyα9 |
| C-Amyα96 | Amyα7 + Amyα10 |
| C-Amyα97 | Amyα7 + Amyα11 |
| C-Amyα98 | Amyα7 + Amyα12 |
| C-Amyα99 | Amyα7 + Amyα13 |
| C-Amyα100 | Amyα7 + Amyα14 |
| C-Amyα101 | Amyα7 + Amyα15 |
| C-Amyα102 | Amyα7 + Amyα16 |
| C-Amyα103 | Amyα7 + Amyα17 |
| C-Amyα104 | Amyα7 + Amyα18 |
| C-Amyα105 | Amyα7 + Amyα19 |
| C-Amyα106 | Amyα8 + Amyα9 |
| C-Amyα107 | Amyα8 + Amyα10 |
| C-Amyα108 | Amyα8 + Amyα11 |
| C-Amyα109 | Amyα8 + Amyα12 |
| C-Amyα110 | Amyα8 + Amyα13 |
| C-Amyα111 | Amyα8 + Amyα14 |
| C-Amyα112 | Amyα8 + Amyα15 |
| C-Amyα113 | Amyα8 + Amyα16 |
| C-Amyα114 | Amyα8 + Amyα17 |
| C-Amyα115 | Amyα8 + Amyα18 |
| C-Amyα116 | Amyα8 + Amyα19 |
| C-Amyα117 | Amyα9 + Amyα10 |
| C-Amyα118 | Amyα9 + Amyα11 |
| C-Amyα119 | Amyα9 + Amyα12 |
| C-Amyα120 | Amyα9 + Amyα13 |
| C-Amyα121 | Amyα9 + Amyα14 |
| C-Amyα122 | Amyα9 + Amyα15 |
| C-Amyα123 | Amyα9 + Amyα16 |
| C-Amyα124 | Amyα9 + Amyα17 |
| C-Amyα125 | Amyα9 + Amyα18 |
| C-Amyα126 | Amyα9 + Amyα19 |
| C-Amyα127 | Amyα10 + Amyα11 |
| C-Amyα128 | Amyα10 + Amyα12 |
| C-Amyα129 | Amyα10 + Amyα13 |
| C-Amyα130 | Amyα10 + Amyα14 |
| C-Amyα131 | Amyα10 + Amyα15 |
| C-Amyα132 | Amyα10 + Amyα16 |
| C-Amyα133 | Amyα10 + Amyα17 |
| C-Amyα134 | Amyα10 + Amyα18 |
| C-Amyα135 | Amyα10 + Amyα19 |
| C-Amyα136 | Amyα11 + Amyα12 |
| C-Amyα137 | Amyα11 + Amyα13 |
| C-Amyα138 | Amyα11 + Amyα14 |
| C-Amyα139 | Amyα11 + Amyα15 |
| C-Amyα140 | Amyα11 + Amyα16 |
| C-Amyα141 | Amyα11 + Amyα17 |
| C-Amyα142 | Amyα11 + Amyα18 |
| C-Amyα143 | Amyα11 + Amyα19 |
| C-Amyα144 | Amyα12 + Amyα13 |
| C-Amyα145 | Amyα12 + Amyα14 |
| C-Amyα146 | Amyα12 + Amyα15 |
| C-Amyα147 | Amyα12 + Amyα16 |
| C-Amyα148 | Amyα12 + Amyα17 |
| C-Amyα149 | Amyα12 + Amyα18 |
| C-Amyα150 | Amyα12 + Amyα19 |
| C-Amyα151 | Amyα13 + Amyα14 |
| C-Amyα152 | Amyα13 + Amyα15 |
| C-Amyα153 | Amyα13 + Amyα16 |
| C-Amyα154 | Amyα13 + Amyα17 |
| C-Amyα155 | Amyα13 + Amyα18 |
| C-Amyα156 | Amyα13 + Amyα19 |
| C-Amyα157 | Amyα14 + Amyα15 |
| C-Amyα158 | Amyα14 + Amyα16 |
| C-Amyα159 | Amyα14 + Amyα17 |
| C-Amyα160 | Amyα14 + Amyα18 |
| C-Amyα161 | Amyα14 + Amyα19 |
| C-Amyα162 | Amyα15 + Amyα16 |
| C-Amyα163 | Amyα15 + Amyα17 |
| C-Amyα164 | Amyα15 + Amyα18 |
| C-Amyα165 | Amyα15 + Amyα19 |
| C-Amyα166 | Amyα16 + Amyα17 |
| C-Amyα167 | Amyα16 + Amyα18 |
| C-Amyα168 | Amyα16 + Amyα19 |
| C-Amyα169 | Amyα17 + Amyα18 |
| C-Amyα170 | Amyα17 + Amyα19 |
| C-Amyα171 | Amyα18 + Amyα19 |

### Lipases

“Lipase”, “lipolytic enzyme”, and “lipid esterase” all refer to an enzyme of the EC class 3.1.1 (“carboxylic ester hydrolase”). Lipase means enzymes having lipase activity or lipolytic activity (triacylglycerol lipase, EC 3.1.1.3), cutinase activity (EC 3.1.1.74; enzymes having cutinase activity may be called cutinase herein), sterol esterase activity (EC 3.1.1.13) and/or wax-ester hydrolase activity (EC 3.1.1.50). Lipases may be parent lipases or variants thereof. Parent lipases as well as variant lipases have to have lipase activity to be lipases according to the invention.

LEP or LDF, in one embodiment, comprise at least one lipase selected from triacylglycerol lipase (EC class 3.1.1.3). Preferably, triacylglycerol lipase is selected from lipases of Thermomyces lanuginosa. In one embodiment, LEP or LDF comprise at least one lipase selected from triacylglycerol lipase according to amino acids 1-269 of SEQ ID NO: 2 of U.S. Pat. No. 5,869,438 and variants thereof, preferably variants which are at least 90%, at least 91%, at least 92%, at least 93%, at least 94%, at least 95%, at least 96%, at least 97%, at least 98% or at least 99% identical when compared to the full length polypeptide sequence of amino acids 1-269 of SEQ ID NO: 2 of U.S. Pat. No. 5,869,438 (Lip1).

In one embodiment, LEP or LDF comprise at least one lipase selected from lipases having a polypeptide sequence which is at least 95%, at least 96%, at least 97%, at least 98%, or at least 99% identical to amino acids 1-269 of SEQ ID NO: 2 of U.S. Pat. No. 5,869,438 comprising at least the amino acid substitutions T231R and N233R (Lip1a). Said lipase variants may further comprise one or more of the following amino acid exchanges when compared to amino acids 1-269 of SEQ ID NO: 2 of U.S. Pat. No. 5,869,438: Q4V, V60S, A150G, L227G, P256K (Lip1b).

In one embodiment, LEP or LDF comprise at least one lipase at least 95% identical to the full-length polypeptide sequence of amino acids 1-269 of SEQ ID NO: 1 of WO 2015/010009 (Lip2), preferably comprising at least the amino acid substitutions N11K+A18K+G23K+K24A+V77I+D130A+V1541+V187T+T189Q (Lip2a) or N11K+A18K+G23K+K24A+L75R+V77I+D130A+V154|+V187T+T189Q (Lip2b).

### Combinations of Protease, Amylase and Lipase

In one embodiment, LEP or LDF comprise a combination of different types of hydrolases selected from protease, amylase and lipase, preferably at least one protease and at least one amylase, or at least one protease and at least one lipase or at least one protease, at least one amylase, and at least one lipase.

In one embodiment, LEP or LDF comprise at least one protease and at least one amylase, preferably at least one EPr9 and at least one amylase, more preferably at least one EPr9i and at least one Amyα.

In one embodiment, LEP or LDF comprise at least one protease and at least one lipase, preferably at least one EPr9 and at least one Lip1, more preferably at least one EPr9i and at least one Lip1a.

In one embodiment, LEP or LDF comprise at least one protease and at least one lipase, preferably at least one EPr9 and at least one Lip2, more preferably at least one EPr9i and at least one Lip2a.

In one embodiment, LEP or LDF comprise at least one lipase and at least one amylase, preferably at least one Lip1 and at least one amylase, more preferably at least one Lip1a and at least one Amyα.

In one embodiment, LEP or LDF comprise at least one lipase and at least one amylase, preferably at least one Lip2 and at least one amylase, more preferably at least one Lip2a and at least one Amyα.

Specifically, LEP or LDF may comprise one of the following combinations:

| identifier | combination |
|---|---|
| C-Hyd1 | EPr9a + Amyα1 |
| C-Hyd2 | EPr9a + Amyα2 |
| C-Hyd3 | EPr9a + Amyα3 |
| C-Hyd4 | EPr9a + Amyα4 |
| C-Hyd5 | EPr9a + Amyα5 |
| C-Hyd6 | EPr9a + Amyα6 |
| C-Hyd7 | EPr9a + Amyα7 |
| C-Hyd8 | EPr9a + Amyα8 |
| C-Hyd9 | EPr9a + Amyα9 |
| C-Hyd10 | EPr9a + Amyα10 |
| C-Hyd11 | EPr9a + Amyα11 |
| C-Hyd12 | EPr9a + Amyα12 |
| C-Hyd13 | EPr9a + Amyα13 |
| C-Hyd14 | EPr9a + Amyα14 |
| C-Hyd15 | EPr9a + Amyα15 |
| C-Hyd16 | EPr9a + Amyα16 |
| C-Hyd17 | EPr9a + Amyα17 |
| C-Hyd18 | EPr9a + Amyα18 |
| C-Hyd19 | EPr9a + Amyα19 |
| C-Hyd20 | EPr9b + Amyα1 |
| C-Hyd21 | EPr9b + Amyα2 |
| C-Hyd22 | EPr9b + Amyα3 |
| C-Hyd23 | EPr9b + Amyα4 |
| C-Hyd24 | EPr9b + Amyα5 |
| C-Hyd25 | EPr9b + Amyα6 |
| C-Hyd26 | EPr9b + Amyα7 |
| C-Hyd27 | EPr9b + Amyα8 |
| C-Hyd28 | EPr9b + Amyα9 |
| C-Hyd29 | EPr9b + Amyα10 |
| C-Hyd30 | EPr9b + Amyα11 |
| C-Hyd31 | EPr9b + Amyα12 |
| C-Hyd32 | EPr9b + Amyα13 |
| C-Hyd33 | EPr9b + Amyα14 |
| C-Hyd34 | EPr9b + Amyα15 |
| C-Hyd35 | EPr9b + Amyα16 |
| C-Hyd36 | EPr9b + Amyα17 |
| C-Hyd37 | EPr9b + Amyα18 |
| C-Hyd38 | EPr9b + Amyα19 |
| C-Hyd39 | EPr9c + Amyα1 |
| C-Hyd40 | EPr9c + Amyα2 |
| C-Hyd41 | EPr9c + Amyα3 |
| C-Hyd42 | EPr9c + Amyα4 |
| C-Hyd43 | EPr9c + Amyα5 |
| C-Hyd44 | EPr9c + Amyα6 |
| C-Hyd45 | EPr9c + Amyα7 |
| C-Hyd46 | EPr9c + Amyα8 |
| C-Hyd47 | EPr9c + Amyα9 |
| C-Hyd48 | EPr9c + Amyα10 |
| C-Hyd49 | EPr9c + Amyα11 |
| C-Hyd50 | EPr9c + Amyα12 |
| C-Hyd51 | EPr9c + Amyα13 |
| C-Hyd52 | EPr9c + Amyα14 |
| C-Hyd53 | EPr9c + Amyα15 |
| C-Hyd54 | EPr9c + Amyα16 |
| C-Hyd55 | EPr9c + Amyα17 |
| C-Hyd56 | EPr9c + Amyα18 |
| C-Hyd57 | EPr9c + Amyα19 |
| C-Hyd58 | EPr9d + Amyα1 |
| C-Hyd59 | EPr9d + Amyα2 |
| C-Hyd60 | EPr9d + Amyα3 |
| C-Hyd61 | EPr9d + Amyα4 |
| C-Hyd62 | EPr9d + Amyα5 |
| C-Hyd63 | EPr9d + Amyα6 |
| C-Hyd64 | EPr9d + Amyα7 |
| C-Hyd65 | EPr9d + Amyα8 |
| C-Hyd66 | EPr9d + Amyα9 |
| C-Hyd67 | EPr9d + Amyα10 |
| C-Hyd68 | EPr9d + Amyα11 |
| C-Hyd69 | EPr9d + Amyα12 |
| C-Hyd70 | EPr9d + Amyα13 |
| C-Hyd71 | EPr9d + Amyα14 |
| C-Hyd72 | EPr9d + Amyα15 |
| C-Hyd73 | EPr9d + Amyα16 |
| C-Hyd74 | EPr9d + Amyα17 |
| C-Hyd75 | EPr9d + Amyα18 |
| C-Hyd76 | EPr9d + Amyα19 |
| C-Hyd77 | EPr9e + Amyα1 |
| C-Hyd78 | EPr9e + Amyα2 |
| C-Hyd79 | EPr9e + Amyα3 |
| C-Hyd80 | EPr9e + Amyα4 |
| C-Hyd81 | EPr9e + Amyα5 |
| C-Hyd82 | EPr9e + Amyα6 |
| C-Hyd83 | EPr9e + Amyα7 |
| C-Hyd84 | EPr9e + Amyα8 |
| C-Hyd85 | EPr9e + Amyα9 |
| C-Hyd86 | EPr9e + Amyα10 |
| C-Hyd87 | EPr9e + Amyα11 |
| C-Hyd88 | EPr9e + Amyα12 |
| C-Hyd89 | EPr9e + Amyα13 |
| C-Hyd90 | EPr9e + Amyα14 |
| C-Hyd91 | EPr9e + Amyα15 |
| C-Hyd92 | EPr9e + Amyα16 |
| C-Hyd93 | EPr9e + Amyα17 |
| C-Hyd94 | EPr9e + Amyα18 |
| C-Hyd95 | EPr9e + Amyα19 |
| C-Hyd96 | EPr9f + Amyα1 |
| C-Hyd97 | EPr9f + Amyα2 |
| C-Hyd98 | EPr9f + Amyα3 |
| C-Hyd99 | EPr9f + Amyα4 |
| C-Hyd100 | EPr9f + Amyα5 |
| C-Hyd101 | EPr9f + Amyα6 |
| C-Hyd102 | EPr9f + Amyα7 |
| C-Hyd103 | EPr9f + Amyα8 |
| C-Hyd104 | EPr9f + Amyα9 |
| C-Hyd105 | EPr9f + Amyα10 |
| C-Hyd106 | EPr9f + Amyα11 |
| C-Hyd107 | EPr9f + Amyα12 |
| C-Hyd108 | EPr9f + Amyα13 |
| C-Hyd109 | EPr9f + Amyα14 |
| C-Hyd110 | EPr9f + Amyα15 |
| C-Hyd111 | EPr9f + Amyα16 |
| C-Hyd112 | EPr9f + Amyα17 |
| C-Hyd113 | EPr9f + Amyα18 |
| C-Hyd114 | EPr9f + Amyα19 |
| C-Hyd115 | EPr9g + Amyα1 |
| C-Hyd116 | EPr9g + Amyα2 |
| C-Hyd117 | EPr9g + Amyα3 |
| C-Hyd118 | EPr9g + Amyα4 |
| C-Hyd119 | EPr9g + Amyα5 |
| C-Hyd120 | EPr9g + Amyα6 |
| C-Hyd121 | EPr9g + Amyα7 |
| C-Hyd122 | EPr9g + Amyα8 |
| C-Hyd123 | EPr9g + Amyα9 |
| C-Hyd124 | EPr9g + Amyα10 |
| C-Hyd125 | EPr9g + Amyα11 |
| C-Hyd126 | EPr9g + Amyα12 |
| C-Hyd127 | EPr9g + Amyα13 |
| C-Hyd128 | EPr9g + Amyα14 |
| C-Hyd129 | EPr9g + Amyα15 |
| C-Hyd130 | EPr9g + Amyα16 |
| C-Hyd131 | EPr9g + Amyα17 |
| C-Hyd132 | EPr9g + Amyα18 |
| C-Hyd133 | EPr9g + Amyα19 |
| C-Hyd134 | EPr9h + Amyα1 |
| C-Hyd135 | EPr9h + Amyα2 |
| C-Hyd136 | EPr9h + Amyα3 |
| C-Hyd137 | EPr9h + Amyα4 |
| C-Hyd138 | EPr9h + Amyα5 |
| C-Hyd139 | EPr9h + Amyα6 |
| C-Hyd140 | EPr9h + Amyα7 |
| C-Hyd141 | EPr9h + Amyα8 |
| C-Hyd142 | EPr9h + Amyα9 |
| C-Hyd143 | EPr9h + Amyα10 |
| C-Hyd144 | EPr9h + Amyα11 |
| C-Hyd145 | EPr9h + Amyα12 |
| C-Hyd146 | EPr9h + Amyα13 |
| C-Hyd147 | EPr9h + Amyα14 |
| C-Hyd148 | EPr9h + Amyα15 |
| C-Hyd149 | EPr9h + Amyα16 |
| C-Hyd150 | EPr9h + Amyα17 |
| C-Hyd151 | EPr9h + Amyα18 |
| C-Hyd152 | EPr9h + Amyα19 |
| C-Hyd153 | EPr9i + Amyα1 |
| C-Hyd154 | EPr9i + Amyα2 |
| C-Hyd155 | EPr9i + Amyα3 |
| C-Hyd156 | EPr9i + Amyα4 |
| C-Hyd157 | EPr9i + Amyα5 |
| C-Hyd158 | EPr9i + Amyα6 |
| C-Hyd159 | EPr9i + Amyα7 |
| C-Hyd160 | EPr9i + Amyα8 |
| C-Hyd161 | EPr9i + Amyα9 |
| C-Hyd162 | EPr9i + Amyα10 |
| C-Hyd163 | EPr9i + Amyα11 |
| C-Hyd164 | EPr9i + Amyα12 |
| C-Hyd165 | EPr9i + Amyα13 |
| C-Hyd166 | EPr9i + Amyα14 |
| C-Hyd167 | EPr9i + Amyα15 |
| C-Hyd168 | EPr9i + Amyα16 |
| C-Hyd169 | EPr9i + Amyα17 |
| C-Hyd170 | EPr9i + Amyα18 |
| C-Hyd171 | EPr9i + Amyα19 |
| C-Hyd172 | EPr9iA + Amyα1 |
| C-Hyd173 | EPr9iA + Amyα2 |
| C-Hyd174 | EPr9iA + Amyα3 |
| C-Hyd175 | EPr9iA + Amyα4 |
| C-Hyd176 | EPr9iA + Amyα5 |
| C-Hyd177 | EPr9iA + Amyα6 |
| C-Hyd178 | EPr9iA + Amyα7 |
| C-Hyd179 | EPr9iA + Amyα8 |
| C-Hyd180 | EPr9iA + Amyα9 |
| C-Hyd181 | EPr9iA + Amyα10 |
| C-Hyd182 | EPr9iA + Amyα11 |
| C-Hyd183 | EPr9iA + Amyα12 |
| C-Hyd184 | EPr9iA + Amyα13 |
| C-Hyd185 | EPr9iA + Amyα14 |
| C-Hyd186 | EPr9iA + Amyα15 |
| C-Hyd187 | EPr9iA + Amyα16 |
| C-Hyd188 | EPr9iA + Amyα17 |
| C-Hyd189 | EPr9iA + Amyα18 |
| C-Hyd190 | EPr9iA + Amyα19 |
| C-Hyd191 | EPr9a + Lip1a |
| C-Hyd192 | EPr9b + Lip1a |
| C-Hyd193 | EPr9c + Lip1a |
| C-Hyd194 | EPr9d + Lip1a |
| C-Hyd195 | EPr9e + Lip1a |
| C-Hyd196 | EPr9f + Lip1a |
| C-Hyd197 | EPr9g + Lip1a |
| C-Hyd198 | EPr9h + Lip1a |
| C-Hyd199 | EPr9i + Lip1a |
| C-Hyd200 | EPr9iA + Lip1a |
| C-Hyd201 | EPr9a + Lip2a |
| C-Hyd202 | EPr9b + Lip2a |
| C-Hyd203 | EPr9c + Lip2a |
| C-Hyd204 | EPr9d + Lip2a |
| C-Hyd205 | EPr9e + Lip2a |
| C-Hyd206 | EPr9f + Lip2a |
| C-Hyd207 | EPr9g + Lip2a |
| C-Hyd208 | EPr9h + Lip2a |
| C-Hyd209 | EPr9i + Lip2a |
| C-Hyd210 | EPr9iA + Lip2a |
| C-Hyd211 | Lip1a + Amyα1 |
| C-Hyd212 | Lip1a + Amyα2 |
| C-Hyd213 | Lip1a + Amyα3 |
| C-Hyd214 | Lip1a + Amyα4 |
| C-Hyd215 | Lip1a + Amyα5 |
| C-Hyd216 | Lip1a + Amyα6 |
| C-Hyd217 | Lip1a + Amyα7 |
| C-Hyd218 | Lip1a + Amyα8 |
| C-Hyd219 | Lip1a + Amyα9 |
| C-Hyd220 | Lip1a + Amyα10 |
| C-Hyd221 | Lip1a + Amyα11 |
| C-Hyd222 | Lip1a + Amyα12 |
| C-Hyd223 | Lip1a + Amyα13 |
| C-Hyd224 | Lip1a + Amyα14 |
| C-Hyd225 | Lip1a + Amyα15 |
| C-Hyd226 | Lip1a + Amyα16 |
| C-Hyd227 | Lip1a + Amyα17 |
| C-Hyd228 | Lip1a + Amyα18 |
| C-Hyd229 | Lip1a + Amyα19 |
| C-Hyd230 | Lip2a + Amyα1 |
| C-Hyd231 | Lip2a + Amyα2 |
| C-Hyd232 | Lip2a + Amyα3 |
| C-Hyd233 | Lip2a + Amyα4 |
| C-Hyd234 | Lip2a + Amyα5 |
| C-Hyd235 | Lip2a + Amyα6 |
| C-Hyd236 | Lip2a + Amyα7 |
| C-Hyd237 | Lip2a + Amyα8 |
| C-Hyd238 | Lip2a + Amyα9 |
| C-Hyd239 | Lip2a + Amyα10 |
| C-Hyd240 | Lip2a + Amyα11 |
| C-Hyd241 | Lip2a + Amyα12 |
| C-Hyd242 | Lip2a + Amyα13 |
| C-Hyd243 | Lip2a + Amyα14 |
| C-Hyd244 | Lip2a + Amyα15 |
| C-Hyd245 | Lip2a + Amyα16 |
| C-Hyd246 | Lip2a + Amyα17 |
| C-Hyd247 | Lip2a + Amyα18 |
| C-Hyd248 | Lip2a + Amyα19 |

In one embodiment, EPr9iA in combinations C-Hyd172 to C-Hyd190, C-Hyd200 and CHyd210 has at least a substitution at position 101, preferably selected from R101E, R101D and R101S (according to BPN′ numbering).

In one embodiment, EPr9iA in combinations C-Hyd172 to C-Hyd190, C-Hyd200 and CHyd210 has one or more substitutions selected from 3T, 41, 63A/T/R, 156D/E, 194P, 199M, 205I and 217D/E/G, optionally together with a substitution at position 101 selected from R101E, R101D and R101S, wherein the numbering is according to the BPN′ numbering. In one embodiment, EPr9iA in combinations C-Hyd172 to C-Hyd190, C-Hyd200 and CHyd210 has one or more substitutions selected from S156D, L262E, Q137H, S3T, R45E/D/Q, P55N, T58W,Y,L, Q59D/M/N/T, G61D/R, S87E, G97S, A98D/E/R, S106A/W, N117E, H120V/D/K/N, S125M, P129D, E136Q, S144W, S161T, S163A/G, Y171L, A172S, N185Q, V199M, Y209W, M222Q, N238H, V244T, N261T/D and L262N/Q/D, and optionally a substitution at position 101 selected from R101E, R101D and R101S, and wherein the numbering is according to the BPN′ numbering.

### Component b./(B)—Alkanolamine Formate

Liquid formulations, e.g., LEP or LDF, of the invention comprise at least one compound according to formula (I), an alkanolamine formate (AFF):

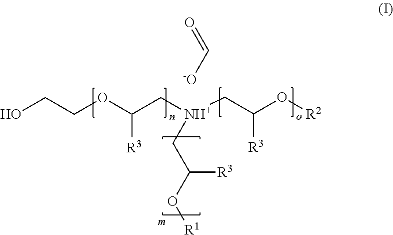

wherein R₁ and R₂ are selected from H and C₂H₄OH,
each of R₃ is independently selected from H, methyl and ethyl, preferably all R₃ are either H or methyl and m, n, o are each individually 0-2, preferably 0-1, more preferably 0.

In one embodiment, R₁ and R₂ in general formula (I) are H, which is called AAF1 herein.

Preferably, m, n, o are 0 (i. e. AAF1a).

In one embodiment, R₁, R₂ and R₃ in general formula (I) are H, m and o are 0, and n is 1 (i. e. AAF1b).

In one embodiment, R₁ and R₂ in general formula (I) are H, R₃ is methyl, m and o are 0 and n is 1 (i. e. AAF1c).

In one embodiment, R₁ in general formula (I) is H and R₂ is C₂H₄OH, which is called AAF2 herein. Preferably, m, n, o are each individually 0 (i. e. AAF2a).

In one embodiment, R₁ and R₃ in general formula (I) are H, R₂ is C₂H₄OH, m is 0, and n and o are 1 (i. e. AAF2b).

In one embodiment, R₁ in general formula (I) is H, R₂ is C₂H₄OH, R₃ is methyl, m is 0, and n and o are 1 (i. e. AAF2c).

In one embodiment, R₁ and R₂ in general formula (I) are C₂H₄OH, which is called AAF3 herein. Preferably, m, n, o are 0 (i. e. AAF3a also called triethanolamine formate).

In one embodiment, R₁ and R₂ in general formula (I) are C₂H₄OH, R₃ is H, and m, n, o are all 1 (i. e. AAF3b).

In one embodiment, R₁ and R₂ in general formula (I) are C₂H₄OH, R₃ is methyl, and m, n, o are all 1 (i. e. AAF3c).

### Component a./(A)+Component b./(B)

LEP or LDF according to the invention may comprise one of the following combinations:

In one embodiment, LEP or LDF of the invention comprise at least one protease and at least one AAF selected from AAF1, AAF2 and AAF3, preferably selected from AAF1a, AAF2a and AAF3a.

In one embodiment, LEP or LDF of the invention comprise at least one protease and at least one AAF selected from AAF1b, AAF2b and AAF3b.

In one embodiment, LEP or LDF of the invention comprise at least one protease and at least one AAF selected from AAF1c, AAF2c and AAF3c.

Specifically, LEP or LDF may comprise one of the following combinations:

| identifier | combination |
|---|---|
| LF1 | EPr9a + AAF1a |
| LF2 | EPr9a + AAF1b |
| LF3 | EPr9a + AAF1c |
| LF4 | EPr9a + AAF2a |
| LF5 | EPr9a + AAF2b |
| LF6 | EPr9a + AAF2c |
| LF7 | EPr9a + AAF3a |
| LF8 | EPr9a + AAF3b |
| LF9 | EPr9a + AAF3c |
| LF10 | EPr9b + AAF1a |
| LF11 | EPr9b + AAF1b |
| LF12 | EPr9b + AAF1c |
| LF13 | EPr9b + AAF2a |
| LF14 | EPr9b + AAF2b |
| LF15 | EPr9b + AAF2c |
| LF16 | EPr9b + AAF3a |
| LF17 | EPr9b + AAF3b |
| LF18 | EPr9b + AAF3c |
| LF19 | EPr9c + AAF1a |
| LF20 | EPr9c + AAF1b |
| LF21 | EPr9c + AAF1c |
| LF22 | EPr9c + AAF2a |
| LF23 | EPr9c + AAF2b |
| LF24 | EPr9c + AAF2c |
| LF25 | EPr9c + AAF3a |
| LF26 | EPr9c + AAF3b |
| LF27 | EPr9c + AAF3c |
| LF28 | EPr9d + AAF1a |
| LF29 | EPr9d + AAF1b |
| LF30 | EPr9d + AAF1c |
| LF31 | EPr9d + AAF2a |
| LF32 | EPr9d + AAF2b |
| LF33 | EPr9d + AAF2c |
| LF34 | EPr9d + AAF3a |
| LF35 | EPr9d + AAF3b |
| LF36 | EPr9d + AAF3c |
| LF37 | EPr9e + AAF1a |
| LF38 | EPr9e + AAF1b |
| LF39 | EPr9e + AAF1c |
| LF40 | EPr9e + AAF2a |
| LF41 | EPr9e + AAF2b |
| LF42 | EPr9e + AAF2c |
| LF43 | EPr9e + AAF3a |
| LF44 | EPr9e + AAF3b |
| LF45 | EPr9e + AAF3c |
| LF46 | EPr9f + AAF1a |
| LF47 | EPr9f + AAF1b |
| LF48 | EPr9f + AAF1c |
| LF49 | EPr9f + AAF2a |
| LF50 | EPr9f + AAF2b |
| LF51 | EPr9f + AAF2c |
| LF52 | EPr9f + AAF3a |
| LF53 | EPr9f + AAF3b |
| LF54 | EPr9f + AAF3c |
| LF55 | EPr9g + AAF1a |
| LF56 | EPr9g + AAF1b |
| LF57 | EPr9g + AAF1c |
| LF58 | EPr9g + AAF2a |
| LF59 | EPr9g + AAF2b |
| LF60 | EPr9g + AAF2c |
| LF61 | EPr9g + AAF3a |
| LF62 | EPr9g + AAF3b |
| LF63 | EPr9g + AAF3c |
| LF64 | EPr9h + AAF1a |
| LF65 | EPr9h + AAF1b |
| LF66 | EPr9h + AAF1c |
| LF67 | EPr9h + AAF2a |
| LF68 | EPr9h + AAF2b |
| LF69 | EPr9h + AAF2c |
| LF70 | EPr9h + AAF3a |
| LF71 | EPr9h + AAF3b |
| LF72 | EPr9h + AAF3c |
| LF73 | EPr9i + AAF1a |
| LF74 | EPr9i + AAF1b |
| LF77 | EPr9i + AAF1c |
| LF76 | EPr9i + AAF2a |
| LF77 | EPr9i + AAF2b |
| LF78 | EPr9i + AAF2c |
| LF79 | EPr9i + AAF3a |
| LF80 | EPr9i + AAF3b |
| LF81 | EPr9i + AAF3c |
| LF82 | EPr9iA + AAF1a |
| LF83 | EPr9iA + AAF1b |
| LF84 | EPr9iA + AAF1c |
| LF88 | EPr9iA + AAF2a |
| LF86 | EPr9iA + AAF2b |
| LF87 | EPr9iA + AAF2c |
| LF88 | EPr9iA + AAF3a |
| LF89 | EPr9iA + AAF3b |
| LF90 | EPr9iA + AAF3c |

In one embodiment, LEP or LDF of the invention comprise one of the combinations C-Pr1 to C-Pr44 and at least one AAF selected from AAF1a, AAF2a and AAF3a.

In one embodiment, LEP or LDF of the invention comprise one of the combinations C-Pr1 to C-Pr44 and at least one AAF selected from AAF1b, AAF2b and AAF3b.

In one embodiment, LEP or LDF of the invention comprise one of the combinations C-Pr1 to C-Pr44 and at least one AAF selected from AAF1c, AAF2c and AAF3c.

In one embodiment, LEP or LDF comprise
(A) at least one serine protease EPr9iA having at least the substitution R101E or R101D or R101S (according to BPN′ numbering), and
(B) AAF3a.

In one embodiment, LEP or LDF comprise
(A) at least one EPr9iA having one or more substitutions selected from 3T, 41, 63A/T/R, 156D/E, 194P, 199M, 205I and 217D/E/G, optionally together with a substitution R101E or R101D or R101S (wherein the numbering is according to the BPN′ numbering) and
(B) AAF3a.

In one embodiment, LEP or LDF comprise
(A) at least one EPr9iA having one or more substitutions selected from S156D, L262E, Q137H, S3T, R45E/D/Q, P55N, T58W/Y/L, Q59D,M,N,T, G61D/R, S87E, G97S, A98D/E/R, S106A/W, N117E, H120V/D/K/N, S125M, P129D, E136Q, S144W, S161T, S163A/G, Y171L, A172S, N185Q, V199M, Y209W, M222Q, N238H, V244T, N261T/D and L262N/Q/D, optionally together with a substitution R101E or R101D or R101S (wherein the numbering is according to the BPN′ numbering) and
(B) AAF3a.

In one embodiment, LEP or LDF comprise
(A) at least one EPr9iA having combinations of mutations selected from S3T+V4I+V205I, S3T+V4I+R101E+V205I, and S3T+V4I+V199M+V205|+L217D (according to BPN′ numbering), and
(B) AAF3a.

In one embodiment, LEP or LDF comprise
(A) at least one EPr9iA having mutations S3T+V4|+S9R+A15T+V68A+D99S+R101S+A103S+I104V+N218D (according to BPN′ numbering), and
(B) AAF3a.

In one embodiment, LEP or LDF of the invention comprise at least one amylase and at least one AAF selected from AAF1, AAF2 and AAF3, preferably selected from AAF1a, AAF2a and AAF3a.

In one embodiment, LEP or LDF of the invention comprise at least one amylase and at least one AAF selected from AAF1b, AAF2b and AAF3b.

In one embodiment, LEP or LDF of the invention comprise at least one amylase and at least one AAF selected from AAF1c, AAF2c and AAF3c.

Specifically, LEP or LDF may comprise one of the following combinations:

| id | combination |
|---|---|
| LF91 | Amyα1 + AAF1a |
| LF92 | Amyα1 + AAF1b |
| LF93 | Amyα1 + AAF1c |
| LF94 | Amyα1 + AAF2a |
| LF99 | Amyα1 + AAF2b |
| LF96 | Amyα1 + AAF2c |
| LF97 | Amyα1 + AAF3a |
| LF98 | Amyα1 + AAF3b |
| LF99 | Amyα1 + AAF3c |
| LF100 | Amyα2 + AAF1a |
| LF101 | Amyα2 + AAF1b |
| LF102 | Amyα2 + AAF1c |
| LF103 | Amyα2 + AAF2a |
| LF104 | Amyα2 + AAF2b |
| LF105 | Amyα2 + AAF2c |
| LF106 | Amyα2 + AAF3a |
| LF107 | Amyα2 + AAF3b |
| LF108 | Amyα2 + AAF3c |
| LF109 | Amyα3 + AAF1a |
| LF110 | Amyα3 + AAF1b |
| LF111 | Amyα3 + AAF1c |
| LF112 | Amyα3 + AAF2a |
| LF113 | Amyα3 + AAF2b |
| LF114 | Amyα3 + AAF2c |
| LF115 | Amyα3 + AAF3a |
| LF116 | Amyα3 + AAF3b |
| LF117 | Amyα3 + AAF3c |
| LF118 | Amyα4 + AAF1a |
| LF119 | Amyα4 + AAF1b |
| LF120 | Amyα4 + AAF1c |
| LF121 | Amyα4 + AAF2a |
| LF122 | Amyα4 + AAF2b |
| LF123 | Amyα4 + AAF2c |
| LF124 | Amyα4 + AAF3a |
| LF125 | Amyα4 + AAF3b |
| LF126 | Amyα4 + AAF3c |
| LF127 | Amyα5 + AAF1a |
| LF128 | Amyα5 + AAF1b |
| LF129 | Amyα5 + AAF1c |
| LF130 | Amyα5 + AAF2a |
| LF131 | Amyα5 + AAF2b |
| LF132 | Amyα5 + AAF2c |
| LF133 | Amyα5 + AAF3a |
| LF134 | Amyα5 + AAF3b |
| LF135 | Amyα5 + AAF3c |
| LF136 | Amyα6 + AAF1a |
| LF137 | Amyα6 + AAF1b |
| LF138 | Amyα6 + AAF1c |
| LF139 | Amyα6 + AAF2a |
| LF140 | Amyα6 + AAF2b |
| LF141 | Amyα6 + AAF2c |
| LF142 | Amyα6 + AAF3a |
| LF143 | Amyα6 + AAF3b |
| LF144 | Amyα6 + AAF3c |
| LF145 | Amyα7 + AAF1a |
| LF146 | Amyα7 + AAF1b |
| LF147 | Amyα7 + AAF1c |
| LF148 | Amyα7 + AAF2a |
| LF149 | Amyα7 + AAF2b |
| LF150 | Amyα7 + AAF2c |
| LF151 | Amyα7 + AAF3a |
| LF152 | Amyα7 + AAF3b |
| LF153 | Amyα7 + AAF3c |
| LF154 | Amyα8 + AAF1a |
| LF155 | Amyα8 + AAF1b |
| LF156 | Amyα8 + AAF1c |
| LF157 | Amyα8 + AAF2a |
| LF158 | Amyα8 + AAF2b |
| LF159 | Amyα8 + AAF2c |
| LF160 | Amyα8 + AAF3a |
| LF161 | Amyα8 + AAF3b |
| LF162 | Amyα8 + AAF3c |
| LF163 | Amyα9 + AAF1a |
| LF164 | Amyα9 + AAF1b |
| LF165 | Amyα9 + AAF1c |
| LF166 | Amyα9 + AAF2a |
| LF167 | Amyα9 + AAF2b |
| LF168 | Amyα9 + AAF2c |
| LF169 | Amyα9 + AAF3a |
| LF170 | Amyα9 + AAF3b |
| LF171 | Amyα9 + AAF3c |
| LF172 | Amyα10 + AAF1a |
| LF173 | Amyα10 + AAF1b |
| LF174 | Amyα10 + AAF1c |
| LF175 | Amyα10 + AAF2a |
| LF176 | Amyα10 + AAF2b |
| LF177 | Amyα10 + AAF2c |
| LF178 | Amyα10 + AAF3a |
| LF179 | Amyα10 + AAF3b |
| LF180 | Amyα10 + AAF3c |
| LF181 | Amyα11 + AAF1a |
| LF182 | Amyα11 + AAF1b |
| LF183 | Amyα11 + AAF1c |
| LF184 | Amyα11 + AAF2a |
| LF185 | Amyα11 + AAF2b |
| LF186 | Amyα11 + AAF2c |
| LF187 | Amyα11 + AAF3a |
| LF188 | Amyα11 + AAF3b |
| LF189 | Amyα11 + AAF3c |
| LF190 | Amyα12 + AAF1a |
| LF191 | Amyα12 + AAF1b |
| LF192 | Amyα12 + AAF1c |
| LF193 | Amyα12 + AAF2a |
| LF194 | Amyα12 + AAF2b |
| LF195 | Amyα12 + AAF2c |
| LF196 | Amyα12 + AAF3a |
| LF197 | Amyα12 + AAF3b |
| LF198 | Amyα12 + AAF3c |
| LF199 | Amyα13 + AAF1a |
| LF200 | Amyα13 + AAF1b |
| LF201 | Amyα13 + AAF1c |
| LF202 | Amyα13 + AAF2a |
| LF203 | Amyα13 + AAF2b |
| LF204 | Amyα13 + AAF2c |
| LF205 | Amyα13 + AAF3a |
| LF206 | Amyα13 + AAF3b |
| LF207 | Amyα13 + AAF3c |
| LF208 | Amyα14 + AAF1a |
| LF209 | Amyα14 + AAF1b |
| LF210 | Amyα14 + AAF1c |
| LF211 | Amyα14 + AAF2a |
| LF212 | Amyα14 + AAF2b |
| LF213 | Amyα14 + AAF2c |
| LF214 | Amyα14 + AAF3a |
| LF215 | Amyα14 + AAF3b |
| LF216 | Amyα14 + AAF3c |
| LF217 | Amyα15 + AAF1a |
| LF218 | Amyα15 + AAF1b |
| LF219 | Amyα15 + AAF1c |
| LF220 | Amyα15 + AAF2a |
| LF221 | Amyα15 + AAF2b |
| LF222 | Amyα15 + AAF2c |
| LF223 | Amyα15 + AAF3a |
| LF224 | Amyα15 + AAF3b |
| LF225 | Amyα15 + AAF3c |
| LF226 | Amyα16 + AAF1a |
| LF227 | Amyα16 + AAF1b |
| LF228 | Amyα16 + AAF1c |
| LF229 | Amyα16 + AAF2a |
| LF230 | Amyα16 + AAF2b |
| LF231 | Amyα16 + AAF2c |
| LF232 | Amyα16 + AAF3a |
| LF233 | Amyα16 + AAF3b |
| LF234 | Amyα16 + AAF3c |
| LF235 | Amyα17 + AAF1a |
| LF236 | Amyα17 + AAF1b |
| LF237 | Amyα17 + AAF1c |
| LF238 | Amyα17 + AAF2a |
| LF239 | Amyα17 + AAF2b |
| LF240 | Amyα17 + AAF2c |
| LF241 | Amyα17 + AAF3a |
| LF242 | Amyα17 + AAF3b |
| LF243 | Amyα17 + AAF3c |
| LF244 | Amyα18 + AAF1a |
| LF245 | Amyα18 + AAF1b |
| LF246 | Amyα18 + AAF1c |
| LF247 | Amyα18 + AAF2a |
| LF248 | Amyα18 + AAF2b |
| LF249 | Amyα18 + AAF2c |
| LF250 | Amyα18 + AAF3a |
| LF251 | Amyα18 + AAF3b |
| LF252 | Amyα18 + AAF3c |
| LF253 | Amyα19 + AAF1a |
| LF254 | Amyα19 + AAF1b |
| LF255 | Amyα19 + AAF1c |
| LF256 | Amyα19 + AAF2a |
| LF257 | Amyα19 + AAF2b |
| LF258 | Amyα19 + AAF2c |
| LF259 | Amyα19 + AAF3a |
| LF260 | Amyα19 + AAF3b |
| LF261 | Amyα19 + AAF3c |

In one embodiment, LEP or LDF of the invention comprise one of the combinations C-Amyα1 to C-Amyα171 and at least one AAF selected from AAF1a, AAF2a, AAF3a, AAF1b, AAF2b, AAF3b, AAF1c, AAF2c and AAF3c.

In one embodiment, LEP or LDF of the invention comprise at least one lipase and at least one AAF selected from AAF1, AAF2 and AAF3, preferably selected from AAF1a, AAF2a and AAF3a.

In one embodiment, LEP or LDF of the invention comprise at least one lipase and at least one AAF selected from AAF1b, AAF2b and AAF3b.

In one embodiment, LEP or LDF of the invention comprise at least one lipase and at least one AAF selected from AAF1c, AAF2c and AAF3c.

Specifically. LEP or LDF may comprise one of the following combinations:

| identifier | combination |
|---|---|
| LF262 | Lip1a + AAF1a |
| LF263 | Lip1a + AAF1b |
| LF264 | Lip1a + AAF1c |
| LF265 | Lip1a + AAF2a |
| LF266 | Lip1a + AAF2b |
| LF267 | Lip1a + AAF2c |
| LF268 | Lip1a + AAF3a |
| LF269 | Lip1a + AAF3b |
| LF270 | Lip1a + AAF3c |
| LF271 | Lip1b + AAF1a |
| LF272 | Lip1b + AAF1b |
| LF273 | Lip1b + AAF1c |
| LF274 | Lip1b + AAF2a |
| LF275 | Lip1b + AAF2b |
| LF276 | Lip1b + AAF2c |
| LF277 | Lip1b + AAF3a |
| LF278 | Lip1b + AAF3b |
| LF279 | Lip1b + AAF3c |

In one embodiment, LEP or LDF comprise a combination of different types of hydrolases, preferably according to one of the combinations C-Hyd1 to C-Hyd248 and at least one AAF selected from AAF1, AAF2 and AAF3, preferably selected from AAF1a, AAF2a, AAF3a, AAF1b, AAF2b, AAF3b AAF1c, AAF2c, and AAF3c.

Preferably. LEP or LDF comprise one of the following combinations:

| id | actual combinations |
|---|---|
| LF280 | AAF3a + C-Hyd1 |
| LF281 | AAF3a + C-Hyd2 |
| LF282 | AAF3a + C-Hyd3 |
| LF283 | AAF3a + C-Hyd4 |
| LF284 | AAF3a + C-Hyd5 |
| LF285 | AAF3a + C-Hyd6 |
| LF286 | AAF3a + C-Hyd7 |
| LF287 | AAF3a + C-Hyd8 |
| LF288 | AAF3a + C-Hyd9 |
| LF289 | AAF3a + C-Hyd10 |
| LF290 | AAF3a + C-Hyd11 |
| LF291 | AAF3a + C-Hyd12 |
| LF292 | AAF3a + C-Hyd13 |
| LF293 | AAF3a + C-Hyd14 |
| LF294 | AAF3a + C-Hyd15 |
| LF295 | AAF3a + C-Hyd16 |
| LF296 | AAF3a + C-Hyd17 |
| LF297 | AAF3a + C-Hyd18 |
| LF298 | AAF3a + C-Hyd19 |
| LF299 | AAF3a + C-Hyd20 |
| LF300 | AAF3a + C-Hyd21 |
| LF301 | AAF3a + C-Hyd22 |
| LF302 | AAF3a + C-Hyd23 |
| LF303 | AAF3a + C-Hyd24 |
| LF304 | AAF3a + C-Hyd25 |
| LF305 | AAF3a + C-Hyd26 |
| LF306 | AAF3a + C-Hyd27 |
| LF307 | AAF3a + C-Hyd28 |
| LF308 | AAF3a + C-Hyd29 |
| LF309 | AAF3a + C-Hyd30 |
| LF310 | AAF3a + C-Hyd31 |
| LF311 | AAF3a + C-Hyd32 |
| LF312 | AAF3a + C-Hyd33 |
| LF313 | AAF3a + C-Hyd34 |
| LF314 | AAF3a + C-Hyd35 |
| LF315 | AAF3a + C-Hyd36 |
| LF316 | AAF3a + C-Hyd37 |
| LF317 | AAF3a + C-Hyd38 |
| LF318 | AAF3a + C-Hyd39 |
| LF319 | AAF3a + C-Hyd40 |
| LF320 | AAF3a + C-Hyd41 |
| LF321 | AAF3a + C-Hyd42 |
| LF322 | AAF3a + C-Hyd43 |
| LF323 | AAF3a + C-Hyd44 |
| LF324 | AAF3a + C-Hyd45 |
| LF325 | AAF3a + C-Hyd46 |
| LF326 | AAF3a + C-Hyd47 |
| LF327 | AAF3a + C-Hyd48 |
| LF328 | AAF3a + C-Hyd49 |
| LF329 | AAF3a + C-Hyd50 |
| LF330 | AAF3a + C-Hyd51 |
| LF331 | AAF3a + C-Hyd52 |
| LF332 | AAF3a + C-Hyd53 |
| LF333 | AAF3a + C-Hyd54 |
| LF334 | AAF3a + C-Hyd55 |
| LF335 | AAF3a + C-Hyd56 |
| LF336 | AAF3a + C-Hyd57 |
| LF337 | AAF3a + C-Hyd58 |
| LF338 | AAF3a + C-Hyd59 |
| LF339 | AAF3a + C-Hyd60 |
| LF340 | AAF3a + C-Hyd61 |
| LF341 | AAF3a + C-Hyd62 |
| LF342 | AAF3a + C-Hyd63 |
| LF343 | AAF3a + C-Hyd64 |
| LF344 | AAF3a + C-Hyd65 |
| LF345 | AAF3a + C-Hyd66 |
| LF346 | AAF3a + C-Hyd67 |
| LF347 | AAF3a + C-Hyd68 |
| LF348 | AAF3a + C-Hyd69 |
| LF349 | AAF3a + C-Hyd70 |
| LF350 | AAF3a + C-Hyd71 |
| LF351 | AAF3a + C-Hyd72 |
| LF352 | AAF3a + C-Hyd73 |
| LF353 | AAF3a + C-Hyd74 |
| LF354 | AAF3a + C-Hyd75 |
| LF355 | AAF3a + C-Hyd76 |
| LF356 | AAF3a + C-Hyd77 |
| LF357 | AAF3a + C-Hyd78 |
| LF358 | AAF3a + C-Hyd79 |
| LF359 | AAF3a + C-Hyd80 |
| LF360 | AAF3a + C-Hyd81 |
| LF361 | AAF3a + C-Hyd82 |
| LF362 | AAF3a + C-Hyd83 |
| LF363 | AAF3a + C-Hyd84 |
| LF364 | AAF3a + C-Hyd85 |
| LF365 | AAF3a + C-Hyd86 |
| LF366 | AAF3a + C-Hyd87 |
| LF367 | AAF3a + C-Hyd88 |
| LF368 | AAF3a + C-Hyd89 |
| LF369 | AAF3a + C-Hyd90 |
| LF370 | AAF3a + C-Hyd91 |
| LF371 | AAF3a + C-Hyd92 |
| LF372 | AAF3a + C-Hyd93 |
| LF373 | AAF3a + C-Hyd94 |
| LF374 | AAF3a + C-Hyd95 |
| LF375 | AAF3a + C-Hyd96 |
| LF376 | AAF3a + C-Hyd97 |
| LF377 | AAF3a + C-Hyd98 |
| LF378 | AAF3a + C-Hyd99 |
| LF379 | AAF3a + C-Hyd100 |
| LF380 | AAF3a + C-Hyd101 |
| LF381 | AAF3a + C-Hyd102 |
| LF382 | AAF3a + C-Hyd103 |
| LF383 | AAF3a + C-Hyd104 |
| LF384 | AAF3a + C-Hyd105 |
| LF385 | AAF3a + C-Hyd106 |
| LF386 | AAF3a + C-Hyd107 |
| LF387 | AAF3a + C-Hyd108 |
| LF388 | AAF3a + C-Hyd109 |
| LF389 | AAF3a + C-Hyd110 |
| LF390 | AAF3a + C-Hyd111 |
| LF391 | AAF3a + C-Hyd112 |
| LF392 | AAF3a + C-Hyd113 |
| LF393 | AAF3a + C-Hyd114 |
| LF394 | AAF3a + C-Hyd115 |
| LF395 | AAF3a + C-Hyd116 |
| LF396 | AAF3a + C-Hyd117 |
| LF397 | AAF3a + C-Hyd118 |
| LF398 | AAF3a + C-Hyd119 |
| LF399 | AAF3a + C-Hyd120 |
| LF400 | AAF3a + C-Hyd121 |
| LF401 | AAF3a + C-Hyd122 |
| LF402 | AAF3a + C-Hyd123 |
| LF403 | AAF3a + C-Hyd124 |
| LF404 | AAF3a + C-Hyd125 |
| LF405 | AAF3a + C-Hyd126 |
| LF406 | AAF3a + C-Hyd127 |
| LF407 | AAF3a + C-Hyd128 |
| LF408 | AAF3a + C-Hyd129 |
| LF409 | AAF3a + C-Hyd130 |
| LF410 | AAF3a + C-Hyd131 |
| LF411 | AAF3a + C-Hyd132 |
| LF412 | AAF3a + C-Hyd133 |
| LF413 | AAF3a + C-Hyd134 |
| LF414 | AAF3a + C-Hyd135 |
| LF415 | AAF3a + C-Hyd136 |
| LF416 | AAF3a + C-Hyd137 |
| LF417 | AAF3a + C-Hyd138 |
| LF418 | AAF3a + C-Hyd139 |
| LF419 | AAF3a + C-Hyd140 |
| LF420 | AAF3a + C-Hyd141 |
| LF421 | AAF3a + C-Hyd142 |
| LF422 | AAF3a + C-Hyd143 |
| LF423 | AAF3a + C-Hyd144 |
| LF424 | AAF3a + C-Hyd145 |
| LF425 | AAF3a + C-Hyd146 |
| LF426 | AAF3a + C-Hyd147 |
| LF427 | AAF3a + C-Hyd148 |
| LF428 | AAF3a + C-Hyd149 |
| LF429 | AAF3a + C-Hyd150 |
| LF430 | AAF3a + C-Hyd151 |
| LF431 | AAF3a + C-Hyd152 |
| LF432 | AAF3a + C-Hyd153 |
| LF433 | AAF3a + C-Hyd154 |
| LF434 | AAF3a + C-Hyd155 |
| LF435 | AAF3a + C-Hyd156 |
| LF436 | AAF3a + C-Hyd157 |
| LF437 | AAF3a + C-Hyd158 |
| LF438 | AAF3a + C-Hyd159 |
| LF439 | AAF3a + C-Hyd160 |
| LF440 | AAF3a + C-Hyd161 |
| LF441 | AAF3a + C-Hyd162 |
| LF442 | AAF3a + C-Hyd163 |
| LF443 | AAF3a + C-Hyd164 |
| LF444 | AAF3a + C-Hyd165 |
| LF445 | AAF3a + C-Hyd166 |
| LF446 | AAF3a + C-Hyd167 |
| LF447 | AAF3a + C-Hyd168 |
| LF448 | AAF3a + C-Hyd169 |
| LF449 | AAF3a + C-Hyd170 |
| LF450 | AAF3a + C-Hyd171 |
| LF451 | AAF3a + C-Hyd172 |
| LF452 | AAF3a + C-Hyd173 |
| LF453 | AAF3a + C-Hyd174 |
| LF454 | AAF3a + C-Hyd175 |
| LF455 | AAF3a + C-Hyd176 |
| LF456 | AAF3a + C-Hyd177 |
| LF457 | AAF3a + C-Hyd178 |
| LF458 | AAF3a + C-Hyd179 |
| LF459 | AAF3a + C-Hyd180 |
| LF460 | AAF3a + C-Hyd181 |
| LF461 | AAF3a + C-Hyd182 |
| LF462 | AAF3a + C-Hyd183 |
| LF463 | AAF3a + C-Hyd184 |
| LF464 | AAF3a + C-Hyd185 |
| LF465 | AAF3a + C-Hyd186 |
| LF466 | AAF3a + C-Hyd187 |
| LF467 | AAF3a + C-Hyd188 |
| LF468 | AAF3a + C-Hyd189 |
| LF469 | AAF3a + C-Hyd190 |
| LF470 | AAF3a + C-Hyd191 |
| LF471 | AAF3a + C-Hyd192 |
| LF472 | AAF3a + C-Hyd193 |
| LF473 | AAF3a + C-Hyd194 |
| LF474 | AAF3a + C-Hyd195 |
| LF475 | AAF3a + C-Hyd196 |
| LF476 | AAF3a + C-Hyd197 |
| LF477 | AAF3a + C-Hyd198 |
| LF478 | AAF3a + C-Hyd199 |
| LF479 | AAF3a + C-Hyd200 |
| LF480 | AAF3a + C-Hyd201 |
| LF481 | AAF3a + C-Hyd202 |
| LF482 | AAF3a + C-Hyd203 |
| LF483 | AAF3a + C-Hyd204 |
| LF484 | AAF3a + C-Hyd205 |
| LF485 | AAF3a + C-Hyd206 |
| LF486 | AAF3a + C-Hyd207 |
| LF487 | AAF3a + C-Hyd208 |
| LF488 | AAF3a + C-Hyd209 |
| LF489 | AAF3a + C-Hyd210 |
| LF490 | AAF3a + C-Hyd211 |
| LF491 | AAF3a + C-Hyd212 |
| LF492 | AAF3a + C-Hyd213 |
| LF493 | AAF3a + C-Hyd214 |
| LF494 | AAF3a + C-Hyd215 |
| LF495 | AAF3a + C-Hyd216 |
| LF496 | AAF3a + C-Hyd217 |
| LF497 | AAF3a + C-Hyd218 |
| LF498 | AAF3a + C-Hyd219 |
| LF499 | AAF3a + C-Hyd220 |
| LF500 | AAF3a + C-Hyd221 |
| LF501 | AAF3a + C-Hyd222 |
| LF502 | AAF3a + C-Hyd223 |
| LF503 | AAF3a + C-Hyd224 |
| LF504 | AAF3a + C-Hyd225 |
| LF505 | AAF3a + C-Hyd226 |
| LF506 | AAF3a + C-Hyd227 |
| LF507 | AAF3a + C-Hyd228 |
| LF508 | AAF3a + C-Hyd229 |
| LF509 | AAF3a + C-Hyd230 |
| LF510 | AAF3a + C-Hyd231 |
| LF511 | AAF3a + C-Hyd232 |
| LF512 | AAF3a + C-Hyd233 |
| LF513 | AAF3a + C-Hyd234 |
| LF514 | AAF3a + C-Hyd235 |
| LF515 | AAF3a + C-Hyd236 |
| LF516 | AAF3a + C-Hyd237 |
| LF517 | AAF3a + C-Hyd238 |
| LF518 | AAF3a + C-Hyd239 |
| LF519 | AAF3a + C-Hyd240 |
| LF520 | AAF3a + C-Hyd241 |
| LF521 | AAF3a + C-Hyd242 |
| LF522 | AAF3a + C-Hyd243 |
| LF523 | AAF3a + C-Hyd244 |
| LF524 | AAF3a + C-Hyd245 |
| LF525 | AAF3a + C-Hyd246 |
| LF526 | AAF3a + C-Hyd247 |
| LF527 | AAF3a + C-Hyd248 |

In one embodiment, combinations LF280 to LF469 as disclosed above, further comprise Lip1a.

In one embodiment, combinations LF1 to LF527 as disclosed above comprise at least one further hydrolase different from proteases, amylases and lipases. Said hydrolase different from proteases, amylases and lipases is preferably selected from cellulases and mannanases.

LF1 to LF527, in one embodiment, comprise at least one cellulase, preferably at least one beta-1,4-glucanase (EC 3.2.1.4) which is also called endoglucanase herein.

In one embodiment, LF1 to LF527 comprise at least one Humicola insolens DSM 1800 endoglucanase at least 80% identical to the amino acid sequence disclosed in FIG. 14A-E of WO 91/17244, preferably to the sequence according to amino acids 20-434. Preferably said endoglucanase having one or more substitutions at positions selected from 182, 223, and 231, most preferably selected from P182S, A223V, and A231V. In one embodiment, LF1 to LF498 comprise at least one endoglucanase at least 80% identical to a polypeptide according to SEQ ID NO: 2 of WO 95/02675.

In one embodiment, LF1 to LF527 comprise at least one Bacillus sp. endoglucanase which is at least 80% identical to the amino acid sequence of position 1 to position 773 of SEQ ID NO: 2 of WO 2004/053039.

In one embodiment, LF1 to LF527 comprise at least one Thielavia terrestris endoglucanase which is at least 80% identical to the amino acid sequence of position 1 to position 299 of SEQ ID NO:4 of WO 2004/053039.

In one embodiment, LF1 to LF527 comprise at least one mannanase, preferably at least one beta-mannanase (EC 3.2.1.78).

In one embodiment, LF1 to LF527 comprise at least one beta-mannanase selected from GH5 family mannanase. In one embodiment, LF1 to LF527 comprise at least one beta-mannanase at least 90% identical to SEQ ID NO: 12 of WO 2018/184767. In one embodiment, LF1 to LF527 comprise at least one beta-mannanase at least 90% identical to SEQ ID NO: 16 of WO 2018/184767. In one embodiment, LF1 to LF527 comprise at least one beta-mannanase at least 90% identical to SEQ ID NO:20 of WO 2018/184767. Preferably, LF1 to LF527 comprise at least one mannanase 95% identical to a polypeptide sequence of SEQ ID NO: 20 of WO 2018/184767 having at least one substitution selected from A101V, E405G, and Y459F.

In one embodiment, LF1 to LF527 comprise at least one beta-mannanase originating from Trichoderma organisms, such as those disclosed in WO 93/24622. Preferably, at least one beta-mannanase is 80% identical to SEQ ID NO: 1 of WO 2008/009673. More preferably, the beta-mannanase according to SEQ ID NO: 1 of WO 2008/009673 comprises at least one substitution selected from S3R, S66P, N113Y, V181H, L207F, A215T and F274L.

In one embodiment, LF1 to LF527 comprise at least one beta-mannanase having a polypeptide sequence, which is at least 85% identical to SEQ ID NO: 16 as disclosed in WO 2018/185367.

In one embodiment, LF1 to LF527 comprise at least one beta-mannanase having a polypeptide sequence, which is at least 85% identical to SEQ ID NO: 12 as disclosed in WO 2018/185367.

In one embodiment, LF1 to LF527 comprise at least one beta-mannanase having a polypeptide sequence, which is at least 85% identical to SEQ ID NO: 20 as disclosed in WO 2018/185367.

In one embodiment, LF1 to LF527 comprise at least one beta-mannanase having a polypeptide sequence, which is at least 85% identical to SEQ ID NO: 388 as disclosed in WO 2005/003319.

In one embodiment, LEP according to the invention are essentially devoid surface active anionic compounds and complexing anionic compounds. “Essentially devoid of surface active anionic compounds and complexing anionic compounds” means that no such compound is added on purpose, meaning that preferably not more than 0.5%, more preferably not more than 0.01%, most preferably 0% surface active anionic compounds and complexing anionic compounds are contained. Surface active anionic compounds preferably means anionic surfactants.

Complexing anionic compounds preferably means citrates and aminocarboxylates.

In one embodiment, LEP or LDF according to the invention, preferably those comprising a protease such as LF1 to LF90 (preferably LF82 to LF90) and LF280 to LF489 (preferably LF451 to LF469, LF479 and LF489), comprise at least one enzyme stabilizer selected from boron-containing stabilizers and peptide stabilizers.

In one embodiment, LF82 to LF90 comprise 4-formyl phenyl boronic acid (4-FPBA). Preferably, at least one serine protease EPr9iA in LF82 to LF90 has at least the substitution R101E or R101D or R101S, preferably R101E (according to BPN′ numbering).

In one embodiment, LF451 to LF469, LF479 and LF489 comprise 4-formyl phenyl boronic acid (4-FPBA). Preferably, at least one serine protease EPr9iA in LF451 to LF469, LF479 and LF489 has at least the substitution R101E or R101D or R101S (according to BPN′ numbering).

In one embodiment, LF82 to LF90 comprise Z-VAL, preferably Z-VAL-H. Preferably, at least one serine protease EPr9iA in LF82 to LF90 has at least the substitution R101E or R101D or R101S (according to BPN′ numbering).

In one embodiment, LF451 to LF469, LF479 and LF489 comprise Z-VAL, preferably Z-VAL-H. Preferably, at least one serine protease EPr9iA in LF451 to LF469, LF479 and LF489 has at least the substitution R101E or R101D or R101S, preferably R101E (according to BPN′ numbering).

In a preferred embodiment, the LEP comprise a reduced amount of enzyme stabilizers selected from boron-containing stabilizers and/or peptide stabilizers. In a preferred embodiment, LEP are essentially devoid of enzyme stabilizers selected from boron-containing stabilizers and peptide stabilizers. “Essentially devoid of enzyme stabilizers” means that no such compound is added to the LEP on purpose, meaning that preferably not more than 0.5%, more preferably not more than 0.01%, most preferably 0% enzyme stabilizers are contained.

In a preferred embodiment, LEP comprises water in amounts not exceeding with increasing preference 50%, 40%, 30%, 20%, or 15% by weight.

In one embodiment, LEP or LDF of the invention comprise at least one salt of a monovalent cation and a monovalent anion of 1-6 carbons, preferably C1-3 carbons. Preferably, the monovalent cation is selected from Na⁺ (SALT1), K⁺ (SALT2) and NH₄⁺ (SALT3). Preferably, the monovalent anion is selected from formate (SALT #a), acetate (SALT #b), propionate (SALT #c) and lactate (SALT #d).

LEP or LDF, in one embodiment, comprise at least one salt selected from NaCl (SALT4), KCl (SALT5), CaCl₂) (SALT6) and Na₂SO₄ (SALT7).

In one embodiment, LF1 to LF527 comprise sodium formate (SALT1a) or SALT6. Preferably, at least one serine protease EPr9iA in LF82 to LF90 has at least the substitution R101E or R101D or R101S (according to BPN′ numbering). Preferably, at least one serine protease EPr9iA in LF451 to LF469, LF479 and LF489 has at least the substitution R101E or R101D or R101S (according to BPN′ numbering).

In a preferred embodiment, LDF comprises ≤3% by weight, preferably ≤2% by weight, more preferably ≤1% by weight sodium formate.

In one embodiment, LEP or LDF of the invention comprise at least one solvent selected from water (SOL1) and organic solvents. In one embodiment, organic solvents are comprised in amounts of about 30% to 60% by weight, relative to the total weight of the LEP.

In one embodiment, LEP or LDF comprise at least one organic solvent selected from monohydric alcohols (SOL2), dihydric alcohols, also called diols (SOL3), trihydric alcohols, also called triols (SOL4) and sugar alcohols (SOL5).

At least one monohydric alcohol (SOL2) is selected from C₂H₆O, 1-propanol, propan-2-ol, 1-butanol, 2-methyl-1-propanol, butan-2-ol, ethylene glycol methyl ether, ethylene glycol ethyl ether, ethylene glycol propyl ether, and ethylene glycol phenyl ether.

At least one dihydric alcohol (SOL3) is selected from butane-1,3-diol, pentane-1,4-diol, pentane-1,5-diol, pentane-2,4-diol, hexane-2,5-diol, vicinal diols (OH-groups at vicinal C; SOL3a) and alpha-omega diols (OH-groups located at one of the ends of a linear molecule (HO—R—OH), SOL3b).

In one embodiment, LEP or LDF comprise at least one vicinal diol (SOL3a) preferably selected from ethan-1,2-diol, propane-1,2-diol, butane-1,2-diol, butane-2,3-diol, pentane-1,2-diol, pentane-2,3-diol, hexane-2,3-diol, hexane-3,4-diol, heptane-1,2-diol, heptane-2,3-diol, heptane-3,4-diol, octane-1,2-diol, octane-2,3-diol, octane-3,4-diol, and octane-4,5-diol.

In one embodiment, LEP or LDF comprise at least one alpha-omega diol (SOL3b) preferably selected from, butane-1,4-diol, hexane-1,6-diol, propane-1,3-diol, 2-(2-hydroxyethoxy) ethanol, 2-(2-propoxyethoxy) ethanol, 2-(2-butoxyethoxy) ethanol and 2-methyl-2,4-pentandiol.

As a trihydric alcohol (SOL4) propane-1,2,3-triol may be comprised.

LEP, in one embodiment, comprise at least one sugar alcohol (alditol, SOL5) such as sorbitol, mannitol and erythriol, with sorbitol being preferred.

In one embodiment, LF1 to LF527 comprise SOL2 (preferably ethylene glycol phenyl ether), SOL3a (preferably propane-1,2-diol), SOL3b, SOL4 or SOL5 (preferably sorbitol). In one embodiment, LF1 to LF527 comprise a mixture of two solvents selected from SOL2 (preferably ethylene glycol phenyl ether), SOL3a (preferably propane-1,2-diol), SOL3b, SOL4 and SOL5 (preferably sorbitol). Preferably, at least one serine protease EPr9iA in LF82 to LF90 has at least the substitution R101E or R101D or R101S, preferably R101E (according to BPN′ numbering). Preferably, at least one serine protease EPr9iA in LF451 to LF469, LF479 and LF489 has at least the substitution R101E or R101D or R101S, preferably R101E (according to BPN′ numbering).

In one embodiment, the LEP comprises 0.5% to 15%, 1% to 15%, 2% to 15%, 0.5% to 10%, 0.5% to 8%, 0.5% to 6%, or 2% to 6% by weight of at least one hydrolase.

In one embodiment, the LEP comprises with increasing preference 5% to 70%, 10% to 70%, 20% to 70%, 30% to 70%, 40% to 70%, 10% to 60%, 20% to 60%, 30% to 60%, 40% to 60%, 10% to 55%, 10% to 50%, 20% to 50%, 30% to 50% or 40% to 50% by weight of at least one compound according to formula (I) as described herein.

In one embodiment, LEP comprise
a. 0.5% to 15% by weight of at least one hydrolase (EC 3) as disclosed above
b. 2% to 70% by weight, preferably 10% to 50% by weight, of at least one compound according to formula (I) as described herein, and
   about 30% of at least one solvent selected from SOL3a (preferably propane-1,2-diol), SOL4 and SOL5 (preferably sorbitol).

In another embodiment, LEP comprise
a. 0.5% to 15% by weight of at least one hydrolase (EC 3) as disclosed above
b. 2% to 70% by weight, preferably 10% to 50% by weight, of at least one compound according to formula (I) as described herein,
c. Water in amounts not exceeding 15% by weight, and
d. about 30% of at least one solvent selected from SOL3a (preferably propane-1,2-diol), SOL4 and SOL5 (preferably sorbitol).

In one embodiment, LEP may comprise at least two solvents selected from SOL3a (preferably propane-1,2-diol), SOL4 and SOL5 (preferably sorbitol), wherein the weight ratio of SOL3a:SOL4 or SOL3a:SOL5 is 2:1.

In one embodiment, LEP comprise the component according to formula (I) as described herein as organic solvent and no further organic solvent as disclosed above.

Thus, in a preferred embodiment the invention relates to liquid enzyme preparations (LEP) comprising
a. 0.5% to 15% by weight of at least one hydrolase (EC 3), preferably selected from protease, amylase, and lipase, most preferably a protease as described herein, and
b. 2% to 70% by weight, preferably 20%-60% by weight, of at least one compound according to formula (I)

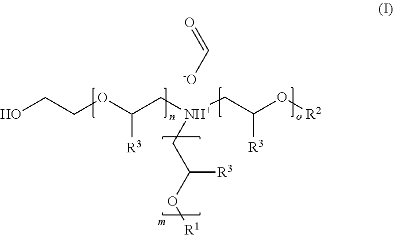

wherein R1 and R2 are selected from H and C₂H₄OH,
each of R3 is independently selected from H, methyl and ethyl, preferably all R3 are either H or methyl, and m, n, o are each individually 0-2, preferably 0-1, more preferably 0; preferably wherein compound (b) is triethanolamine formate,
wherein the amount of hydrolase refers to 100% active hydrolase; and
preferably water in amounts not exceeding 15% by weight and
preferably about 30% of at least one solvent selected from SOL3a (preferably propane-1,2-diol), SOL4 and SOL5 (preferably sorbitol);
and preferably further comprises at least one salt selected from a salt of a monovalent cation and a monovalent anion of 1-6 carbons, NaCl, KCl, CaCl2 and Na2SO4
wherein the LEP is preferably devoid of a surface-active anionic compound and a complexing anionic compound; and
wherein the LEP is preferably devoid of an enzyme inhibitor, preferably devoid of boron-containing compounds and peptide stabilizers.

LEP as described above can be prepared according to the following method: mixing at least one hydrolase (EC 3) with at least one compound according to formula (I) as described herein.

In a preferred embodiment, the method for preparing LEP comprises mixing the at least one hydrolase with at least one compound according to formula (I) as described herein, wherein the at least one hydrolase is comprised in a liquid enzyme concentrate prior to mixing with the at least one compound according to formula (I) as described herein, wherein the liquid enzyme concentrate preferably originates from fermentative enzyme production.

In a preferred embodiment, the at least one hydrolase is dissolved in a solvent selected from water and organic solvent, preferably water, prior to mixing with at least one compound according to formula (I) as described herein.

In a preferred embodiment, at least one compound according to formula (I) as described herein is used to provide a liquid enzyme preparation, which is homogeneous in its appearance and increased in stability of at least one hydrolase when compared to a liquid enzyme preparation lacking the compound according to formula (I) as described herein.

In a preferred embodiment, one compound according to formula (I) as described herein comprised in LEP or LDF is triethanolamine formate.

### Component (C)—Anionic Compounds

LDF in comparison to LEP additionally comprise at least 5% of at least one anionic compound. Thus, LDF according to the invention comprise:
(A) 0.0005% to 0.4% by weight of at least one hydrolase (EC 3)
(B) 4% to 20% by weight of a compound according to formula

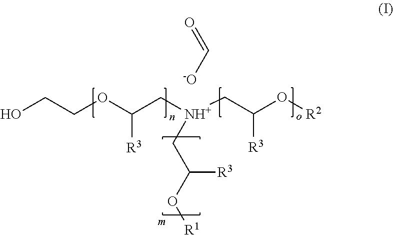

wherein R1 and R2 are selected from H and C₂H₄OH,
each of R3 is independently selected from H, methyl and ethyl, preferably all R3 are either H or methyl,
and m, n, o are each individually 0-2, preferably 0-1, more preferably 0; and
(C) at least 5% of at least one anionic compound.

Preferably, said LDF comprises 0.001% to 0.4%, 0.01% to 0.2%, or 0.01% to 0.1% by weight of at least one hydrolase.

Preferably, said LDF comprises 5% to 20%, 6% to 20%, 5% to 18%, or 5% to 16% by weight of at least one compound according to formula (I) as described herein.

Preferably, said LDF comprises 5% to 80%, 10% to 70%, 20% to 60%, or 30% to 50% by weight of at least one anionic compound.

Preferably, said LDF comprises water in amounts less than 80%, preferably less than 60-70% by weight, more preferably less than 50% by weight, all relative to the total weight of the detergent formulation.

Said LDF usually also comprise at least one compound selected from rheology modifiers, fragrances and colorants.

LDF can be prepared according to the following method: mixing at least one hydrolase (EC 3) with at least one compound according to formula (I) as described herein and at least one anionic compound in one or more steps, wherein the at least one hydrolase preferably is comprised in a liquid enzyme preparation prior to mixing with the at least one anionic compound.

In a preferred embodiment, at least one compound according to formula (I) as described herein is used to stabilize at least one hydrolase comprised in a liquid detergent formulation comprising at least one anionic compound selected from surface active anionic compounds and complexing anionic compounds or to provide a liquid detergent formulation, which is homogeneous in its appearance and with increased stability of at least one hydrolase when compared to a liquid detergent formulation lacking the compound according to formula (I).

In a preferred embodiment, a method to improve detergency of a liquid detergent formulation by the step of adding at least one compound according to formula (I) as described herein to a hydrolase-containing liquid detergent formulation is described, wherein detergency preferably is improved towards at least one stain selected from protease-sensitive stains, amylase-sensitive stains and lipase-sensitive stains.

The detergent composition can be a combination of liquid and solid detergent compositions.

The liquid detergent composition can be a gel detergent composition.

The detergent composition can be a unit dose or multi dose composition. The detergent composition can be in the form of a pouch, including multi-compartment pouches. The detergent composition can be a laundry or hard surface cleaning composition suitable for home care and/or industrial and institutional (I&I) cleaning. Preferably, the hard surface cleaning composition can be a dish washing detergent composition. The hard surface cleaning composition can be a medical cleaning composition, preferably a medical device cleaning composition. The hard surface cleaning composition can be an agrochemical device cleaning composition, preferably a spray tank cleaning composition. Both laundry and dish wash composition can be in the form of a hand wash or automated wash composition.

Thus, the present invention therefore also refers to a method for cleaning, preferably laundry or hard surface cleaning, comprising the step of contacting a subject, preferably a textile or a hard surface, with a composition comprising a composition as described herein, preferably wherein the composition comprises at least one additional detergent component, preferably a surfactant and/or a builder.

Anionic compounds according to the present invention include, but are not limited to, surface-active anionic compounds (also referred to as anionic surfactant, C1) and complexing anionic compounds (also referred to as builders, C2).

### Component (C1)—Surface-Active Anionic Compound

Anionic surfactants herein include, but are not limited to, surface-active compounds that contain a hydrophobic group and at least one water-solubilizing anionic group, usually selected from sulfates, sulfonate, and carboxylates to form a water-soluble compound.

### AS1

In one embodiment, LDF comprise at least one anionic surfactant selected from compounds of the general formula (AS1):

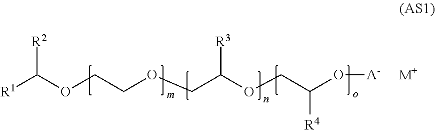

The variables in general formula (AS1) are defined as follows:
R¹ is selected from C₁-C₂₃-alkyl and C₂-C₂₃-alkenyl, wherein alkyl and/or alkenyl are linear (straight-chain; n-) or branched; examples are n-C₇H₁₅, n-C₉H₁₉, n-C₁₁H₂₃, n-C₁₂H₂₅, n-C₁₃H₂₇, n-C₁₄H₂₉, n-C₁₅H₃₁, n-C₁₆H₃₃, n-C₁₇H₃₅, n-C₁₈H₃₇, i-C₉H₁₉, i-C₁₂H₂₅.
R² is selected from H, C₁-C₂₀-alkyl and C₂-C₂₀-alkenyl, wherein alkyl and/or alkenyl are linear (straight-chain; n-) or branched.
R³ and R⁴, each independently selected from C₁-C₁₆-alkyl, wherein alkyl is linear (straight-chain; n-) or branched; examples are methyl, ethyl, n-propyl, isopropyl, n-butyl, isobutyl, secbutyl, tert-butyl, n-pentyl, isopentyl, sec-pentyl, neopentyl, 1,2-dimethylpropyl, isoamyl, n-hexyl, isohexyl, sec-hexyl, n-heptyl, n-octyl, 2-ethylhexyl, n-nonyl, n-decyl, isodecyl.
A⁻ is selected from —RCOO⁻, —SO₃⁻ and —RSO₃⁻, wherein R is selected from linear (straight-chain; n-) or branched C₁-C₆-alkyl, and C₁-C₄ hydroxyalkyl. Compounds might be called (fatty) alcohol/alkyl (ethoxy/ether) sulfates [(F)A(E)S] when A is —SO₃⁻, (fatty) alcohol/alkyl (ethoxy/ether) carboxylate [(F)A(E)C] when A is —RCOO⁻.
M⁺ is selected from H and salt forming cations. Salt forming cations may be monovalent or multivalent; hence M⁺ equals 1/v M^{v+}. Examples include but are not limited to sodium, potassium, magnesium, calcium, ammonium, and the ammonium salt of mono-, di, and triethanolamine.

The integers of the general formulae (AS1) are defined as follows:
m is in the range of zero to 200, preferably 1-80, more preferably 3-20; n and o, each independently in the range of zero to 100; n preferably is in the range of 0 to 10, more preferably 1 to 6; o preferably is in the range of 1 to 50, more preferably 4 to 25. The sum of m, n and o is at least one, preferably the sum of m, n and o is in the range of 5 to 100, more preferably in the range of from 9 to 50.

Anionic surfactants of the general formulae (AS1) may be of any structure, block copolymers or random copolymers.

In one embodiment, LDF comprise at least one anionic surfactant according to formula (AS1), wherein R¹ is n-C₁₀ to n-C₁₈, R² is H, A is SO₃, m, n and o being 0. M⁺ preferably is Na⁺. Such compounds may be called secondary alkane sulfonates (SAS) or paraffin sulfonates herein.

In one embodiment, LDF comprise at least one anionic surfactant according to formula (AS1), wherein R¹ is n-C₁₁H₂₃, R² is H, A is SO₃, m, n and o being 0. M⁺ preferably is NH₄⁺. Such compounds may be called ammonium lauryl sulfate (ALS) or AS1a herein.

In one embodiment, LDF comprise at least one anionic surfactant according to formula (AS1), wherein R¹ is n-C₁₁H₂₃, R² is selected from H, A is SO₃, m being 2-5, preferably 3, and n and o being 0. M⁺ preferably is Nat. Such compounds, herein, may be called laurylethersulfates (LES) or sodium laurylethersulfates (SLES) or AS1b.

Further suitable anionic surfactants include salts of C₁₂-C₁₈ sulfo fatty acid alkyl esters (such as C₁₂-C₁₈ sulfo fatty acid methyl esters), C₁₀-C₁₈-alkylarylsulfonic acids (such as n-C₁₀-C₁₈-alkylbenzene sulfonic acids) and C₁₀-C₁₈ alkyl alkoxy carboxylates.

In one embodiment, LDF comprise at least two anionic surfactants, both selected from compounds of general formula (AS1a), wherein one of said anionic surfactants is characterized in R¹ being C₁₁, R² being H, m being 2, n and o=0, A⁻ being SO₃⁻, salt forming cation (M⁺) being Na⁺ and the other surfactant is characterized in R¹ being C₁₃, R² being H, m being 2, n and o=0, A⁻ being SO₃⁻, M⁺ being Nat. Said anionic surfactant may be called AS1c herein.

### AS2

In one embodiment, LDF comprise at least one anionic surfactant selected from compounds of the general formula (AS2):

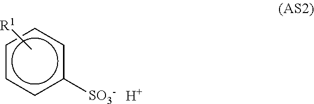

wherein R¹ in formula (AS2) is C₁₀-C₁₆ alkyl. LDF may comprise salts of compounds according to formula (AS2), preferably sodium salts.

In one embodiment, compounds according to formulas (AS2) means phenylalkane sulfonates with R¹ being C₁₂-, C₁₃-, C₁₄-, or C₁₆-alkyl.

In one embodiment, LDF comprise alkylbenzene sulfonates. In one aspect this means a compound according to formula (AS2) with R¹ being branched C₁₂ alkyl, which may be called BABS herein. In one aspect this means compounds according to formula (AS2) with R¹ being linear C₁₂ alkyl, which may be called AS2a herein.

In one embodiment, LDF comprise at least two anionic surfactants, both selected from compounds of general formula (AS2), wherein one of said anionic surfactants is characterized in R¹ being C₁₀, and the other surfactant is characterized in R¹ being C₁₃. Said combination may be called AS2b herein. In one embodiment, said the sodium salt of said compound is comprised in LDF and called AS2c herein.

### AS3

In one embodiment, LDF comprise at least one anionic surfactant selected from compounds of the general formula (AS3), which might be called N-acyl amino acid surfactants:

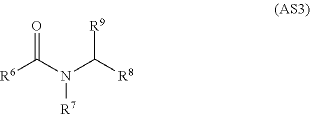

The variables in general formula (AS3) are defined as follows:
R⁶ is selected from linear (straight-chain; n-) or branched C6-C₂₂-alkyl and linear (straight-chain; n-) or branched C6-C₂₂-alkenyl such as oleyl.
R⁷ is selected from H and C₁-C₄-alkyl.
R⁸ is selected from H, methyl, —(CH₂)₃NHC(NH)NH₂, —CH₂C(O)NH₂, —CH₂C(O)OH, —(CH₂)₂C(O)NH₂, —(CH₂)₂C(O)OH, (imidazole-4-yl)-methyl, —CH(CH₃)C₂H₅, —CH₂CH(CH₃)₂, (CH₂)₄NH₂, benzyl, hydroxymethyl, —CH(OH)CH₃, (indole-3-yl)-methyl, (4-hydroxy-phenyl)methyl, isopropyl, —(CH₂)₂SCH₃, and —CH₂SH.
R⁹ is selected from —COOX and —CH₂SO₃X, wherein X is selected from Li⁺, Na⁺ and K⁺. Non-limiting examples of suitable N-acyl amino acid surfactants are the mono- and dicarboxylate salts (e.g. sodium, potassium, ammonium and ammonium salt of mono-, di, and triethanolamine) of N-acylated glutamic acid, for example, sodium cocoyl glutamate, sodium lauroyl glutamate, sodium myristoyl glutamate, sodium palmitoyl glutamate, sodium stearoyl glutamate, disodium cocoyl glutamate, disodium stearoyl glutamate, potassium cocoyl glutamate, potassium lauroyl glutamate, and potassium myristoyl glutamate; the carboxylate salts (e.g. sodium, potassium, ammonium and ammonium salt of mono-, di, and triethanolamine) of N-acylated alanine, for example, sodium cocoyl alaninate, and triethanolamine lauroyl alaninate; the carboxylate salts (e.g. sodium, potassium, ammonium and ammonium salt of mono-, di, and triethanolamine) of N-acylated glycine, for example, sodium cocoyl glycinate, and potassium cocoyl glycinate; the carboxylate salts (e.g. sodium, potassium, ammonium and ammonium salt of mono-, di, and triethanolamine) of N-acylated sarcosine, for example, sodium lauroyl sarcosinate, sodium cocoyl sarcosinate, sodium myristoyl sarcosinate, sodium oleoyl sarcosinate, and ammonium lauroyl sarcosinate.

### AS4

In one embodiment, LDF comprise at least one anionic surfactant selected from the group of soaps (AS4). In one embodiment, soaps are selected from salts of saturated and unsaturated C₁₂-C₁₈ fatty acids, such as lauric acid, myristic acid, palmitic acid, stearic acid, behenic acid, oleic acid, (hydrated) erucic acid. Salt forming cations (M⁺) may be monovalent or multivalent; hence M⁺ equals 1/v M^{v+}. Examples include but are not limited to sodium, potassium, magnesium, calcium, ammonium, and the ammonium salt of mono-, di, and triethanolamine.

Further non-limiting examples of suitable soaps include soap mixtures derived from natural fatty acids such as tallow, coconut oil, palm kernel oil, laurel oil, olive oil, or canola oil. Such soap mixtures comprise soaps of lauric acid and/or myristic acid and/or palmitic acid and/or stearic acid and/or oleic acid and/or linoleic acid in different amounts, depending on the natural fatty acids from which the soaps are derived.

Further non-limiting examples of suitable anionic surfactants include salts of sulfates, sulfonates or carboxylates derived from natural fatty acids such as tallow, coconut oil, palm kernel oil, laurel oil, olive oil, or canola oil. Such anionic surfactants comprise sulfates, sulfonates, or carboxylates of lauric acid and/or myristic acid and/or palmitic acid and/or stearic acid and/or oleic acid and/or linoleic acid in different amounts, depending on the natural fatty acids from which the soaps are derived.

### A+B+C1

In one embodiment, LDF of the invention comprise
at least one EPr9, preferably at least one EPr9i, more preferably at least one EPr9iA,
at least one AS1, preferably selected from AS1a, AS1b, and AS1c, and
at least one AAF selected from AAF1, AAF2, and AAF3, preferably selected from AAF1a, AAF2a and AAF3a.

In one embodiment, LDF of the invention comprise
at least one EPr9, preferably at least one EPr9i, more preferably at least one EPr9iA,
at least one AS2, preferably selected from AS2a, AS2b, and AS2c, and
at least one AAF selected from AAF1, AAF2, and AAF3, preferably selected from AAF1a, AAF2a and AAF3a.

In one embodiment, LDF of the invention comprise
at least one EPr9, preferably at least one EPr9i, more preferably at least one EPr9iA,
at least one AS3 and
at least one AAF selected from AAF1, AAF2, and AAF3, preferably selected from AAF1a, AAF2a and AAF3a.

In one embodiment, LDF of the invention comprise
at least one EPr9, preferably at least one EPr9i, more preferably at least one EPr9iA,
at least one AS4 and
at least one AAF selected from AAF1, AAF2, and AAF3, selected from AAF1a, AAF2a, and AAF3a.

In one embodiment, LDF of the invention comprise
at least one EPr9, preferably at least one EPr9i, more preferably at least one EPr9iA,
at least two different anionic surfactants and
at least one AAF selected from AAF1, AAF2, and AAF3, selected from AAF1a, AAF2a, and AAF3a.

Preferably, at least two different anionic surfactants are selected from AS1c, AS2b, and AS4.

In one embodiment, LDF of the invention comprise
at least one EPr9, preferably at least one EPr9i, more preferably at least one EPr9iA,
at least one AS1, preferably selected from AS1a, AS1b, and AS1c, and
at least one AAF selected from AAF1b, AAF2b, and AAF3b.

In one embodiment, LDF of the invention comprise
at least one EPr9, preferably at least one EPr9i, more preferably at least one EPr9iA,
at least one AS2, preferably selected from AS2a, AS2b, and AS2c, and
at least one AAF selected from AAF1b, AAF2b, and AAF3b.

In one embodiment, LDF of the invention comprise
at least one EPr9, preferably at least one EPr9i, more preferably at least one EPr9iA,
at least one AS3 and
at least one AAF selected from AAF1b, AAF2b, and AAF3b.

In one embodiment, LDF of the invention comprise
at least one EPr9, preferably at least one EPr9i, more preferably at least one EPr9iA,
at least one AS4 and
at least one AAF selected from AAF1b, AAF2b, and AAF3b.

In one embodiment, LDF of the invention comprise
at least one EPr9, preferably at least one EPr9i, more preferably at least one EPr9iA,
at least two different anionic surfactants and
at least one AAF selected from AAF1b, AAF2b, and AAF3b.

Preferably, at least two different anionic surfactants are selected from AS1c, AS2b, and AS4.

In one embodiment, LDF of the invention comprise
at least one EPr9, preferably at least one EPr9i, more preferably at least one EPr9iA,
at least one AS1, preferably selected from AS1a, AS1b, and AS1c, and
at least one AAF selected from AAF1c, AAF2c, and AAF3c.

In one embodiment, LDF of the invention comprise
at least one EPr9, preferably at least one EPr9i, more preferably at least one EPr9iA,
at least one AS2, preferably selected from AS2a, AS2b, and AS2c, and
at least one AAF selected from AAF1c, AAF2c, and AAF3c.

In one embodiment, LDF of the invention comprise
at least one EPr9, preferably at least one EPr9i, more preferably at least one EPr9iA,
at least one AS3 and
at least one AAF selected from AAF1c, AAF2c, and AAF3c.

In one embodiment, LDF of the invention comprise
at least one EPr9, preferably at least one EPr9i, more preferably at least one EPr9iA,
at least one AS4 and
at least one AAF selected from AAF1c, AAF2c, and AAF3c.

In one embodiment, LDF of the invention comprise
at least one EPr9, preferably at least one EPr9i, more preferably at least one EPr9iA,
at least two different anionic surfactants and
at least one AAF selected from AAF1c, AAF2c, and AAF3c.

Preferably, at least two different anionic surfactants are selected from AS1c, AS2b, and AS4. Specifically, LDF may comprise one of the following combinations (LDF):

| id | actual combinations |
|---|---|
| LDF1 | EPr9i + AS1 + AS2 + AAF1a |
| LDF2 | EPr9i + AS1 + AS3 + AAF1a |
| LDF3 | EPr9i + AS1a + AS4 + AAF1a |
| LDF4 | EPr9i + AS2 + AS3 + AAF1a |
| LDF5 | EPr9i + AS2 + AS4 + AAF1a |
| LDF6 | EPr9i + AS3 + AS4 + AAF1a |
| LDF7 | EPr9i + AS1a + AS1b + AAF1a |
| LDF8 | EPr9i + AS1a + AS1c + AAF1a |
| LDF9 | EPr9i + AS1b + AS1c + AAF1a |
| LDF10 | EPr9i + AS2a + AS2b + AAF1a |
| LDF11 | EPr9i + AS2a + AS2c + AAF1a |
| LDF12 | EPr9i + AS2b + AS2c + AAF1a |
| LDF13 | EPr9i + AS1a + AS2a + AS3 + AAF1a |
| LDF14 | EPr9i + AS1a + AS2a + AS4 + AAF1a |
| LDF15 | EPr9i + AS1a + AS2b + AS3 + AAF1a |
| LDF16 | EPr9i + AS1a + AS2b + AS4 + AAF1a |
| LDF17 | EPr9i + AS1a + AS2c + AS3 + AAF1a |
| LDF18 | EPr9i + AS1a + AS2c + AS4 + AAF1a |
| LDF19 | EPr9i + AS1b + AS2a + AS3 + AAF1a |
| LDF20 | EPr9i + AS1b + AS2a + AS4 + AAF1a |
| LDF21 | EPr9i + AS1b + AS2b + AS3 + AAF1a |
| LDF22 | EPr9i + AS1b + AS2b + AS4 + AAF1a |
| LDF23 | EPr9i + AS1b + AS2c + AS3 + AAF1a |
| LDF24 | EPr9i + AS1b + AS2c + AS4 + AAF1a |
| LDF25 | EPr9i + AS1c + AS2a + AS3 + AAF1a |
| LDF26 | EPr9i + AS1c + AS2a + AS4 + AAF1a |
| LDF27 | EPr9i + AS1c + AS2b + AS3 + AAF1a |
| LDF28 | EPr9i + AS1c + AS2b + AS4 + AAF1a |
| LDF29 | EPr9i + AS1c + AS2c + AS3 + AAF1a |
| LDF30 | EPr9i + AS1c + AS2c + AS4 + AAF1a |
| LDF31 | EPr9i + AS1a + AS2 + AAF2a |
| LDF32 | EPr9i + AS1a + AS3 + AAF2a |
| LDF33 | EPr9i + AS1a + AS4 + AAF2a |
| LDF34 | EPr9i + AS2 + AS3 + AAF2a |
| LDF35 | EPr9i + AS2 + AS4 + AAF2a |
| LDF36 | EPr9i + AS3 + AS4 + AAF2a |
| LDF37 | EPr9i + AS1a + AS1b + AAF2a |
| LDF38 | EPr9i + AS1a + AS1c + AAF2a |
| LDF39 | EPr9i + AS1b + AS1c + AAF2a |
| LDF40 | EPr9i + AS2a + AS2b + AAF2a |
| LDF41 | EPr9i + AS2a + AS2c + AAF2a |
| LDF42 | EPr9i + AS2b + AS2c + AAF2a |
| LDF43 | EPr9i + AS1a + AS2a + AS3 + AAF2a |
| LDF44 | EPr9i + AS1a + AS2a + AS4 + AAF2a |
| LDF45 | EPr9i + AS1a + AS2b + AS3 + AAF2a |
| LDF46 | EPr9i + AS1a + AS2b + AS4 + AAF2a |
| LDF47 | EPr9i + AS1a + AS2c + AS3 + AAF2a |
| LDF48 | EPr9i + AS1a + AS2c + AS4 + AAF2a |
| LDF49 | EPr9i + AS1b + AS2a + AS3 + AAF2a |
| LDF50 | EPr9i + AS1b + AS2a + AS4 + AAF2a |
| LDF51 | EPr9i + AS1b + AS2b + AS3 + AAF2a |
| LDF52 | EPr9i + AS1b + AS2b + AS4 + AAF2a |
| LDF53 | EPr9i + AS1b + AS2c + AS3 + AAF2a |
| LDF54 | EPr9i + AS1b + AS2c + AS4 + AAF2a |
| LDF55 | EPr9i + AS1c + AS2a + AS3 + AAF2a |
| LDF56 | EPr9i + AS1c + AS2a + AS4 + AAF2a |
| LDF57 | EPr9i + AS1c + AS2b + AS3 + AAF2a |
| LDF58 | EPr9i + AS1c + AS2b + AS4 + AAF2a |
| LDF59 | EPr9i + AS1c + AS2c + AS3 + AAF2a |
| LDF60 | EPr9i + AS1c + AS2c + AS4 + AAF2a |
| LDF61 | EPr9i + AS1a + AS2 + AAF3a |
| LDF62 | EPr9i + AS1a + AS3 + AAF3a |
| LDF63 | EPr9i + AS1a + AS4 + AAF3a |
| LDF64 | EPr9i + AS2 + AS3 + AAF3a |
| LDF65 | EPr9i + AS2 + AS4 + AAF3a |
| LDF66 | EPr9i + AS3 + AS4 + AAF3a |
| LDF67 | EPr9i + AS1a + AS1b + AAF3a |
| LDF68 | EPr9i + AS1a + AS1c + AAF3a |
| LDF69 | EPr9i + AS1b + AS1c + AAF3a |
| LDF70 | EPr9i + AS2a + AS2b + AAF3a |
| LDF71 | EPr9i + AS2a + AS2c + AAF3a |
| LDF72 | EPr9i + AS2b + AS2c + AAF3a |
| LDF73 | EPr9i + AS1a + AS2a + AS3 + AAF3a |
| LDF74 | EPr9i + AS1a + AS2a + AS4 + AAF3a |
| LDF75 | EPr9i + AS1a + AS2b + AS3 + AAF3a |
| LDF76 | EPr9i + AS1a + AS2b + AS4 + AAF3a |
| LDF77 | EPr9i + AS1a + AS2c + AS3 + AAF3a |
| LDF78 | EPr9i + AS1a + AS2c + AS4 + AAF3a |
| LDF79 | EPr9i + AS1b + AS2a + AS3 + AAF3a |
| LDF80 | EPr9i + AS1b + AS2a + AS4 + AAF3a |
| LDF81 | EPr9i + AS1b + AS2b + AS3 + AAF3a |
| LDF82 | EPr9i + AS1b + AS2b + AS4 + AAF3a |
| LDF83 | EPr9i + AS1b + AS2c + AS3 + AAF3a |
| LDF84 | EPr9i + AS1b + AS2c + AS4 + AAF3a |
| LDF85 | EPr9i + AS1c + AS2a + AS3 + AAF3a |
| LDF86 | EPr9i + AS1c + AS2a + AS4 + AAF3a |
| LDF87 | EPr9i + AS1c + AS2b + AS3 + AAF3a |
| LDF88 | EPr9i + AS1c + AS2b + AS4 + AAF3a |
| LDF89 | EPr9i + AS1c + AS2c + AS3 + AAF3a |
| LDF90 | EPr9i + AS1c + AS2c + AS4 + AAF3a |

In one embodiment, EPr9i in LDF1 to LDF90 is EPr9iA having at least a substitution at position 101 (according to BPN′ numbering), preferably selected from R101E, R101D and R101S, preferably R101E.

In one embodiment, LDF comprise on of the following combinations:

| id | actual combinations |
|---|---|
| LDF91 | EPr9iA + PSB1 + AS1a + AS2a + AS3 + AAF3a |
| LDF92 | EPr9iA + PSB1 + AS1a + AS2a + AS4 + AAF3a |
| LDF93 | EPr9iA + PSB1 + AS1a + AS2b + AS3 + AAF3a |
| LDF94 | EPr9iA + PSB1 + AS1a + AS2b + AS4 + AAF3a |
| LDF95 | EPr9iA + PSB1 + AS1a + AS2c + AS3 + AAF3a |
| LDF96 | EPr9iA + PSB1 + AS1a + AS2c + AS4 + AAF3a |
| LDF97 | EPr9iA + PSB1 + AS1b + AS2a + AS3 + AAF3a |
| LDF98 | EPr9iA + PSB1 + AS1b + AS2a + AS4 + AAF3a |
| LDF99 | EPr9iA + PSB1 + AS1b + AS2b + AS3 + AAF3a |
| LDF100 | EPr9iA + PSB1 + AS1b + AS2b + AS4 + AAF3a |
| LDF101 | EPr9iA + PSB1 + AS1b + AS2c + AS3 + AAF3a |
| LDF102 | EPr9iA + PSB1 + AS1b + AS2c + AS4 + AAF3a |
| LDF103 | EPr9iA + PSB1 + AS1c + AS2a + AS3 + AAF3a |
| LDF104 | EPr9iA + PSB1 + AS1c + AS2a + AS4 + AAF3a |
| LDF105 | EPr9iA + PSB1 + AS1c + AS2b + AS3 + AAF3a |
| LDF106 | EPr9iA + PSB1 + AS1c + AS2b + AS4 + AAF3a |
| LDF107 | EPr9iA + PSB1 + AS1c + AS2c + AS3 + AAF3a |
| LDF108 | EPr9iA + PSB1 + AS1c + AS2c + AS4 + AAF3a |
| LDF109 | EPr9iA + PSP2 + AS1a + AS2a + AS3 + AAF3a |
| LDF110 | EPr9iA + PSP2 + AS1a + AS2a + AS4 + AAF3a |
| LDF111 | EPr9iA + PSP2 + AS1a + AS2b + AS3 + AAF3a |
| LDF112 | EPr9iA + PSP2 + AS1a + AS2b + AS4 + AAF3a |
| LDF113 | EPr9iA + PSP2 + AS1a + AS2c + AS3 + AAF3a |
| LDF114 | EPr9iA + PSP2 + AS1a + AS2c + AS4 + AAF3a |
| LDF115 | EPr9iA + PSP2 + AS1b + AS2a + AS3 + AAF3a |
| LDF116 | EPr9iA + PSP2 + AS1b + AS2a + AS4 + AAF3a |
| LDF117 | EPr9iA + PSP2 + AS1b + AS2b + AS3 + AAF3a |
| LDF118 | EPr9iA + PSP2 + AS1b + AS2b + AS4 + AAF3a |
| LDF119 | EPr9iA + PSP2 + AS1b + AS2c + AS3 + AAF3a |
| LDF120 | EPr9iA + PSP2 + AS1b + AS2c + AS4 + AAF3a |
| LDF121 | EPr9iA + PSP2 + AS1c + AS2a + AS3 + AAF3a |
| LDF122 | EPr9iA + PSP2 + AS1c + AS2a + AS4 + AAF3a |
| LDF123 | EPr9iA + PSP2 + AS1c + AS2b + AS3 + AAF3a |
| LDF124 | EPr9iA + PSP2 + AS1c + AS2b + AS4 + AAF3a |
| LDF125 | EPr9iA + PSP2 + AS1c + AS2c + AS3 + AAF3a |
| LDF126 | EPr9iA + PSP2 + AS1c + AS2c + AS4 + AAF3a |

In one embodiment, EPr9Ai in LDF91 to LDF126 has at least a substitution at position 101 (according to BPN′ numbering), preferably selected from R101E, R101D and R101S, preferably R101E.

In a preferred embodiment, LDF are essentially devoid of enzyme stabilizers selected from boron-containing stabilizers and peptide stabilizers. “Essentially devoid of enzyme stabilizers” means that the molar ratio of enzyme to enzyme stabilizer selected from boron-containing stabilizers and peptide stabilizers in liquid detergent formulation is at least higher than 1 more preferred higher than 5 and more preferably higher than 10.

### Component (C2)—Complexing Anionic Compound

In one embodiment LDF of the invention comprise
(A) 0.0005% to 0.4% by weight of at least one hydrolase (EC 3)
(B) 4% to 20% by weight of a compound according to formula (I)

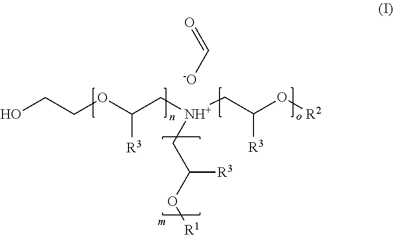

wherein R¹ and R² are selected from H and C2H₄OH, each of R³ is independently selected from H, methyl and ethyl, preferably all R³ are either H or methyl, and m, n, o are each individually 0-2, preferably 0-1, more preferably 0, and
(C) at least one complexing anionic compound (C2).

Complexing anionic compound (also called herein “builder” or “building agents”) herein means compounds selected from complexing agents (chelating agents, sequestrating agents), precipitating agents, and ion exchange compounds, which may form water-soluble complexes with calcium and magnesium. The term is not intended to limit such compounds to said function in the final application of the LDF.

Preferably, LDF disclosed herein are essentially devoid of phosphate-based builders (PBB). PBB include but are not limited to sodium metaphosphate, sodium orthophosphate, sodium hydrogenphosphate, sodium pyrophosphate, trisodium phosphate, hexasodium metaphosphate, and polyphosphates such as pentasodium tripolyphosphate (STP).

“Essentially devoid of PBB” means that no phosphate-based builder is added to LDF on purpose, preferably the PBB content in LDF is in total below 0.2% by weight, preferably not more than 10 ppm, determined by gravimetry. % by weight is relative to the total weight of the detergent formulation.

In one embodiment, LDF comprise at least one non-phosphate-based builder (NPB) which include sodium gluconate, citrate(s), silicate(s), carbonate(s), phosphonate(s), amino carboxylate(s), polycarboxylate(s), polysulfonate(s), and polyphosphonate(s).

### Citrates (NPB1)

In one embodiment, LDF comprise one or more citrates (NPB1). NPB1 include the mono- and the dialkali metal salts and in particular the mono- and preferably the trisodium salt of citric acid, ammonium or substituted ammonium salts of citric acid as well as citric acid as such. Citrate can be used as the anhydrous compound or as the hydrate, for example as sodium citrate dihydrate.

### Carbonates (NPB2)

In one embodiment, LDF comprise one or more carbonates (NPB2). NPB2 include alkali metal carbonates and alkali metal hydrogen carbonates, preferred are sodium salts. Particularly suitable is sodium carbonate (Na₂CO₃).

### Aminocarboxylates (NPB3)

In one embodiment, LDF comprise one or more aminocarboxylates (NPB3 or NPBAC). NPB3 or NPBAC include but are not limited to: diethanol glycine (DEG), dimethylglycine (DMG), nitrilitriacetic acid (NTA), N-hydroxyethylaminodiacetic acid, ethylenediaminetetraacetic acid (EDTA), N-(2hydroxyethyl)iminodiacetic acid (HEIDA), hydroxyethylenediaminetriacetic acid, N-hydroxyethyl-ethylenediaminetriacetic acid (HEDTA), hydroxyethylenediaminetetraacetic acid, diethylenetriaminepentaacetic acid (DTPA), and methylglycinediacetic acid (MGDA), glutamic acid-diacetic acid (GLDA), iminodisuccinic acid (IDS), hydroxyiminodisuccinic acid, ethylenediaminedisuccinic acid (EDDS), aspartic acid-diacetic acid, and alkali metal salts or ammonium salts thereof. Further suitable are aspartic acid-N-monoacetic acid (ASMA), aspartic acid-N,N-diacetic acid (ASDA), aspartic acid-N-monopropionic acid (ASMP), N-(2-sulfomethyl) aspartic acid (SMAS), N-(2-sulfoethyl) aspartic acid (SEAS), N-(2-sulfomethyl) glutamic acid (SMGL), N-(2-sulfoethyl) glutamic acid (SEGL), N-methyliminodiacetic acid (MIDA), alpha-alanine-N,N-diacetic acid (alpha-ALDA), serine-N,N-diacetic acid (SEDA), isoserine-N,N-diacetic acid (ISDA), phenylalanine-N,N-diacetic acid (PHDA), anthranilic acid-N,N-diacetic acid (ANDA), sulfanilic acid-N,N-diacetic acid (SLDA), taurine-N,N-diacetic acid (TUDA) and sulfomethyl-N,N-diacetic acid (SMDA) and alkali metal salts or ammonium salts thereof. The term “ammonium salts” as used in in this context refers to salts with at least one cation that bears a nitrogen atom that is permanently or temporarily quaternized. Examples of cations that bear at least one nitrogen atom that is permanently quaternized include tetramethylammonium, tetraethylammonium, dimethyldiethyl ammonium, and n-C₁₀-C₂₀-alkyl trimethyl ammonium. Examples of cations that bear at least one nitrogen atom that is temporarily quaternized include protonated amines and ammonia, such as monomethyl ammonium, dimethyl ammonium, trimethyl ammonium, monoethyl ammonium, diethyl ammonium, triethyl ammonium, n-C₁₀-C₂₀-alkyl dimethyl ammonium 2-hydroxyethylammonium, bis(2-hydroxyethyl) ammonium, tris(2-hydroxyethyl) ammonium, N-methyl 2-hydroxyethyl ammonium, N,N-dimethyl-2-hydroxyethylammonium, and especially NH₄⁺.

Preferably, LDF contain less than 0.2% by weight of nitrilotriacetic acid (NTA), or 0.01% to 0.1% by weight, all relative to the total weight of the detergent formulation.

In one embodiment, LDF comprise at least one compound selected from iminodisuccinic acid (IDS; NPBAC1), methylglycine diacetate (MGDA, NPBAC2), glutamic acid diacetate (GLDA, NPBAC3), and the respective salts thereof.

In one embodiment, LDF comprise at least one aminocarboxylate selected from methylglycine diacetate (MGDA), glutamic acid diacetate (GLDA), and the respective salts thereof, e.g. alkali (such as sodium) salts thereof in amounts in the range of 0.1% to 25.0% by weight, in the range of 1.0% to 18.0% by weight, in the range of 3.0% to 15.0% by weight, in the range of 3.0% to 10.0% by weight, or in the range of 5.0% to 8.0% by weight relative to the total weight of the detergent composition. Suitable salts of MGDA and of GLDA include the trialkali metal salts of MGDA (formula NPBAC2) and the tetraalkali metal salts of GLDA (formula NPBAC3):

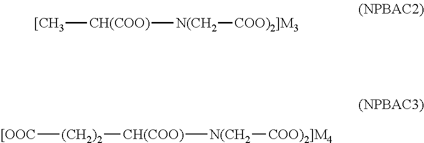

wherein the variables in formulae (NPBAC2) and (NPBAC3) are defined as follows: M is selected from alkali metal cations, same or different, for example cations of lithium, sodium, potassium, rubidium, cesium, and combinations of at least two of the foregoing. Preferred examples of alkali metal cations are sodium and potassium and combinations of sodium and potassium. More preferred are the sodium salts.

In one embodiment, alkali metal salts of MGDA are selected from [CH₃—CH(COO)—N(CH₂—COO)₂]Na_{3-y}H_{y} and [CH₃—CH(COO)—N(CH₂—COO)₂]Na_{3-x-y}(NH₄)ₓH_{y}, wherein:
x is selected from 0.0 to 1.0, preferably 0.1 to 0.5, more preferably 0.1 to 0.3;
y is selected from 0.0 to 1.0, preferably 0.0005 to 0.5.

Examples include Na_{3-y}H_{y}, [Na_{0.7}(NH₄)_{0.3}]_{3-y}H_{y}, [(NH₄)_{0.7}Na_{0.3}]_{3-y}H_{y}. Preferred examples are selected from Na_{3-y}H_{y}.

In one embodiment, MGDA is selected from at least one alkali metal salt of racemic MGDA and from alkali metal salts of mixtures of L- and D-enantiomers according to formula (NPBAC2), said mixture containing predominantly the respective L-isomer with an enantiomeric excess (ee) in the range of from 5 to 99%, preferably 5 to 95%, more preferably from 10 to 75% and even more preferably from 10 to 66%.

In one embodiment, the total degree of alkali neutralization of MGDA is in the range of from 0.80 to 0.98 mol-%, preferred are 0.90 to 0.97%. The total degree of alkali neutralization does not take into account any neutralization with ammonium.

In one embodiment, alkali metal salts of GLDA are selected from [OOC—(CH₂)₂—CH(COO)—N(CH₂—COO)₂]Na_{4-y}H_{y} and [OOC—(CH₂)₂—CH(COO)—N(CH₂—COO)₂]M_{4-x-y}(NH₄)_{y}Hₓ, wherein:
x is selected from 0.0 to 2.0, preferably from 0.02 to 0.5, more preferably from 0.1 to 0.3,
y is selected from 0.0 to 1.0, preferably from 0.0005 to 0.5.

In one embodiment, alkali metal salts of GLDA may be selected from alkali metal salts of the L- and D-enantiomers according to formula (NPBAC3), said mixture containing the racemic mixture or preferably predominantly the respective L-isomer, for example with an enantiomeric excess (ee) in the range of from 5 to 99%, preferably 5 to 95%.

The enantiomeric excess can be determined, e.g. by measuring the polarization (polarimetry) or preferably by chromatography, for example by HPLC with a chiral column, for example with one or more cyclodextrins as immobilized phase. Preferred is determination of the enantiomeric excess by HPLC with an immobilized optically active ammonium salt such as D-penicillamine.

Generally, in the context of this disclosure, small amounts of MGDA and/or GLDA may also bear a cation other than alkali metal. It is thus possible that small amounts of builder, such as 0.01% to 5 mol-% of total builder may bear alkali earth metal cations such as, e.g. Mg²⁺ or Ca²⁺, or a transition metal cation such as, e.g. a Fe²⁺ or Fe³⁺ cation. “Small amounts” of MGDA and/or GLDA herein refer to a total of 0.1% to 1 w/w %, relative to the respective builder.

In one embodiment, LDF of the invention comprise more than one aminocarboxylate (NPB3). Specifically, LDF may comprise one of the following combinations (C-NPBAC):

| id | actual combinations |
|---|---|
| C-NPBAC1 | NPBAC1 + NPBAC2 |
| C-NPBAC2 | NPBAC1 + NPBAC3 |
| C-NPBAC3 | NPBAC2 + NPBAC3 |
| C-NPBAC4 | NPBAC1 + NPBAC2 + NPBAC3 |

### A+B+C2

In one embodiment, LDF of the invention comprise
at least one EPr9, preferably at least one EPr9i, more preferably at least one EPr9iA,
at least one NPB1, preferably sodium citrate dihydrate, and
at least one AAF selected from AAF1, AAF2, and AAF3, preferably selected from AAF1a, AAF2a, and AAF3a.

In one embodiment, LDF of the invention comprise
at least one EPr9, preferably at least one EPr9i, more preferably at least one EPr9iA,
at least one NPB2, preferably Na₂CO₃, and
at least one AAF selected from AAF1, AAF2, and AAF3, preferably selected from AAF1a, AAF2a, and AAF3a.

In one embodiment, LDF of the invention comprise
at least one EPr9, preferably at least one EPr9i, more preferably at least one EPr9iA,
at least one NPB3, preferably selected from NPBAC1, NPBAC2 and NPBAC3, and
at least one AAF selected from AAF1, AAF2, and AAF3, preferably selected from AAF1a, AAF2a, and AAF3a.

In one embodiment, LDF of the invention comprise
at least one EPr9, preferably at least one EPr9i, more preferably at least one EPr9iA,
at least one selected from C-NPBAC1, C-NPBAC2, C-NPBAC3, and C-NPBAC4 and
at least one AAF selected from AAF1, AAF2, and AAF3, preferably selected from AAF1a, AAF2a, and AAF3a.

In one embodiment, LDF of the invention comprise
at least one EPr9, preferably at least one EPr9i, more preferably at least one EPr9iA,
at least one NPB1, preferably sodium citrate dihydrate, and
at least one AAF selected from AAF1b, AAF2b, and AAF3b.

In one embodiment, LDF of the invention comprise
at least one EPr9, preferably at least one EPr9i, more preferably at least one EPr9iA,
at least one NPB2, preferably Na₂CO₃, and
at least one AAF selected from AAF1b, AAF2b, and AAF3b.

In one embodiment, LDF of the invention comprise
at least one EPr9, preferably at least one EPr9i, more preferably at least one EPr9iA,
at least one NPB3, preferably selected from NPBAC₁, NPBAC₂, and NPBAC3, and
at least one AAF selected from AAF1b, AAF2b, and AAF3b.

In one embodiment, LDF of the invention comprise
at least one EPr9, preferably at least one EPr9i, more preferably at least one EPr9iA,
at least one selected from C-NPBAC1, C-NPBAC2, C-NPBAC3, and C-NPBAC4 and
at least one AAF selected from AAF1b, AAF2b, and AAF3b.

In one embodiment, LDF of the invention comprise
at least one EPr9, preferably at least one EPr9i, more preferably at least one EPr9iA,
at least one NPB1, preferably sodium citrate dihydrate, and
at least one AAF selected from AAF1c, AAF2c, and AAF3c.

In one embodiment, LDF of the invention comprise
at least one EPr9, preferably at least one EPr9i, more preferably at least one EPr9iA,
at least one NPB2, preferably Na₂CO₃, and
at least one AAF selected from AAF1c, AAF2c, and AAF3c.

In one embodiment, LDF of the invention comprise
at least one EPr9, preferably at least one EPr9i, more preferably at least one EPr9iA,
at least one NPB3, preferably selected from NPBAC₁, NPBAC₂, and NPBAC3, and
at least one AAF selected from AAF1c, AAF2c, and AAF3c.

In one embodiment, LDF of the invention comprise
at least one EPr9, preferably at least one EPr9i, more preferably at least one EPr9iA,
at least one selected from C-NPBAC1, C-NPBAC2, C-NPBAC3, and C-NPBAC4 and
at least one AAF selected from AAF1c, AAF2c, and AAF3c.

In one embodiment, one of the following combinations of builders (C-NPB) may be comprised in inventive LDF:

| id | actual combinations |
|---|---|
| C-NPB1 | NPB1 + NPB2 |
| C-NPB2 | NPB1 + NPBAC1 |
| C-NPB3 | NPB1 + NPBAC2 |
| C-NPB4 | NPB1 + NPBAC3 |
| C-NPB5 | NPB1 + C-NPBAC1 |
| C-NPB6 | NPB1 + C-NPBAC2 |
| C-NPB7 | NPB1 + C-NPBAC3 |
| C-NPB8 | NPB1 + C-NPBAC4 |
| C-NPB9 | NPB2 + NPBAC1 |
| C-NPB10 | NPB2 + NPBAC2 |
| C-NPB11 | NPB2 + NPBAC3 |
| C-NPB12 | NPB2 + C-NPBAC1 |
| C-NPB13 | NPB2 + C-NPBAC2 |
| C-NPB14 | NPB2 + C-NPBAC3 |
| C-NPB15 | NPB2 + C-NPBAC4 |
| C-NPB16 | NPB1 + NPB2 + NPBAC1 |
| C-NPB17 | NPB1 + NPB2 + NPBAC2 |
| C-NPB18 | NPB1 + NPB2 + NPBAC3 |
| C-NPB19 | NPB1 + NPB2 + C-NPBAC1 |
| C-NPB20 | NPB1 + NPB2 + C-NPBAC2 |
| C-NPB21 | NPB1 + NPB2 + C-NPBAC3 |
| C-NPB22 | NPB1 + NPB2 + C-NPBAC4 |

In one embodiment, LDF comprise
at least one EPr9, preferably at least one EPr9i, more preferably at least one EPr9iA,
at least one out of combinations C-NPB1 to C-NPB22, and
at least one AAF selected from AAF1, AAF2, and AAF3, preferably selected from AAF1a, AAF2a, and AAF3a.

In one embodiment, LDF comprise
at least one EPr9, preferably at least one EPr9i, more preferably at least one EPr9iA,
at least one out of combinations C-NPB1 to C-NPB22, and
at least one AAF selected from AAF1b, AAF2b, and AAF3b.

In one embodiment, LDF comprise
at least one EPr9, preferably at least one EPr9i, more preferably at least one EPr9iA,
at least one out of combinations C-NPB1 to C-NPB22, and
at least one AAF selected from AAF1c, AAF2c, and AAF3c.

Specifically, LDF may comprise one of the following combinations (LDF):

| id | actual combinations |
|---|---|
| LDF127 | EPr9i + NPB1 + AAF1a |
| LDF128 | EPr9i + NPB2 + AAF1a |
| LDF129 | EPr9i + NPBAC1 + AAF1a |
| LDF130 | EPr9i + NPBAC2 + AAF1a |
| LDF131 | EPr9i + NPBAC3 + AAF1a |
| LDF132 | EPr9i + C-NPB1 + AAF1a |
| LDF133 | EPr9i + C-NPB2 + AAF1a |
| LDF134 | EPr9i + C-NPB3 + AAF1a |
| LDF135 | EPr9i + C-NPB4 + AAF1a |
| LDF136 | EPr9i + C-NPB5 + AAF1a |
| LDF137 | EPr9i + C-NPB6 + AAF1a |
| LDF138 | EPr9i + C-NPB7 + AAF1a |
| LDF139 | EPr9i + C-NPB8 + AAF1a |
| LDF140 | EPr9i + C-NPB9 + AAF1a |
| LDF141 | EPr9i + C-NPB10 + AAF1a |
| LDF142 | EPr9i + C-NPB11 + AAF1a |
| LDF143 | EPr9i + C-NPB12 + AAF1a |
| LDF144 | EPr9i + C-NPB13 + AAF1a |
| LDF145 | EPr9i + C-NPB14 + AAF1a |
| LDF146 | EPr9i + C-NPB15 + AAF1a |
| LDF147 | EPr9i + C-NPB16 + AAF1a |
| LDF148 | EPr9i + C-NPB17 + AAF1a |
| LDF149 | EPr9i + C-NPB18 + AAF1a |
| LDF150 | EPr9i + C-NPB19 + AAF1a |
| LDF151 | EPr9i + C-NPB20 + AAF1a |
| LDF152 | EPr9i + C-NPB21 + AAF1a |
| LDF153 | EPr9i + C-NPB22 + AAF1a |
| LDF154 | EPr9i + NPB1 + AAF2a |
| LDF155 | EPr9i + NPB2 + AAF2a |
| LDF156 | EPr9i + NPBAC1 + AAF2a |
| LDF157 | EPr9i + NPBAC2 + AAF2a |
| LDF158 | EPr9i + NPBAC3 + AAF2a |
| LDF159 | EPr9i + C-NPB1 + AAF2a |
| LDF160 | EPr9i + C-NPB2 + AAF2a |
| LDF161 | EPr9i + C-NPB3 + AAF2a |
| LDF162 | EPr9i + C-NPB4 + AAF2a |
| LDF163 | EPr9i + C-NPB5 + AAF2a |
| LDF164 | EPr9i + C-NPB6 + AAF2a |
| LDF165 | EPr9i + C-NPB7 + AAF2a |
| LDF166 | EPr9i + C-NPB8 + AAF2a |
| LDF167 | EPr9i + C-NPB9 + AAF2a |
| LDF168 | EPr9i + C-NPB10 + AAF2a |
| LDF169 | EPr9i + C-NPB11 + AAF2a |
| LDF170 | EPr9i + C-NPB12 + AAF2a |
| LDF171 | EPr9i + C-NPB13 + AAF2a |
| LDF172 | EPr9i + C-NPB14 + AAF2a |
| LDF173 | EPr9i + C-NPB15 + AAF2a |
| LDF174 | EPr9i + C-NPB16 + AAF2a |
| LDF175 | EPr9i + C-NPB17 + AAF2a |
| LDF176 | EPr9i + C-NPB18 + AAF2a |
| LDF177 | EPr9i + C-NPB19 + AAF2a |
| LDF178 | EPr9i + C-NPB20 + AAF2a |
| LDF179 | EPr9i + C-NPB21 + AAF2a |
| LDF180 | EPr9i + C-NPB22 + AAF2a |
| LDF181 | EPr9i + NPB1 + AAF3a |
| LDF182 | EPr9i + NPB2 + AAF3a |
| LDF183 | EPr9i + NPBAC1 + AAF3a |
| LDF184 | EPr9i + NPBAC2 + AAF3a |
| LDF185 | EPr9i + NPBAC3 + AAF3a |
| LDF186 | EPr9i + C-NPB1 + AAF3a |
| LDF187 | EPr9i + C-NPB2 + AAF3a |
| LDF188 | EPr9i + C-NPB3 + AAF3a |
| LDF189 | EPr9i + C-NPB4 + AAF3a |
| LDF190 | EPr9i + C-NPB5 + AAF3a |
| LDF191 | EPr9i + C-NPB6 + AAF3a |
| LDF192 | EPr9i + C-NPB7 + AAF3a |
| LDF193 | EPr9i + C-NPB8 + AAF3a |
| LDF194 | EPr9i + C-NPB9 + AAF3a |
| LDF195 | EPr9i + C-NPB10 + AAF3a |
| LDF196 | EPr9i + C-NPB11 + AAF3a |
| LDF197 | EPr9i + C-NPB12 + AAF3a |
| LDF198 | EPr9i + C-NPB13 + AAF3a |
| LDF199 | EPr9i + C-NPB14 + AAF3a |
| LDF200 | EPr9i + C-NPB15 + AAF3a |
| LDF201 | EPr9i + C-NPB16 + AAF3a |
| LDF202 | EPr9i + C-NPB17 + AAF3a |
| LDF203 | EPr9i + C-NPB18 + AAF3a |
| LDF204 | EPr9i + C-NPB19 + AAF3a |
| LDF205 | EPr9i + C-NPB20 + AAF3a |
| LDF206 | EPr9i + C-NPB21 + AAF3a |
| LDF207 | EPr9i + C-NPB22 + AAF3a |

In one embodiment, EPr9i in LDF127 to LDF207 is EPr9iA having at least a substitution at position 101 (according to BPN′ numbering), preferably selected from R101E, R101D and R101S, preferably R101E.

In one embodiment, LDF comprise on of the following combinations:

| id | actual combinations |
|---|---|
| LDF208 | EPr9iA + PSB1 + NPB1 + AAF3a |
| LDF209 | EPr9iA + PSB1 + NPB2 + AAF3a |
| LDF210 | EPr9iA + PSB1 + NPBAC1 + AAF3a |
| LDF211 | EPr9iA + PSB1 + NPBAC2 + AAF3a |
| LDF212 | EPr9iA + PSB1 + NPBAC3 + AAF3a |
| LDF213 | EPr9iA + PSB1 + C-NPB1 + AAF3a |
| LDF214 | EPr9iA + PSB1 + C-NPB2 + AAF3a |
| LDF215 | EPr9iA + PSB1 + C-NPB3 + AAF3a |
| LDF216 | EPr9iA + PSB1 + C-NPB4 + AAF3a |
| LDF217 | EPr9iA + PSB1 + C-NPB5 + AAF3a |
| LDF218 | EPr9iA + PSB1 + C-NPB6 + AAF3a |
| LDF219 | EPr9iA + PSB1 + C-NPB7 + AAF3a |
| LDF220 | EPr9iA + PSB1 + C-NPB8 + AAF3a |
| LDF221 | EPr9iA + PSB1 + C-NPB9 + AAF3a |
| LDF222 | EPr9iA + PSB1 + C-NPB10 + AAF3a |
| LDF223 | EPr9iA + PSB1 + C-NPB11 + AAF3a |
| LDF224 | EPr9iA + PSB1 + C-NPB12 + AAF3a |
| LDF225 | EPr9iA + PSB1 + C-NPB13 + AAF3a |
| LDF226 | EPr9iA + PSB1 + C-NPB14 + AAF3a |
| LDF227 | EPr9iA + PSB1 + C-NPB15 + AAF3a |
| LDF228 | EPr9iA + PSB1 + C-NPB16 + AAF3a |
| LDF229 | EPr9iA + PSB1 + C-NPB17 + AAF3a |
| LDF230 | EPr9iA + PSB1 + C-NPB18 + AAF3a |
| LDF231 | EPr9iA + PSB1 + C-NPB19 + AAF3a |
| LDF232 | EPr9iA + PSB1 + C-NPB20 + AAF3a |
| LDF233 | EPr9iA + PSB1 + C-NPB21 + AAF3a |
| LDF234 | EPr9iA + PSB1 + C-NPB22 + AAF3a |
| LDF235 | EPr9iA + PSP2 + NPB1 + AAF3a |
| LDF236 | EPr9iA + PSP2 + NPB2 + AAF3a |
| LDF237 | EPr9iA + PSP2 + NPBAC1 + AAF3a |
| LDF238 | EPr9iA + PSP2 + NPBAC2 + AAF3a |
| LDF239 | EPr9iA + PSP2 + NPBAC3 + AAF3a |
| LDF240 | EPr9iA + PSP2 + C-NPB1 + AAF3a |
| LDF241 | EPr9iA + PSP2 + C-NPB2 + AAF3a |
| LDF242 | EPr9iA + PSP2 + C-NPB3 + AAF3a |
| LDF243 | EPr9iA + PSP2 + C-NPB4 + AAF3a |
| LDF244 | EPr9iA + PSP2 + C-NPB5 + AAF3a |
| LDF245 | EPr9iA + PSP2 + C-NPB6 + AAF3a |
| LDF246 | EPr9iA + PSP2 + C-NPB7 + AAF3a |
| LDF247 | EPr9iA + PSP2 + C-NPB8 + AAF3a |
| LDF248 | EPr9iA + PSP2 + C-NPB9 + AAF3a |
| LDF249 | EPr9iA + PSP2 + C-NPB10 + AAF3a |
| LDF250 | EPr9iA + PSP2 + C-NPB11 + AAF3a |
| LDF251 | EPr9iA + PSP2 + C-NPB12 + AAF3a |
| LDF252 | EPr9iA + PSP2 + C-NPB13 + AAF3a |
| LDF253 | EPr9iA + PSP2 + C-NPB14 + AAF3a |
| LDF254 | EPr9iA + PSP2 + C-NPB15 + AAF3a |
| LDF255 | EPr9iA + PSP2 + C-NPB16 + AAF3a |
| LDF256 | EPr9iA + PSP2 + C-NPB17 + AAF3a |
| LDF257 | EPr9iA + PSP2 + C-NPB18 + AAF3a |
| LDF258 | EPr9iA + PSP2 + C-NPB19 + AAF3a |
| LDF259 | EPr9iA + PSP2 + C-NPB20 + AAF3a |
| LDF260 | EPr9iA + PSP2 + C-NPB21 + AAF3a |
| LDF261 | EPr9iA + PSP2 + C-NPB22 + AAF3a |

In one embodiment, EPr9Ai in LDF208 to LDF261 has at least a substitution at position 101 (according to BPN′ numbering), preferably selected from R101E, R101D and R101S, preferably R101E.

### A+B+C1+C2

In one embodiment LDF of the invention comprise
(A) 0.0005% to 0.4% by weight of at least one hydrolase (EC 3)
(B) 4% to 20% by weight of a compound according to formula (I) as described herein and
(C) at least 5% anionic compounds, wherein the anionic compounds comprise at least one anionic surfactant (C1) and at least one builder (C2).

Components (A), (B), (C1) and (C2) are with preferences as disclosed above.

In one embodiment, LDF of the invention comprise at least one out of combinations LDF1 to LDF126, and at least one NPB1, preferably sodium citrate dihydrate.

In one embodiment, LDF of the invention comprise at least one out of combinations LDF1 to LDF126, and at least one NPB2, preferably Na₂CO₃.

In one embodiment, LDF of the invention comprise at least one out of combinations LDF1 to LDF126, and at least one NPB3, preferably selected from NPBAC1, NPBAC2 and NPBAC3.

In one embodiment, LDF of the invention comprise at least one out of combination LDF1 to LDF126, and at least one out of combinations C-NPBAC1, C-NPBAC2, C-NPBAC3 and CNPBAC4.

In one embodiment, LDF comprise at least one out of combinations LDF1 to LDF126, and at least one out of combinations C-NPB1-C-NPB22.

In one embodiment, LDF of the invention comprise LDF28 and at least one NPB1, preferably sodium citrate dihydrate.

In one embodiment, LDF of the invention comprise LDF58 and at least one NPB1, preferably sodium citrate dihydrate.

In one embodiment, LDF of the invention comprise LDF88 and at least one NPB1, preferably sodium citrate dihydrate.

In one embodiment, LDF of the invention comprise LDF28 and at least one NPB2, preferably Na₂CO₃.

In one embodiment, LDF of the invention comprise LDF58 and at least one NPB2, preferably Na₂CO₃.

In one embodiment, LDF of the invention comprise LDF88 and at least one NPB2, preferably Na₂CO₃.

In one embodiment, LDF of the invention comprise LDF28 and at least one NPB3, preferably selected from NPBAC1, NPBAC2, and NPBAC3.

In one embodiment, LDF of the invention comprise LDF58 and at least one NPB3, preferably selected from NPBAC1, NPBAC2, and NPBAC3.

In one embodiment, LDF of the invention comprise LDF88 and at least one NPB3, preferably selected from NPBAC1, NPBAC2, and NPBAC3.

In one embodiment, LDF of the invention comprise LDF28 and at least one at least one out of combinations C-NPBAC1, C-NPBAC2, C-NPBAC3, and C-NPBAC4.

In one embodiment, LDF of the invention comprise LDF58 and at least one at least one out of combinations C-NPBAC1, C-NPBAC2, C-NPBAC3, and C-NPBAC4.

In one embodiment, LDF of the invention comprise LDF88 and at least one out of combinations C-NPBAC1, C-NPBAC2, C-NPBAC3, and C-NPBAC4.

In one embodiment, LDF comprise a surface-active anionic compound that is preferably selected from LAS (linear alkylbenzene sulfonates) or AES (alkyl ether sulphates) and a complexing anionic compound, preferably selected from citrates (NPB1) and aminocarboxylates (NPB3).

Thus, preferably the LDF according to the invention comprise:
(A) 0.0005% to 0.4% by weight of at least one hydrolase (EC 3), preferably a protease as described herein,
(B) 4% to 20% by weight of a compound according to formula (I) as described herein, preferably triethanolamine formate; and
(C) at least 5% of at least one anionic compound, wherein the anionic compound is selected from a surface-active anionic compound and a complexing anionic compound, preferably wherein the surface-active anionic compound is selected from LAS or AES and preferably wherein the complexing anionic compound is selected from citrates (NPB1) and aminocarboxylates (NPB3);
wherein preferably the liquid detergent formulation comprises ≤3% by weight, preferably ≤2% by weight, sodium formate.

### Component (D)—Further Detergent Component

In one embodiment LDF of the invention comprise
(A) 0.0005% to 0.4% by weight of at least one hydrolase (EC 3)
(B) 4% to 20% by weight of a compound according to formula (I) as described herein and
(C) at least one anionic compound, wherein the anionic compound is selected from a surface-active anionic (C1) compound and a complexing anionic compound (C2), and
(D) at least one further detergent component.

Components (A), (B), (C1) and (C2) are as disclosed above with preferences as disclosed above.

At least one further detergent component may be selected from:
at least one non-ionic surfactant (D1)
at least one additional hydrolase selected from amylase, lipase, cellulase and mannanase (D2)
at least one solvent (D3)
at least one polymer (D4)
at least one antimicrobial (D5)

### Component D1—Non-Ionic Surfactant

In one embodiment, the LDF of the invention comprises
(A) 0.0005% to 0.4% by weight of at least one hydrolase (EC 3)
(B) 4% to 20% by weight of a compound according to formula (I) as described herein and
(C) at least one anionic compound, wherein the anionic compound is selected from a surface-active anionic (C1) compound and a complexing anionic compound (C2), and
(D) at least one non-ionic surfactant (D1).

### NIS1

In one embodiment, LDF comprise at least one non-ionic surfactant selected from compounds of the general formulae (NIS1a) and (NIS1b):

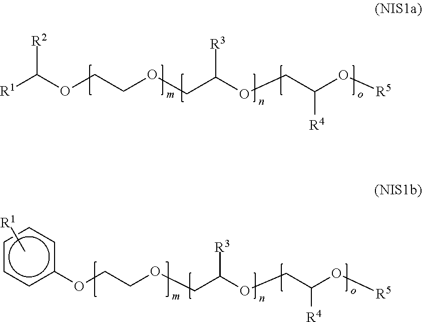

The variables of the general formulae (NIS1a) and (NIS1b) are defined as follows:
R¹ is selected from C₁-C₂₃ alkyl and C₂-C₂₃ alkenyl, wherein alkyl and/or alkenyl are linear (straight-chain; n-) or branched; examples are n-C₇H₁₅, n-C₈H₁₇, n-C₉H₁₉, n-C₁₁H₂₃, n-C₁₃H₂₇, n-C₁₅H₃₁, n-C₁₇H₃₅, i-C₉H₁₉, i-C₁₂H₂₅.
R² is selected from H, C₁-C₂₀ alkyl and C₂-C₂₀ alkenyl, wherein alkyl and/or alkenyl are linear (straight-chain; n-) or branched.
R³ and R⁴, each independently selected from C₁-C₁₆ alkyl, wherein alkyl is linear (straight-chain; n-) or branched; examples are methyl, ethyl, n-propyl, isopropyl, n-butyl, isobutyl, secbutyl, tert-butyl, n-pentyl, isopentyl, sec-pentyl, neopentyl, 1,2-dimethylpropyl, isoamyl, n-hexyl, isohexyl, sec-hexyl, n-heptyl, n-octyl, 2-ethylhexyl, n-nonyl, n-decyl, isodecyl.
R⁵ is selected from H and C₁-C₁₈ alkyl, wherein alkyl is linear (straight-chain; n-) or branched.

The integers of the general formulae (NIS1a) and (NIS1b) are defined as follows:
m is in the range of zero to 200, preferably 1-80, more preferably 3-20; n and o, each independently in the range of zero to 100; n preferably is in the range of 1 to 10, more preferably 1 to 6; o preferably is in the range of 1 to 50, more preferably 4 to 25. The sum of m, n and o is at least one, preferably the sum of m, n and o is in the range of 5 to 100, more preferably in the range of from 9 to 50.

Compounds according to formula (NIS1a) may be called alkyl polyethyleneglycol ether (AEO) herein. Compounds according to formula (NIS1b) may be called alkylphenol polyethyleneglycol ether (APEO) herein.

In one embodiment, detergent formulations comprise at least one non-ionic surfactant selected from compounds of the general formula (NIS1a) with R¹ being n-C₁₃H₂₇, R² and R⁵ being H, m being 3-20, n and o=0.

In one embodiment, detergent formulations comprise at least one non-ionic surfactant selected from compounds of the general formula (NIS1a) with R¹ being linear or branched C₁₀ alkyl, R² and R⁵ being H, m being 3-14, n and o=0.

In one embodiment, LDF comprise at least two non-ionic surfactants, both selected from compounds of the general formula (NIS1a), wherein one of said non-ionic surfactants is characterized in R¹ being n-C₁₅H₃₁, R² and R⁵ being H, m being 9-80, n and o=0, and the other surfactant is characterized in R¹ being n-C₁₇H₃₅, R² and R⁵ being H, m being 9-80, n and o=0. m in both non-ionic surfactants in one embodiment is 9-80, preferably 25-80. Said NIS may be called NIS1a1 herein.

In one embodiment, detergent formulations comprise at least one non-ionic surfactant selected from the general formula (NIS1a), wherein m is in the range of 3 to 11, preferably not more than 10, more preferably not more than 7; n and o being 0, R¹ being linear C₉-C₁₇ alkyl, R² and R⁵ being H.

In one embodiment, detergent formulations comprise at least two non-ionic surfactants, both selected from compounds of the general formula (NIS1a), wherein one of said non-ionic surfactants is characterized in R¹ being n-C₁₂H₂₅, R² and R⁵ being H, m being 3-30, preferably 7, n and o=0, and the other surfactant is characterized in R¹ being n-C₁₄H₂₉, R² and R⁵ being H, m being 3-30, preferably 7, n and o=0. Said NIS may be called NIS1a2 herein.

In one embodiment, detergent formulations comprise at least two non-ionic surfactants, both selected from compounds of the general formula (NIS1a), wherein one of said non-ionic surfactants is characterized in R¹ being n-C₁₁H₂₃, R² and R⁵ being H, m being 4-10, n and o=0, and the other surfactant is characterized in R¹ selected from n-C₁₁H₂₃ and n-C₁₇H₃₅, R² and R⁵ being H, m being 4-10, n and o=0. Said NIS may be called NIS1a3 herein.

In one embodiment, detergent formulations comprise at least two non-ionic surfactants, both selected from compounds of the general formula (NIS1a), wherein one of said non-ionic surfactants is characterized in R¹ being n-C₉H₁₉, R² and R⁵ being H, m being 5-7, n and o=0, and the other surfactant is characterized in R¹ being n-C₁₇H₃₅, R² and R⁵ being H, m being 5-7, n and o=0. Said NIS may be called NIS1a4 herein.

In one embodiment, detergent formulations comprise at least two non-ionic surfactants, both selected from compounds of the general formula (NIS1a), wherein one of said non-ionic surfactants is characterized in R¹ being n-C₁₁H₂₃, R⁵ being H, m being 7, n and o=0, and the other surfactant is characterized in R¹ being C₁₃H₂₇, R⁵ being H, m being 7, n and o=0. Said NIS may be called NIS1a5 herein.

In one embodiment, detergent formulations comprise at least one non-ionic surfactant according to the general formula (NIS1a) with R¹ being C₃ to C₁₈ linear alkyl, R² being H, R³ and R⁴, each independently selected from
methyl with n or o being 2-25, or
ethyl and n or o being 1-3, or
propyl and n or o being 1-3,
   and wherein m+n+o equals 5-50. In one embodiment, R⁵ is H. In one embodiment, R⁵ is selected from methyl, butyl, benzyl and t-butyl. Said NIS may be called NIS1a6 herein.

The non-ionic surfactants of the general formulae (NIS1a) and (NIS1b) may be of any structure, is it block or random structure, and is not limited to the displayed sequence of formulae (NIS1a) and (NIS1b).

In one embodiment, detergent formulations comprise at least one compound according to the general formula (NIS1a) with R² being H, m being 10-50, R³ being linear or branched C₈-C₁₂ alkyl, n being 1 or 2 with 1 being preferred, o being 0 or 1 and R⁵ being H.

In one embodiment, detergent formulations comprise at least one compound according to general formula (NIS1a) with R² being H, m being 10-50, R³ being linear or branched C₈-C₁₂ alkyl, n being 1 or 2 with 1 being preferred, o being 0 or 1 and R⁵ being H.

In one embodiment, detergent formulations comprise at least one non-ionic surfactant selected from compounds according to formula (NIS1a) with R¹ being n-C₈ alkyl, R² being H, R³ being branched C₁₁ alkyl, R⁵ being H, m being 22, n being 1 and o being 0. Said NIS may be called NIS1a7 herein.

In one embodiment, detergent formulations comprise at least one non-ionic surfactant selected from compounds according to formula (NIS1a) with R¹ being n-C₈ alkyl, R² being H, R³ being branched C₁₁ alkyl, R⁵ being H, m being 19, n being 1 and o being 0. Said NIS may be called NIS1a8 herein.

In one embodiment, detergent formulations comprise at least one non-ionic selected from compounds according to formula (NIS1a) with R¹ being n-C₈ alkyl, R² being H, R³ being n-C₈-C₁₀ alkyl, R⁵ being H, m being 40, n being 1 and o being 0. Said NIS may be called NIS1a9 herein.

In one embodiment, detergent formulations comprise at least one non-ionic surfactant selected from compounds according to formula (NIS1a) with R¹ being n-C₈ alkyl, R² being H, R³ being methyl, R⁴ being n-C₁₀ alkyl, R⁵ being H, m being 22, n being 1 and o being 1. Said NIS may be called NIS1a10 herein.

### NIS2

In one embodiment, detergent formulations comprise at least one non-ionic surfactant selected from compounds of the general formula (NIS2), which might be called alkylpolyglycosides (APG) herein:

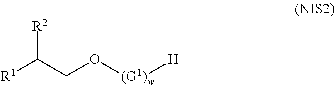

R¹ in general formula (NIS2) is selected from C₁-C₁₇ alkyl and C₂-C₁₇ alkenyl, wherein alkyl and/or alkenyl are linear (straight-chain; n-) or branched; examples are n-C₇H₁₅, n-C₉H₁₉, n-C₁₁H₂₃, n-C₁₃H₂₇, n-C₁₅H₃₁, n-C₁₇H₃₅, i-C₉H₁₉, i-C₁₂H₂₅.
R² in general formula (NIS2) is selected from H, C₁-C₁₇ alkyl and C₂-C₁₇ alkenyl, wherein alkyl and/or alkenyl are linear (straight-chain; n-) or branched.
G1 in general formula (NIS2) is selected from monosaccharides with 4 to 6 carbon atoms, such as glucose and xylose.

The integer w of the general formula (NIS2) is in the range of from 1.1 to 4, w being an average number.

### NIS3

In one embodiment, detergent formulations comprise at least one non-ionic surfactant selected from compounds of general formula (NIS3):

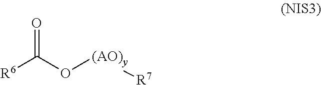

The variables of the general formula (NIS3) are defined as follows:
AO is selected from ethylene oxide (EO), propylene oxide (PO), butylene oxide (BO), and mixtures thereof.
R⁶ is selected from C₅-C₁₇ alkyl and C₅-C₁₇ alkenyl, wherein alkyl and/or alkenyl are linear (straight-chain; n-) or branched.
R⁷ is selected from H, C₁-C₁₈ alkyl, wherein alkyl is linear (straight-chain; n-) or branched.

The integer y of the general formula (NIS3) is a number in the range of 1 to 70, preferably 7 to 15.

### NIS4

In one embodiment, detergent formulations comprise at least one non-ionic surfactant selected from sorbitan esters (NIS4a) and/or ethoxylated (NIS4b) or propoxylated (NIS4c) sorbitan esters. Non-limiting examples are products sold under the trade names SPAN and TWEEN.

### NIS5

In one embodiment, detergent formulations comprise at least one non-ionic surfactant selected from alkoxylated mono- or di-alkylamines (NIS5a), fatty acid monoethanolamides (FAMA, NIS5b), fatty acid diethanolamides (FADA, NIS5c), ethoxylated fatty acid monoethanolamides (EFAM, NIS5d), propoxylated fatty acid monoethanolamides (PFAM, NIS5e), polyhydroxy alkyl fatty acid amides (NIS5f), or N-acyl N-alkyl derivatives of glucosamine (NIS5g) such as glucamides (GA), fatty acid glucamide (FAGA) and combinations thereof.

In one embodiment LDF of the invention comprise
(A) at least one serine protease,
(B) at least one alkanolamine formate,
(C) at least one anionic surfactant (C1) and/or at least one builder (C2), and
(D) at least one non-ionic surfactant (D1) selected from NIS1a2, NIS1a3, NIS1a4, NIS1a5, NIS2, NIS3, NIS4, and NIS5.

Preferably such LDF comprise C1 in amounts ranging from 10% to 30% by weight, preferably 12% to 25% by weight, relative to the total weight of the LDF. C2 is preferably comprised in amounts ranging from 1% to 5% by weight relative to the total weight of the LDF. D1 is preferably comprised in amounts ranging from 5% to 15% by weight relative to the total weight of the LDF.

In one embodiment, LDF1 to LDF261 comprise NIS1a2, NIS1a3, NIS1a4, NIS1a5, NIS2, NIS3, NIS4, or NIS5.

In one embodiment, LDF28 comprises at least one NPB1, preferably sodium citrate dihydrate, and NIS1a2, NIS1a3, NIS1a4, NIS1a5, NIS2, NIS3, NIS4, or NIS5.

In one embodiment, LDF58 comprises at least one NPB1, preferably sodium citrate dihydrate, and NIS1a2, NIS1a3, NIS1a4, NIS1a5, NIS2, NIS3, NIS4, or NIS5.

In one embodiment, LDF88 comprises at least one NPB1, preferably sodium citrate dihydrate, and NIS1a2, NIS1a3, NIS1a4, NIS1a5, NIS2, NIS3, NIS4, or NIS5.

In one embodiment, LDF28 comprises at least one NPB2, preferably Na₂CO₃, and NIS1a2, NIS1a3, NIS1a4, NIS1a5, NIS2, NIS3, NIS4, or NIS5.

In one embodiment, LDF58 comprises at least one NPB2, preferably Na₂CO₃, and NIS1a2, NIS1a3, NIS1a4, NIS1a5, NIS2, NIS3, NIS4, or NIS5.

In one embodiment, LDF88 comprises at least one NPB2, preferably Na₂CO₃, and NIS1a2, NIS1a3, NIS1a4, NIS1a5, NIS2, NIS3, NIS4, or NIS5.

In one embodiment, LDF28 comprises at least one NPB3, preferably selected from NPBAC1, NPBAC2 and NPBAC3, and NIS1a2, NIS1a3, NIS1a4, NIS1a5, NIS2, NIS3, NIS4, or NIS5.

In one embodiment, LDF58 comprises at least one NPB3, preferably selected from NPBAC1, NPBAC2 and NPBAC3, and NIS1a2, NIS1a3, NIS1a4, NIS1a5, NIS2, NIS3, NIS4, or NIS5.

In one embodiment, LDF88 comprises at least one NPB3, preferably selected from NPBAC1, NPBAC2 and NPBAC3.

In one embodiment, LDF88 comprises at least one NPB3, preferably selected from NPBAC1, NPBAC2 and NPBAC3, and NIS1a2, NIS1a3, NIS1a4, NIS1a5, NIS2, NIS3, NIS4, or NIS5.

In one embodiment LDF of the invention comprise
(A) at least one serine protease,
(B) at least one alkanolamine formate,
(C) at least one builder (C2), and
(D) at least one non-ionic surfactant (D1) selected from NIS1a1, NIS1a6, NIS1a7, NIS1a8, NIS1a9, and NIS1a10.

Preferably, such LDF are essentially devoid of C1. C2 is preferably comprised in amounts ranging from 5% to 20% by weight relative to the total weight of the LDF. D1 is preferably comprised in amounts below 5% by weight relative to the total weight of the LDF.

In one embodiment, LDF127 to LDF261 comprise NIS1a1, NIS1a6, NIS1a7, NIS1a8, NIS1a9 or NIS1a10, wherein LDF127 to LDF261 are essentially devoid of C1.

### Component D2—Further Hydrolase

In one embodiment LDF of the invention comprise
(A) 0.0005% to 0.4% by weight of at least one hydrolase (EC 3), preferably protease as described herein,
(B) 4% to 20% by weight of a compound according to formula (I) as described herein and
(C) at least one anionic compound, wherein the anionic compound is selected from a surface-active anionic compound (C1) and a complexing anionic compound (C2), and
(D) at least one hydrolase different from hydrolase EC 3, preferably wherein the hydrolase different from hydrolase EC 3 is selected from the group consisting of protease, amylase, lipase, cellulase, mannanase, as described herein, preferably wherein hydrolase EC 3 is a protease as described herein and the hydrolase different from hydrolase EC 3 is preferably an amylase, preferably at least one alpha-amylase (EC 3.2.1.1).

Preferably, LDF comprise at least one alpha-amylase selected from hybrid amylases. In one embodiment, LDF comprise at least one hybrid amylase, which is at least 95% identical to SEQ ID NO: 23 of WO 2014/183920 (Amy1). In one embodiment, LDF comprise at least one hybrid amylase, which is at least 95% identical to SEQ ID NO: 30 of WO 2014/183921 (Amy2). In one embodiment, LDF comprise at least one hybrid amylase, which is at least 95% identical to SEQ ID NO: 54 of WO 2021/032881 (Amy3).

In one embodiment, LDF1 to LDF126 comprise at least one hybrid amylase Amy1, Amy2 or Amy3 and optionally comprises NIS1a2, NIS1a3, NIS1a4, NIS1a5, NIS2, NIS3, NIS4, or NIS5 and optionally C2 as disclosed herein.

In one embodiment, LDF127 to LDF261 comprise at least one hybrid amylase Amy1, Amy2, or Amy3. Said LDF preferably are essentially devoid of C1. In one embodiment, said LDF comprise NIS1a1, NIS1a6, NIS1a7, NIS1a8, NIS1a9, or NIS1a10.

In one embodiment, LDF comprise at least one lipase, preferably at least one triacylglycerol lipase (EC 3.1.1.3) as described herein.

Preferably, LDF comprise at least one at least one triacylglycerol lipase, which is at least 80% identical to amino acids 1-269 of SEQ ID NO: 2 of U.S. Pat. No. 5,869,438. In one embodiment, said lipase comprises at least the amino acid substitutions T231R and N233R (Lip1). In one embodiment, LDF comprise at least one lipase comprising T231R and N233R and one or more of the following amino acid exchanges when compared to amino acids 1-269 of SEQ ID NO: 2 of U.S. Pat. No. 5,869,438: Q4V, V60S, A150G, L227G, P256K.

In one embodiment, LDF comprise at least one lipase at least 95% identical to the full-length polypeptide sequence of amino acids 1-269 of SEQ ID NO: 1 of WO 2015/010009, preferably comprising at least the amino acid substitutions N11K+A18K+G23K+K24A+V77I+D130A+V1541+V187T+T189Q (Lip2a) or N11K+A18K+G23K+K24A+L75R+V77I+D130A+V154|+V187T+T189Q (Lip2b).

In one embodiment, LDF1 to LDF126 comprise at least one triacylglycerol lipase Lip1 and optionally comprises NIS1a2, NIS1a3, NIS1a4, NIS1a5, NIS2, NIS3, NIS4, or NIS5 and optionally C2 as disclosed herein.

In one embodiment, LDF1 to LDF126 comprise at least one triacylglycerol lipase Lip2a and optionally comprises NIS1a2, NIS1a3, NIS1a4, NIS1a5, NIS2, NIS3, NIS4, or NIS5 and optionally C2 as disclosed herein.

In one embodiment, LDF comprise at least one cellulase (D2c), preferably at least one beta-1,4-glucanase (EC 3.2.1.4), also called endoglucanase herein.

In one embodiment, LDF comprise at least one Humicola insolens DSM 1800 endoglucanase at least 80% identical to the amino acid sequence disclosed in FIG. 14A-E of WO 91/17244, preferably to the sequence according to amino acids 20-434 (Cel1). Preferably said endoglucanase has one or more substitutions at positions selected from 182, 223, and 231, most preferably selected from P182S, A223V, and A231V. In one embodiment, LDF comprise at least one endoglucanase at least 80% identical to a polypeptide according to SEQ ID NO: 2 of WO 95/02675.

In one embodiment, LDF comprise at least one Bacillus sp. endoglucanase, which is at least 80% identical to the amino acid sequence of position 1 to position 773 of SEQ ID NO: 2 of WO 2004/053039.

In one embodiment, LDF comprise at least one Thielavia terrestris endoglucanase, which is at least 80% identical to the amino acid sequence of position 1 to position 299 of SEQ ID NO: 4 of WO 2004/053039.

In one embodiment, LDF1 to LDF126 comprise at least one endoglucanase Cel1 and optionally comprises NIS1a2, NIS1a3, NIS1a4, NIS1a5, NIS2, NIS3, NIS4, or NIS5 and optionally C2 as disclosed herein.

In one aspect, LDF comprise at least one mannanase, preferably at least one beta-mannanase (EC 3.2.1.78).

In one embodiment, LDF comprise at least one beta-mannanase selected from the GH5 mannanase family. In one embodiment, LDF comprise at least one beta-mannanase at least 90% identical to SEQ ID NO: 12 of WO 2018/184767 (Man1). In one embodiment, LDF comprise at least one beta-mannanase at least 90% identical to SEQ ID NO: 16 of WO 2018/184767 (Man2). In one embodiment, LDF comprise at least one beta-mannanase at least 90% identical to SEQ ID NO: 20 of WO 2018/184767 (Man3). Preferably, LDF comprise at least one mannanase 95% identical to a polypeptide sequence of SEQ ID NO: 20 of WO 2018/184767 having at least one substitution selected from A101V, E405G, and Y459F.

In one embodiment, LDF comprise at least one beta-mannanase which has a polypeptide sequence at least 90% identical to amino acids 29-324 of SEQ ID NO: 1 of WO 2021/058452 (Man4).

In one embodiment, LDF comprise at least one beta-mannanase originating from Trichoderma organisms, such as those disclosed in WO 93/24622. Preferably, at least one beta-mannanase is 80% identical to SEQ ID NO: 1 of WO 2008/009673 (Man5). More preferably, the beta-mannanase according to SEQ ID NO: 1 of WO 2008/009673 comprises at least one substitution selected from S3R, S66P, N113Y, V181H, L207F, A215T and F274L.

In one embodiment, LDF1 to LDF126 comprise at least one beta-mannanase Man1, Man2, Man3, Man4, or Man5 and optionally comprises NIS1a2, NIS1a3, NIS1a4, NIS1a5, NIS2, NIS3, NIS4, or NIS5 and optionally C2 as disclosed herein.

In one aspect, LDF1 to LDF126 comprise at least one DNAse.

In one embodiment, LDF comprise at least one DNAse at least 80% identical to SEQ ID NO: 1-24 and SEQ ID NO: 27-28 of WO 2019/081724 and WO 2019/081721. Preferably, LDF comprise at least one DNAse comprising one or both motifs selected from SEQ ID NO: 25 and SEQ ID NO: 26 of WO 2019/081724.

In one embodiment, LDF comprise at least one DNAse comprising one or more motifs selected from SEQ ID NO: 73, SEQ ID NO: 74 and SEQ ID NO: 75 of WO 2017/060493.

In one embodiment, LDF1 to LDF126 comprise at least one DNAse and optionally comprises NIS1a2, NIS1a3, NIS1a4, NIS1a5, NIS2, NIS3, NIS4, or NIS5 and optionally C2 as disclosed herein.

### Component D3—Solvent

In one embodiment LDF of the invention comprise
(A) 0.0005% to 0.4% by weight of at least one hydrolase (EC 3)
(B) 4% to 20% by weight of a compound according to formula (I) as described herein and
(C) at least one anionic surfactant (C1) and/or at least one builder (C2) and
(D) at least one solvent (D3) selected from water (SOL1) and organic solvents.

Preferably, at least one organic solvent is selected from water-miscible organic solvents.

“Water-miscibility” of an organic solvent means the property of the organic solvent to mix with water forming a homogeneous solution. Thus, a “solution” in this context means a homogeneous mixture of two or more organic solvents in water. When one of the solvents in a solution is water, the solution may be called “aqueous solution”. “Homogeneity” usually refers to uniform formulations of two or more components within a solution or mixture.

In one embodiment, LDF comprise at least one organic solvent selected from monohydric alcohols (SOL2), dihydric alcohols (SOL3), trihydric alcohols (SOL4) and sugar alcohols (SOL5).

At least one monohydric alcohol (SOL2) is selected from C₂H₆O, 1-propanol, propan-2-ol, 1-butanol, 2-methyl-1-propanol, butan-2-ol, ethylene glycol methyl ether, ethylene glycol ethyl ether, ethylene glycol propyl ether, and ethylene glycol phenyl ether.

Water-miscible SOL2 are preferably selected from C₂H₆O, and propan-2-ol.

At least one dihydric alcohol (SOL3) is selected from butane-1,3-diol, pentane-1,4-diol, pentane-1,5-diol, pentane-2,4-diol, hexane-2,5-diol, vicinal diols (OH-groups at vicinal C; SOL3a) and alpha-omega diols (OH-groups located at one of the ends of a linear molecule (HO—ROH), SOL3b).

In one embodiment, LDF comprise at least one vicinal diol (SOL3a) preferably selected from ethan-1,2-diol, propane-1,2-diol, butane-1,2-diol, butane-2,3-diol, pentane-1,2-diol, pentane-2,3-diol, hexane-2,3-diol, hexane-3,4-diol, heptane-1,2-diol, heptane-2,3-diol, heptane-3,4-diol, octane-1,2-diol, octane-2,3-diol, octane-3,4-diol, and octane-4,5-diol.

In one embodiment, LDF comprise at least one alpha-omega diol (SOL3b) preferably selected from, butane-1,4-diol, hexane-1,6-diol, propane-1,3-diol, 2-(2-hydroxyethoxy) ethanol, 2-(2-propoxyethoxy) ethanol, 2-(2-butoxyethoxy) ethanol and 2-methyl-2,4-pentandiol.

Water-miscible SOL3 are preferably selected from butane-1,3-diol, propane-1,2-diol, pentane-1,4-diol, pentane-1,5-diol, pentane-2,4-diol, butane-1,4-diol, and 1,6-hexane diol.

As a trihydric alcohol (SOL4) propane-1,2,3-triol may be comprised.

LDF, in one embodiment, comprise at least one sugar alcohol (alditol, SOL5) such as sorbitol, mannitol and erythriol, with sorbitol being preferred.

Further, LDF may comprise at least one organic solvent selected from compounds such as 2-butoxyethanol, isopropyl alcohol, and d-limonene.

In one embodiment, LDF1 to LDF126 comprise SOL2, preferably ethylene glycol phenyl ether.

In one embodiment, LDF1 to LDF126 comprise SOL3a, preferably propane-1,2-diol.

In one embodiment, LDF1 to LDF126 comprise SOL4, preferably propane-1,2,3-triol.

In one embodiment, LDF1 to LDF126 comprise SOL5, preferably sorbitol.

In one embodiment, said LDF1 to LDF126 comprising SOL2, SOL3a, SOL4, or SOL5 comprise NIS1a2 and optionally C2 as disclosed herein.

In one embodiment, said LDF1 to LDF126 comprising SOL2, SOL3a, SOL4, or SOL5 comprise NIS1a3 and optionally C2 as disclosed herein.

In one embodiment, said LDF1 to LDF126 comprising SOL2, SOL3a, SOL4, or SOL5 comprise NIS1a4 and optionally C2 as disclosed herein.

In one embodiment, said LDF1 to LDF126 comprising SOL2, SOL3a, SOL4, or SOL5 comprise NIS1a5 and optionally C2 as disclosed herein.

In one embodiment, said LDF1 to LDF126 comprising SOL2, SOL3a, SOL4, or SOL5 comprise NIS2 and optionally C2 as disclosed herein.

In one embodiment, said LDF1 to LDF126 comprising SOL2, SOL3a, SOL4, or SOL5 comprise NIS3 and optionally C2 as disclosed herein.

In one embodiment, said LDF1 to LDF126 comprising SOL2, SOL3a, SOL4, or SOL5 comprise NIS4 and optionally C2 as disclosed herein.

In one embodiment, said LDF1 to LDF126 comprising SOL2, SOL3a, SOL4, or SOL5 comprise NIS5 and optionally C2 as disclosed herein.

In one embodiment, said LDFs further comprise SOL1.

In one embodiment, LDF127 to LDF261 comprise SOL2, preferably ethylene glycol phenyl ether. Said LDF preferably are essentially devoid of C1.

In one embodiment, LDF127 to LDF261 comprise SOL3a, preferably propane-1,2-diol. Said LDF preferably are essentially devoid of C1.

In one embodiment, LDF127 to LDF261 comprise SOL4, preferably propane-1,2,3-triol. Said LDF preferably are essentially devoid of C1.

In one embodiment, LDF127 to LDF261 comprise SOL5, preferably sorbitol. Said LDF preferably are essentially devoid of C1.

In one embodiment, said LDF127 to LDF261 comprising SOL2, SOL3a, SOL4, or SOL5 comprise NIS1a1.

In one embodiment, said LDF127 to LDF261 comprising SOL2, SOL3a, SOL4, or SOL5 comprise NIS1a6.

In one embodiment, said LDF127 to LDF261 comprising SOL2, SOL3a, SOL4, or SOL5 comprise NIS1a7.

In one embodiment, said LDF127 to LDF261 comprising SOL2, SOL3a, SOL4, or SOL5 comprise NIS1a8.

In one embodiment, said LDF127 to LDF261 comprising SOL2, SOL3a, SOL4, or SOL5 comprise NIS1a9.

In one embodiment, said LDF127 to LDF261 comprising SOL2, SOL3a, SOL4, or SOL5 comprise NIS1a10.

In one embodiment, said LDFs further comprise SOL1.

### Component D4—Polymers

In one embodiment LDF of the invention comprise
(A) 0.0005% to 0.4% by weight of at least one hydrolase (EC 3),
(B) 4% to 20% by weight of a compound according to formula (I) as described herein,
(C) at least one anionic surfactant (C1) and/or at least one builder (C2), and
(D) at least one polymer (D4).

At least one polymer, in one embodiment is selected from the group of “polycarboxylates”, which include salts of polycarboxylates. Salt forming cations may be monovalent or multivalent. Suitable examples include but are not limited to sodium, potassium, magnesium, calcium, ammonium, and the ammonium salt of mono-, di- and triethanolamine.

Polycarboxylates include compounds comprising monomers selected from unsaturated carboxylic acids of the general formula (CoC):

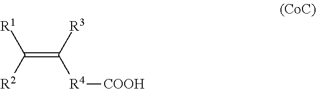

The variables in general formula (CoC) are defined as follows:
R¹, R² and R³ are independently selected from H; linear or branched C₁-C₁₂ alkyl, linear or branched C₂-C₁₂ alkenyl, wherein alkyl and/or alkenyl may be substituted with —NH₂, —OH, or COOH; —COOH; and —COOR⁵, wherein R⁵ is selected from linear or branched C₁-C₁₂ alkyl and linear or branched C₂-C₁₂ alkenyl.
R⁴ may be a spacer group, which is optionally selected from —(CH₂)ₙ— with n being in the range of 0 to 4, —COO—(CH₂)ₖ— with k being in the range of 1 to 6, —C(O)—NH— and —C(O)—NR⁶—, wherein R⁶ is selected from linear or branched C₁-C₂₂ alkyl, linear or branched C₂-C₂₂ alkenyl, and C₆-C₂₂ aryl.

Non-limiting examples of suitable unsaturated carboxylic acids include acrylic acid, methacrylic acid, 2-ethylacrylic acid, 2-phenylacrylic acid, malonic acid, crotonic acid, maleic acid (or maleic anhydride), fumaric acid, itaconic acid, aconitic acid, mesaconic acid, citraconic acid, sorbic acid, cinnamic acid, methylenemalonic acid, unsaturated C₄-C₁₀ dicarboxylic acids, and mixtures thereof.

Polycarboxylates may be characterized by having a K-value, i.e. the molecular weight determined according to Fikentscher's K-value, which is a value measured via the viscosity of the aqueous solution at a defined polymer content and defined viscosity measurement conditions and thus correlates to the molecular weight of the polymer for a given polymer class. The measurement is preferably done according to ISO 1628-1.

Polycarboxylates may be characterized by their weight average molecular mass (Mw). Preferably Mw is determined by gel permeation chromatography using standard methodology. Examples include Polycarboxylates having weight average molecular weights (Mw) in the range of about 500 g/mol to about 500,000 g/mol, in the range of about 1,000 g/mol to about 100,000 g/mol, or in the range of about 3,000 g/mol to about 80,000 g/mol.

Polycarboxylates may be homopolymers with the repeating monomer being the same unsaturated carboxylic acid according to formula CoC. Such homopolymers may be called “HP” herein.

In one embodiment, LDF comprise at least one homopolymer of acrylic acid, which may be called “HP1” herein. HP1 herein include salts of polyacrylic acid. Salt forming cations may be monovalent or multivalent including sodium, potassium, magnesium, calcium, ammonium, and the ammonium salt of mono-, di- and triethanolamine, with sodium being preferred. HP1 may thereby be fully or only partially neutralized by salt formation.

In one embodiment, LDF comprise at least one HP1, preferably sodium salt of polyacrylic acid, having a weight average molecular mass (Mw; measured with gel permeation chromatography) in the range of about 800 g/mol to about 12,000 g/mol, preferably in the range of about 900 g/mol to about 10,000 g/moly, more preferably in the range of about 1,000 g/mol to about 9,000 g/mol. At least one CoHP1 may have a mean molar mass selected from about 1,200 g/mol (HP1a), about 2,500 g/mol (HP1b), about 4,000 g/mol (HP1c) and about 8,500 g/mol (HP1d).

In one embodiment, LDF comprise at least one HP1, preferably sodium salt of polyacrylic acid, having a K-value in the range of about 10 to about 50, preferably in the range of about 15 to about 30, wherein the K-value is determined with about 1% dry substance in distilled water. At least one HP1 may have a K-value selected from about 15 (HP1a), about 20 (HP1b), about 25 (HP1c) and about 30 (HP1d).

In one embodiment, LDF comprise at least one HP1. Specifically, one of the following combinations may be comprised:

| id | actual combinations |
|---|---|
| LDF262 | EPr9iA + AAF1a + HP1a |
| LDF263 | EPr9iA + AAF1a + HP1b |
| LDF264 | EPr9iA + AAF1a + HP1c |
| LDF265 | EPr9iA + AAF1a + HP1d |
| LDF266 | EPr9iA + AAF2a + HP1a |
| LDF267 | EPr9iA + AAF2a + HP1b |
| LDF268 | EPr9iA + AAF2a + HP1c |
| LDF269 | EPr9iA + AAF2a + HP1d |
| LDF270 | EPr9iA + AAF3a + HP1a |
| LDF271 | EPr9iA + AAF3a + HP1b |
| LDF272 | EPr9iA + AAF3a + HP1c |
| LDF273 | EPr9iA + AAF3a + HP1d |
| LDF274 | LDF262 + AS1c |
| LDF275 | LDF262 + AS2b |
| LDF276 | LDF262 + AS4 |
| LDF279 | LDF262 + AS1c + AS2b + AS4 |
| LDF280 | LDF263 + AS1c |
| LDF281 | LDF263 + AS2b |
| LDF282 | LDF263 + AS4 |
| LDF283 | LDF263 + AS1c + AS2b + AS4 |
| LDF284 | LDF264 + AS1c |
| LDF285 | LDF264 + AS2b |
| LDF286 | LDF264 + AS4 |
| LDF287 | LDF264 + AS1c + AS2b + AS4 |
| LDF288 | LDF265 + AS1c |
| LDF289 | LDF265 + AS2b |
| LDF290 | LDF265 + AS4 |
| LDF291 | LDF265 + AS1c + AS2b + AS4 |
| LDF292 | LDF266 + AS1c |
| LDF293 | LDF266 + AS2b |
| LDF294 | LDF266 + AS4 |
| LDF295 | LDF266 + AS1c + AS2b + AS4 |
| LDF296 | LDF267 + AS1c |
| LDF297 | LDF267 + AS2b |
| LDF298 | LDF267 + AS4 |
| LDF299 | LDF267 + AS1c + AS2b + AS4 |
| LDF300 | LDF268 + AS1c |
| LDF301 | LDF268 + AS2b |
| LDF302 | LDF268 + AS4 |
| LDF303 | LDF268 + AS1c + AS2b + AS4 |
| LDF304 | LDF269 + AS1c |
| LDF305 | LDF269 + AS2b |
| LDF306 | LDF269 + AS4 |
| LDF307 | LDF269 + AS1c + AS2b + AS4 |
| LDF308 | LDF270 + AS1c |
| LDF309 | LDF270 + AS2b |
| LDF310 | LDF270 + AS4 |
| LDF311 | LDF270 + AS1c + AS2b + AS4 |
| LDF312 | LDF271 + AS1c |
| LDF313 | LDF271 + AS2b |
| LDF314 | LDF271 + AS4 |
| LDF315 | LDF271 + AS1c + AS2b + AS4 |
| LDF316 | LDF272 + AS1c |
| LDF317 | LDF272 + AS2b |
| LDF318 | LDF272 + AS4 |
| LDF319 | LDF272 + AS1c + AS2b + AS4 |
| LDF320 | LDF273 + AS1c |
| LDF321 | LDF273 + AS2b |
| LDF322 | LDF273 + AS4 |
| LDF323 | LDF273 + AS1c + AS2b + AS4 |
| LDF324 | LDF311 + NPB1 |
| LDF325 | LDF311 + NPB2 |
| LDF326 | LDF311 + NPBAC1 |
| LDF327 | LDF311 + NPBAC2 |
| LDF328 | LDF311 + NPBAC3 |
| LDF329 | LDF315 + NPB1 |
| LDF330 | LDF315 + NPB2 |
| LDF331 | LDF315 + NPBAC1 |
| LDF332 | LDF315 + NPBAC2 |
| LDF333 | LDF315 + NPBAC3 |
| LDF334 | LDF319 + NPB1 |
| LDF335 | LDF319 + NPB2 |
| LDF336 | LDF319 + NPBAC1 |
| LDF337 | LDF319 + NPBAC2 |
| LDF338 | LDF319 + NPBAC3 |
| LDF339 | LDF323 + NPB1 |
| LDF340 | LDF323 + NPB2 |
| LDF341 | LDF323 + NPBAC1 |
| LDF342 | LDF323 + NPBAC2 |
| LDF343 | LDF323 + NPBAC3 |

In one embodiment, EPr9iA in LDF262 to LDF343 is EPr9iA having R101E.

In one embodiment, LDF262 to LDF343 further comprise PSB1, PBS2, Amy1, Amy2, Amy3, Cel1, Lip1, or Lip2a.

In one embodiment, LDF262 to LDF343 further comprise Man1 or Man2 or Man3 or Man4 or Man5, preferably Man4.

In one embodiment, LDF262 to LDF343 further comprise at least one NIS selected from NIS1a2, NIS1a3, NIS1a4, NIS1a5, NIS2, NIS3, NIS4 and NIS 5.

In one embodiment, LDF comprise one of the following combinations:

| id | actual combinations |
|---|---|
| LDF344 | LDF262 + NPB1 |
| LDF345 | LDF262 + NPB2 |
| LDF346 | LDF262 + NPBAC1 |
| LDF347 | LDF262 + NPBAC2 |
| LDF348 | LDF262 + NPBAC3 |
| LDF349 | LDF263 + NPB1 |
| LDF350 | LDF263 + NPB2 |
| LDF351 | LDF263 + NPBAC1 |
| LDF352 | LDF263 + NPBAC2 |
| LDF353 | LDF263 + NPBAC3 |
| LDF354 | LDF264 + NPB1 |
| LDF356 | LDF264 + NPB2 |
| LDF357 | LDF264 + NPBAC1 |
| LDF358 | LDF264 + NPBAC2 |
| LDF359 | LDF264 + NPBAC3 |
| LDF360 | LDF265 + NPB1 |
| LDF361 | LDF265 + NPB2 |
| LDF362 | LDF265 + NPBAC1 |
| LDF363 | LDF265 + NPBAC2 |
| LDF364 | LDF265 + NPBAC3 |
| LDF365 | LDF266 + NPB1 |
| LDF366 | LDF266 + NPB2 |
| LDF367 | LDF266 + NPBAC1 |
| LDF368 | LDF266 + NPBAC2 |
| LDF369 | LDF266 + NPBAC3 |
| LDF370 | LDF267 + NPB1 |
| LDF371 | LDF267 + NPB2 |
| LDF372 | LDF267 + NPBAC1 |
| LDF373 | LDF267 + NPBAC2 |
| LDF374 | LDF267 + NPBAC3 |
| LDF375 | LDF268 + NPB1 |
| LDF376 | LDF268 + NPB2 |
| LDF377 | LDF268 + NPBAC1 |
| LDF378 | LDF268 + NPBAC2 |
| LDF379 | LDF268 + NPBAC3 |
| LDF380 | LDF269 + NPB1 |
| LDF381 | LDF269 + NPB2 |
| LDF382 | LDF269 + NPBAC1 |
| LDF383 | LDF269 + NPBAC2 |
| LDF384 | LDF269 + NPBAC3 |
| LDF385 | LDF270 + NPB1 |
| LDF386 | LDF270 + NPB2 |
| LDF387 | LDF270 + NPBAC1 |
| LDF388 | LDF270 + NPBAC2 |
| LDF389 | LDF270 + NPBAC3 |
| LDF390 | LDF271 + NPB1 |
| LDF391 | LDF271 + NPB2 |
| LDF392 | LDF271 + NPBAC1 |
| LDF393 | LDF271 + NPBAC2 |
| LDF394 | LDF271 + NPBAC3 |
| LDF395 | LDF272 + NPB1 |
| LDF396 | LDF272 + NPB2 |
| LDF397 | LDF272 + NPBAC1 |
| LDF398 | LDF272 + NPBAC2 |
| LDF399 | LDF272 + NPBAC3 |
| LDF400 | LDF273 + NPB1 |
| LDF401 | LDF273 + NPB2 |
| LDF402 | LDF273 + NPBAC1 |
| LDF403 | LDF273 + NPBAC2 |
| LDF404 | LDF273 + NPBAC3 |
| LDF405 | LDF270 + C-NPB2 |
| LDF406 | LDF270 + C-NPB3 |
| LDF407 | LDF270 + C-NPB4 |
| LDF408 | LDF270 + C-NPB5 |
| LDF409 | LDF270 + C-NPB6 |
| LDF410 | LDF270 + C-NPB7 |
| LDF411 | LDF270 + C-NPB8 |
| LDF412 | LDF271 + C-NPB2 |
| LDF413 | LDF271 + C-NPB3 |
| LDF414 | LDF271 + C-NPB4 |
| LDF415 | LDF271 + C-NPB5 |
| LDF416 | LDF271 + C-NPB6 |
| LDF417 | LDF271 + C-NPB7 |
| LDF418 | LDF271 + C-NPB8 |
| LDF419 | LDF272 + C-NPB2 |
| LDF420 | LDF272 + C-NPB3 |
| LDF421 | LDF272 + C-NPB4 |
| LDF422 | LDF272 + C-NPB5 |
| LDF423 | LDF272 + C-NPB6 |
| LDF424 | LDF272 + C-NPB7 |
| LDF425 | LDF272 + C-NPB8 |
| LDF426 | LDF273 + C-NPB2 |
| LDF427 | LDF273 + C-NPB3 |
| LDF428 | LDF273 + C-NPB4 |
| LDF429 | LDF273 + C-NPB5 |
| LDF430 | LDF273 + C-NPB6 |
| LDF431 | LDF273 + C-NPB7 |
| LDF432 | LDF273 + C-NPB8 |

In one embodiment, LDF344 to LDF432 are essentially devoid of anionic surfactants.

In one embodiment, EPr9iA in LDF344 to LDF432 is EPr9iA having R101E.

In one embodiment, LDF344 to LDF432 further comprise PSB1, PSP2, Amy1, Amy2, or Amy3.

In one embodiment, LDF344 to LDF432 further comprise at least one NIS selected from NIS1a1, NIS1a6, NIS1a7, NIS1a8, NIS 1a9 and NIS1a10.

In one embodiment, LDF comprise at least one copolymer of acrylic acid and maleic acid, which may be called “CP1” herein. CP1 herein include salts of copolymer of acrylic acid and maleic acid. Salt forming cations may be monovalent or multivalent including sodium, potassium, magnesium, calcium, ammonium, and the ammonium salt of mono-, di- and triethanolamine, with sodium being preferred. CP1 may thereby be fully or only partially neutralized by salt formation.

In one embodiment, CP1 comprise 50% to 90% by weight acrylic acid and 50% to 10% by weight maleic acid.

In one embodiment, LDF comprise at least one CP1, preferably sodium salt of the copolymer of acrylic acid and maleic acid, having a weight average molecular mass (Mw; measured with gel permeation chromatography) in the range of about 30,000 g/mol to about 100,000 g/mol, preferably in the range of about 50,000 g/mol to about 90,000 g/mol, more preferably in the range of about 70,000 g/mol to about 85,000 g/mol. At least one CP1 may have a mean molar mass of about 70,000 g/mol.

In one embodiment, LDF comprise at least one CP1, preferably sodium salt of the copolymer of acrylic acid and maleic acid, having a K-value in the range of about 10 to about 100, preferably in the range of about 30 to about 80, more preferably in the range of about 45 to about 60, wherein the K-value is determined with about 1% dry substance in distilled water. At least one CP1 may have a K-value of about 55.

In one embodiment, LDF comprise at least one copolymer consisting of acrylic acid and at least one hydrophobic monomer, which may be called “CP2a” herein. Suitable hydrophobic monomers are, for example, isobutene, diisobutene, butene, pentene, hexene and styrene, olefins with 10 or more carbon atoms or mixtures thereof, such as, for example, 1-decene, 1-dodecene, 1-tetradecene, 1-hexadecene, 1-octadecene, 1-eicosene, 1-docosene, 1-tetracosene and 1-hexacosene, C₂₂-α-olefin, a mixture of C₂₀-C₂₄-α-olefins and polyisobutene having on average 12 to 100 carbon atoms per molecule. Such copolymers may be called CoCP2 herein and include partially or completely neutralized forms thereof. Neutralization is preferably achieved by using suitable bases such as NaOH or KOH to form the alkali metal salts of such polymer.

In one embodiment, LDF comprise at least one copolymer consisting of maleic acid and at least one hydrophobic monomer as disclosed above. The hydrophobic monomer preferably is selected from the group consisting of isobutene, diisobutene, butene, or mixtures thereof.

Neutralization is preferably achieved by using suitable bases such as NaOH or KOH to form the alkali metal salts of such a polymer.

In one embodiment, the copolymer comprises maleic acid and a hydrophobic monomer, preferably diisobutene, in a ratio of 1:1, which may be called “CP2b” herein. Preferably, said CP2b is the sodium salt of the copolymer of maleic acid and diisobutene. CP2b may have a K-value in the range of about 20 to about 80, preferably in the range of about 0 to about 50, more preferably in the range of about 35 to about 45, wherein the K-value is determined with about 1% dry substance in distilled water.

In one embodiment, LDF comprise at least one copolymer consisting of at least one monomer according to formula CoC and at least one monomer with at least one sulfonate group.

Said copolymers may be called “CP3” herein and preferably consists of acrylic acid and AMPS. CP3 herein include salts of said copolymers. Salt forming cations may be monovalent or multivalent including sodium, potassium, magnesium, calcium, ammonium, and the ammonium salt of mono-, di- and triethanolamine, with sodium being preferred. CP3 may thereby be fully or only partially neutralized by salt formation.

In one embodiment, CP3 comprise about 60% to 80% by weight acrylic acid and 20% to 40% by weight AMPS. Preferably, the weight ratio acrylic acid:AMPS, is about 70:30. CP3 may have a K-value of about 40.

In one embodiment, LDF comprise at least one copolymer comprising at least one monomer from the group consisting of unsaturated carboxylic acids as defined in formula CoC with at least one hydrophilic monomer selected from non-ionic monomers with hydroxyl function or alkylene oxide groups. Such copolymers may be called “CP4” herein and include salts of said polymers. Salt forming cations may be monovalent or multivalent including sodium, potassium, magnesium, calcium, ammonium, and the ammonium salt of mono-, di- and triethanolamine, with sodium being preferred. CP4 may thereby be fully or only partially neutralized by salt formation.

Examples of such non-ionic monomers with hydroxyl function or alkylene oxide groups include but are not limited to allyl alcohol, isoprenol, methoxypolyethylene glycol (meth)acrylate, methoxypolypropylene glycol (meth)acrylate, methoxypolybutylene glycol (meth)acrylate, methoxypoly(propylene oxide-co-ethylene oxide) (meth)acrylate, ethoxypolyethylene glycol (meth)acrylate, ethoxypolypropylene glycol (meth)acrylate, ethoxypolybutylene glycol (meth)acrylate and ethoxypoly(propylene oxide-co-ethylene oxide) (meth)acrylate. Polyalkylene glycols here may comprise 3 alkylene oxide units (AO) to 50 AO per molecule, 5 AO to 40 AO per molecule, or 10 AO to 30 AO per molecule.

In one embodiment, LDF comprise at least one polycarboxylates, which is derivatized by alkoxylation such as ethoxylation and/or propoxylation. Such polycarboxylates may be called “CP5” herein.

Alkoxylated polycarboxylates comprise polyacrylates having one ethoxy sidechain per every 2 to 8 acrylate units. In one embodiment alkoxylated polycarboxylates comprise polyacrylates having one ethoxy sidechain per every 7 to 8 acrylate units. The sidechains are ester-linked to the polyacrylate “backbone” to provide a “comb” polymer type structure. The molecular weight may be in the range of about 2,000 g/mol to about 50,000 g/mol.

In one embodiment, LDF comprise at least one amphoteric polymer comprising
(a) at least one ethylenically unsaturated carboxylic acid selected from acrylic acid and methacrylic acid,
(b) at least one amide, selected from N—C₁-C₁₀-alkyl(meth)acrylamide, acrylamide and methacrylamide, and
(c) at least one comonomer selected from DADMAC (poly-diallyl dimethylammonium chlorid), MAPTAC (3-methacrylamido-N,N,N-trimethylpropan-1-ammonium chloride) and APTAC ((3-acrylamidopropyl)-trimethylammonium chloride).

Such amphoteric polymers may be called “CP6” herein.

In one embodiment, LDF comprise one or more compounds selected from the group of polyaspartic acids and their salts, which may be called “CP7” herein. Salt forming cations may be monovalent or multivalent. Suitable examples include but are not limited to sodium, potassium, magnesium, calcium, ammonium, and the ammonium salt of mono-, di- and triethanolamine.

CP7 may be prepared from aspartic acid, maleic acid or maleic anhydride or fumaric acid, which either are reacted to obtain first polysuccinimide (which in turn may be hydrolyzed to polyaspartic acid or directly to the salts of polyaspartic acid) or directly polyaspartic acid or directly the salts thereof. Preferred is the use of L-aspartic acid as the sole starting material for the preparation of the thermal polyaspartic acid.

In one aspect, CP7 includes compounds, which are produced by polycondensation of aspartic acid and other carboxylic acids such as amino acids. In another aspect, CP7 includes compounds, which are produced by polycondensation of aspartic acid, diamines and aminoalcohols. CP7 thus includes copolymers of aspartic acid and their salts.

In one embodiment, LDF comprise one or more compounds selected from the group of alkoxylated polyalkylene imine or alkoxylated polyamines, which may be called “CP8” herein. “CP8” also includes the structures disclosed in WO 2021/165468, in particular in claim 1 and on pages 2 to 4 of WO 2021/165468 and the structures obtained by processes described in WO 2022/136408 and in WO 2022/136409, in particular in claim 1 and on page 3 of WO 2022/136408 and WO 2022/136409, respectively. The structures of the alkoxylated polyalkylene imine or alkoxylated polyamine may be further described by the general formula (CP8a)

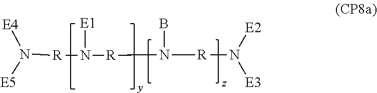

in which the variables are each defined as follows:
R represents identical or different,
   i) linear or branched C₂-C₁₂-alkylene radicals or
   ii) an etheralkyl unit of the following formula (CP8b):

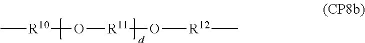

in which the variables are each defined as follows:
R¹⁰, R¹¹, R¹² represent identical or different, linear or branched C₂-C₆-alkylene radicals and
d is an integer having a value in the range of 0 to 50 or
iii) C₅-C₁₀-cycloalkylene radicals optionally substituted with at least one C₁-C₃-alkyl;
B represents a continuation of the alkoxylated polyalkylene imine by branching
y and z are each an integer having a value in the range of 0 to 150, under the proviso that both z and y are 0 in case R are C₅-C₁₀-cycloalkylene radicals optionally substituted with at least one C₁-C₃-alkyl
E1, E2, E3, E4, E5 hydrogen or represent an identical or different residue according to formula (CP8c),
   wherein the residue according to formula (CP8c) is an alkylenoxy unit defined as follows

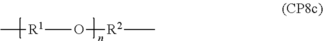

in which the variables are each defined as follows:
R¹ represents C₂-C₂₂-(1,2-alkylene) radicals;
R² represents hydrogen and/or C₁-C₂₂-alkyl and/or C₇-C₂₂-aralkyl in case z is an integer≥1 within general formula (CP8b), or
R² represents hydrogen and/or C₁-C₄-alkyl and/or C₇-C₂₂-aralkyl in case z is 0 within general formula (CP8b);
n is an integer having a value of at least 5 to 100;
   wherein 20 to 100% of the total amount of E1, E2, E3, E4 and E5 in general formula (CP8a) is a residue according to formula (CP8c).

In one embodiment, the nitrogen atoms present in CP8 are quaternized, in order to adjust the alkoxylated polyalkylene imines or the alkoxylated polyamines to the particular formulation to achieve better compatibility and/or phase stability of the formulation.

LDF may comprise CP8 in an amount ranging from 0.1% to 10% by weight, preferably from about 0.25% to 5% by weight, more preferably from about 0.5% to 3% by weight, and most preferably from about 1% to 3% by weight, all % by weight relative to the total weight of the detergent formulation.

In one embodiment. LDF comprise at least one of the following combinations:

| id | actual combinations |
|---|---|
| LDF433 | EPr9iA + AAF3a + CP1 |
| LDF434 | EPr9iA + AAF3a + CP2a |
| LDF435 | EPr9iA + AAF3a + CP2b |
| LDF436 | EPr9iA + AAF3a + CP3 |
| LDF437 | EPr9iA + AAF3a + CP4a |
| LDF438 | EPr9iA + AAF3a + CP5 |
| LDF439 | EPr9iA + AAF3a + CP6 |
| LDF440 | EPr9iA + AAF3a + CP7 |
| LDF441 | EPr9iA + AAF3a + CP8 |
| LDF442 | LDF433 + AS1c |
| LDF443 | LDF433 + AS2b |
| LDF444 | LDF433 + AS4 |
| LDF445 | LDF433 + AS1c + AS2b + AS4 |
| LDF446 | LDF434 + AS1c |
| LDF447 | LDF434 + AS2b |
| LDF448 | LDF434 + AS4 |
| LDF449 | LDF434 + AS1c + AS2b + AS4 |
| LDF450 | LDF435 + AS1c |
| LDF451 | LDF435 + AS2b |
| LDF452 | LDF435 + AS4 |
| LDF453 | LDF435 + AS1c + AS2b + AS4 |
| LDF454 | LDF436 + AS1c |
| LDF455 | LDF436 + AS2b |
| LDF456 | LDF436 + AS4 |
| LDF457 | LDF436 + AS1c + AS2b + AS4 |
| LDF458 | LDF437 + AS1c |
| LDF459 | LDF437 + AS2b |
| LDF460 | LDF437 + AS4 |
| LDF461 | LDF437 + AS1c + AS2b + AS4 |
| LDF462 | LDF438 + AS1c |
| LDF463 | LDF438 + AS2b |
| LDF464 | LDF438 + AS4 |
| LDF465 | LDF438 + AS1c + AS2b + AS4 |
| LDF466 | LDF439 + AS1c |
| LDF467 | LDF439 + AS2b |
| LDF468 | LDF439 + AS4 |
| LDF469 | LDF439 + AS1c + AS2b + AS4 |
| LDF470 | LDF440 + AS1c |
| LDF471 | LDF440 + AS2b |
| LDF472 | LDF440 + AS4 |
| LDF473 | LDF440 + AS1c + AS2b + AS4 |
| LDF474 | LDF441 + AS1c |
| LDF475 | LDF441 + AS2b |
| LDF476 | LDF441 + AS4 |
| LDF477 | LDF441 + AS1c + AS2b + AS4 |
| LDF478 | LDF465 + NPB1 |
| LDF479 | LDF465 + NPB2 |
| LDF480 | LDF465 + NPBAC1 |
| LDF481 | LDF465 + NPBAC2 |
| LDF482 | LDF465 + NPBAC3 |
| LDF483 | LDF469 + NPB1 |
| LDF484 | LDF469 + NPB2 |
| LDF485 | LDF469 + NPBAC1 |
| LDF486 | LDF469 + NPBAC2 |
| LDF487 | LDF469 + NPBAC3 |
| LDF488 | LDF473 + NPB1 |
| LDF489 | LDF473 + NPB2 |
| LDF490 | LDF473 + NPBAC1 |
| LDF491 | LDF473 + NPBAC2 |
| LDF492 | LDF473 + NPBAC3 |
| LDF493 | LDF477 + NPB1 |
| LDF494 | LDF477 + NPB2 |
| LDF495 | LDF477 + NPBAC1 |
| LDF496 | LDF477 + NPBAC2 |
| LDF497 | (E) LDF477 + NPBAC3 |

In one embodiment, EPr9iA in LDF433 to LDF497 is EPr9iA having R101E.

In one embodiment, LDF433 to LDF497 further comprise PSB1, PSP2, Amy1, Amy2, Amy3, Cel1, Lip1, or Lip2a.

In one embodiment, LDF433 to LDF497 further comprise Man1 or Man2 or Man3 or Man4 or Man5, preferably Man4.

In one embodiment, LDF433 to LDF497 further comprise at least one NIS selected from NIS1a2, NIS1a3, NIS1a4, NIS1a5, NIS2, NIS3, NIS4 and NIS 5.

In one embodiment, LDF comprise one of the following combinations:

| id | actual combinations |
|---|---|
| LDF498 | LDF433 + NPB1 |
| LDF499 | LDF433 + NPB2 |
| LDF500 | LDF433 + NPBAC1 |
| LDF501 | LDF433 + NPBAC2 |
| LDF502 | LDF433 + NPBAC3 |
| LDF503 | LDF434 + NPB1 |
| LDF504 | LDF434 + NPB2 |
| LDF505 | LDF434 + NPBAC1 |
| LDF506 | LDF434 + NPBAC2 |
| LDF507 | LDF434 + NPBAC3 |
| LDF508 | LDF435 + NPB1 |
| LDF509 | LDF435 + NPB2 |
| LDF510 | LDF435 + NPBAC1 |
| LDF511 | LDF435 + NPBAC2 |
| LDF512 | LDF435 + NPBAC3 |
| LDF513 | LDF436 + NPB1 |
| LDF514 | LDF436 + NPB2 |
| LDF515 | LDF436 + NPBAC1 |
| LDF516 | LDF436 + NPBAC2 |
| LDF517 | LDF436 + NPBAC3 |
| LDF518 | LDF436 + NPB1 |
| LDF519 | LDF436 + NPB2 |
| LDF520 | LDF436 + NPBAC1 |
| LDF521 | LDF436 + NPBAC2 |
| LDF522 | LDF436 + NPBAC3 |
| LDF523 | LDF437 + NPB1 |
| LDF524 | LDF437 + NPB2 |
| LDF525 | LDF437 + NPBAC1 |
| LDF526 | LDF437 + NPBAC2 |
| LDF527 | LDF437 + NPBAC3 |
| LDF528 | LDF437 + NPB1 |
| LDF529 | LDF437 + NPB2 |
| LDF530 | LDF437 + NPBAC1 |
| LDF531 | LDF437 + NPBAC2 |
| LDF532 | LDF437 + NPBAC3 |
| LDF533 | LDF438 + NPB1 |
| LDF534 | LDF438 + NPB2 |
| LDF535 | LDF438 + NPBAC1 |
| LDF536 | LDF438 + NPBAC2 |
| LDF537 | LDF438 + NPBAC3 |
| LDF538 | LDF439 + NPB1 |
| LDF539 | LDF439 + NPB2 |
| LDF540 | LDF439 + NPBAC1 |
| LDF541 | LDF439 + NPBAC2 |
| LDF542 | LDF439 + NPBAC3 |
| LDF543 | LDF440 + NPB1 |
| LDF544 | LDF440 + NPB2 |
| LDF545 | LDF440 + NPBAC1 |
| LDF546 | LDF440 + NPBAC2 |
| LDF547 | LDF440 + NPBAC3 |
| LDF548 | LDF441 + NPB1 |
| LDF549 | LDF441 + NPB2 |
| LDF550 | LDF441 + NPBAC1 |
| LDF551 | LDF441 + NPBAC2 |
| LDF552 | LDF441 + NPBAC3 |
| LDF553 | LDF433 + C-NPB2 |
| LDF554 | LDF433 + C-NPB3 |
| LDF555 | LDF433 + C-NPB4 |
| LDF556 | LDF433 + C-NPB5 |
| LDF557 | LDF433 + C-NPB6 |
| LDF558 | LDF433 + C-NPB7 |
| LDF559 | LDF433 + C-NPB8 |
| LDF560 | LDF434 + C-NPB2 |
| LDF561 | LDF434 + C-NPB3 |
| LDF562 | LDF434 + C-NPB4 |
| LDF563 | LDF434 + C-NPB5 |
| LDF564 | LDF434 + C-NPB6 |
| LDF565 | LDF434 + C-NPB7 |
| LDF566 | LDF434 + C-NPB8 |
| LDF567 | LDF435 + C-NPB2 |
| LDF568 | LDF435 + C-NPB3 |
| LDF569 | LDF435 + C-NPB4 |
| LDF570 | LDF435 + C-NPB5 |
| LDF571 | LDF435 + C-NPB6 |
| LDF572 | LDF435 + C-NPB7 |
| LDF573 | LDF435 + C-NPB8 |
| LDF574 | LDF436 + C-NPB2 |
| LDF575 | LDF436 + C-NPB3 |
| LDF576 | LDF436 + C-NPB4 |
| LDF577 | LDF436 + C-NPB5 |
| LDF578 | LDF436 + C-NPB6 |
| LDF579 | LDF436 + C-NPB7 |
| LDF580 | LDF436 + C-NPB8 |
| LDF581 | LDF437 + C-NPB2 |
| LDF582 | LDF437 + C-NPB3 |
| LDF583 | LDF437 + C-NPB4 |
| LDF584 | LDF437 + C-NPB5 |
| LDF585 | LDF437 + C-NPB6 |
| LDF586 | LDF437 + C-NPB7 |
| LDF587 | LDF437 + C-NPB8 |
| LDF588 | LDF438 + C-NPB2 |
| LDF589 | LDF438 + C-NPB3 |
| LDF590 | LDF438 + C-NPB4 |
| LDF591 | LDF438 + C-NPB5 |
| LDF592 | LDF438 + C-NPB6 |
| LDF593 | LDF438 + C-NPB7 |
| LDF594 | LDF438 + C-NPB8 |
| LDF595 | LDF439 + C-NPB2 |
| LDF596 | LDF439 + C-NPB3 |
| LDF597 | LDF439 + C-NPB4 |
| LDF598 | LDF439 + C-NPB5 |
| LDF599 | LDF439 + C-NPB6 |
| LDF600 | LDF439 + C-NPB7 |
| LDF601 | LDF439 + C-NPB8 |
| LDF602 | LDF440 + C-NPB2 |
| LDF603 | LDF440 + C-NPB3 |
| LDF604 | LDF440 + C-NPB4 |
| LDF605 | LDF440 + C-NPB5 |
| LDF606 | LDF440 + C-NPB6 |
| LDF607 | LDF440 + C-NPB7 |
| LDF608 | LDF440 + C-NPB8 |

In one embodiment, LDF498 to LDF608 are essentially devoid of anionic surfactants.

In one embodiment, EPr9iA in LDF498 to LDF608 is R101E.

In one embodiment, LDF498 to LDF608 further comprise PSB1.

In one embodiment, LDF498 to LDF608 further comprise PSP2.

In one embodiment, LDF498 to LDF608 further comprise Amy1.

In one embodiment, LDF498 to LDF608 further comprise Amy2.

In one embodiment, LDF498 to LDF608 further comprise Amy3.

In one embodiment, LDF498 to LDF608 further comprise at least one NIS selected from NIS1a1, NIS1a6, NIS1a7, NIS1a8, NIS 1a9 and NIS1a10.

### Component D5—Antimicrobial

In one embodiment LDF of the invention comprise
(A) 0.0005% to 0.4% by weight of at least one hydrolase (EC 3),
(B) 4% to 20% by weight of a compound according to formula (I) as described herein,
(C) at least one anionic surfactant (C1) and/or at least one builder (C2), and
(D) at least one antimicrobial (D5).

In one embodiment, LDF of the invention comprise at least one D5 selected from 2-phenoxyethanol (AMic1; CAS-No. 122-99-6), 2-bromo-2-nitropropane-1,3-diol (AMic2), formic acid in acid form or as its salt (AMic3), 4,4′-dichloro 2-hydroxydiphenylether (AMic4; CAS-No. 3380-30-1), and an isothiazol-3-one (AMic5).

In one embodiment, LDF1 to LDF608 comprise AMic1 in amounts ranging from 0.1% to 2% by weight relative to the total weight of the liquid formulation.

In one embodiment, LDF1 to LDF608 comprise AMic2 in amounts ranging from 20 ppm to 1000 ppm.

In one embodiment, LDF1 to LDF608 comprise AMic3 in amounts ranging from 0.05% to 0.5% by weight relative to the total weight of the liquid formulation.

In one embodiment, LDF1 to LDF608 comprise AMic4 in amounts ranging from 0.001% to 3% by weight, 0.002% to 1% by weight, or 0.01% to 0.6% by weight, all relative to the total weight of the liquid formulations.

### Component D6—Other Ingredients

LDF disclosed herein, in one embodiment further comprise at least one amphoteric surfactant selected from AMS1, AMS2, AMS3 and AMS4.

In one embodiment, detergent formulations comprise at least one amphoteric surfactant selected from compounds of the general formula (AMS1), which might be called modified amino acids (proteinogenic as well as non-proteinogenic):

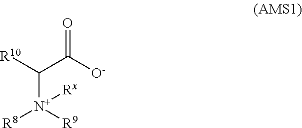

The variables in general formula (AMS1) are defined as follows:
R⁸ is selected from H, C₁-C₄ alkyl, C₂-C₄ alkenyl, wherein alkyl and/or are linear (straight-chain; n-) or branched.
R⁹ is selected from C₁-C₂₂ alkyl, C₂-C₂₂ alkenyl, C₁₀-C₂₂ alkylcarbonyl, and C₁₀-C₂₂ alkenylcarbonyl.
R¹⁰ is selected from H, methyl, —(CH₂)₃NHC(NH)NH₂, —CH₂C(O)NH₂, —CH₂C(O)OH, —(CH₂)₂C(O)NH₂, —(CH₂)₂C(O)OH, (imidazole-4-yl)-methyl, —CH(CH₃) C₂H₅, —CH₂CH(CH₃)₂, —(CH₂)₄NH₂, benzyl, hydroxymethyl, —CH(OH)CH₃, (indole-3-yl)-methyl, (4-hydroxy-phenyl)methyl, isopropyl, —(CH₂)₂SCH₃, and —CH₂SH.
R^{x} is selected from H and C₁-C₄-alkyl.

### AMS2

In one embodiment, detergent formulations comprise at least one amphoteric surfactant selected from compounds of general formulae (AMS2a), (AMS2b), or (AMS2c), which might be called betaines and/or sulfobetaines (AMS2):

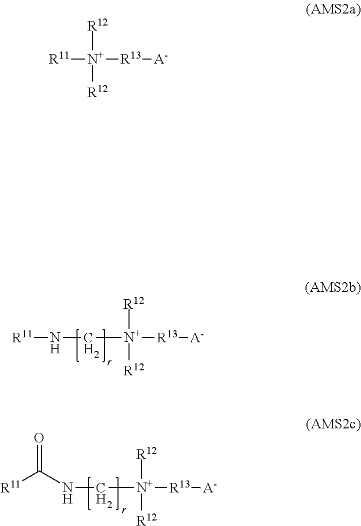

The variables in general formulae (AMS2a), (AMS2b) and (AMS2c) are defined as follows:
R¹¹ is selected from linear (straight-chain; n-) or branched C₇-C₂₂ alkyl and linear (straight-chain; n-) or branched C₇-C₂₂ alkenyl.
R¹² are each independently selected from linear (straight-chain; n-) C₁-C₄ alkyl.
R¹³ is selected from C₁-C₅ alkyl and hydroxy C₁-C₅ alkyl, for example, 2-hydroxypropyl.
A⁻ is selected from carboxylate and sulfonate.

The integer r in general formulae (AMS2a), (AMS2b), and (AMS2c) is in the range of 2 to 6.

### AMS3

In one embodiment, detergent formulations comprise at least one amphoteric surfactant selected from compounds of general formula (AMS3), which might be called alkylamphocarboxylates:

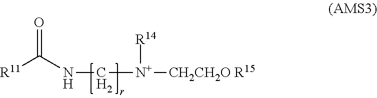

The variables in general formula (AMS3) are defined as follows:
R¹¹ is selected from C₇-C₂₂ alkyl and C₇-C₂₂ alkenyl, wherein alkyl and/or alkenyl are linear (straight-chain; n-) or branched, preferably linear.
R¹⁴ is selected from —CH₂C(O)O⁻M⁺, —CH₂CH₂C(O)O⁻M+ and —CH₂CH(OH)CH₂SO₃⁻M⁺.
R¹⁵ is selected from H and —CH₂C(O)O⁻

The integer r in general formula (AMS3) is in the range of 2 to 6.

M+ is selected from salt forming cations. Salt forming cations may be monovalent or multivalent; hence M+ equals 1/v Mv+. Examples include but are not limited to sodium, potassium, magnesium, calcium, ammonium, and the ammonium salt of mono-, di, and triethanolamine.

### AMS4

In one embodiment, detergent formulations comprise at least one amphoteric surfactant selected from compounds of general formula (AMS4), which might be called amine oxides (AO):

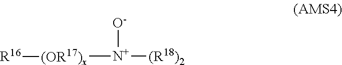

The variables in general formula (AMS4) are defined as follows:
R¹⁶ is selected from C₈-C₁₈ alkyl, hydroxy C₈-C₁₈ alkyl, acylamidopropoyl and C₈-C₁₈ alkyl phenyl group; wherein alkyl and/or alkenyl are linear (straight-chain; n-) or branched
R¹⁷ is selected from C₂-C₃ alkylene, hydroxy C₂-C₃ alkylene, and mixtures thereof
R¹⁸ is selected from C₁-C₃ alkyl and hydroxy C₁-C₃

The integer x in general formula (AMS4) is in the range of 0 to 5, preferably from 0 to 3, most preferably 0.

LDF comprising anionic surfactants (C1), in one embodiment comprise at least one graft polymer “GP1”, comprising as a graft base a polyether and as grafted side chains copolymers comprising at least one comonomer selected from

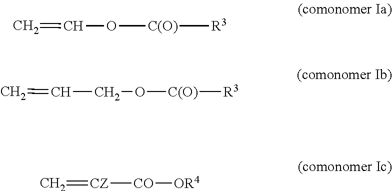

wherein R³ is selected from C₁-C₂₁ alkyl, for example methyl, n-propyl, n-pentyl, n-heptyl, n-nonyl, iso-nonyl, n-undecyl, n-tridecyl, n-pentadecyl, n-heptadecyl, or n-nonadecyl.
R⁴ is selected from C₂-C₂₀ alkyl, preferably with an even number of carbon atoms, for example ethyl, n- and iso propyl, n-hexyl, isohexyl, sec-hexyl, n-heptyl, n-octyl, 2-ethylhexyl, n-nonyl, n-decyl or isodecyl, n-C₁₂H₂₅, n-C₁₄H₂₉, n-C₁₆H₃₃ or n-C₁₈H₃₇, and Z is selected from hydrogen and methyl, hydrogen bring preferred.

In one embodiment, polyethers are polyethylene glycols, for example with an average molecular weight Mₙ in the range of from 500 to 25,000 g/mol, preferably 1,000 to 15,000 g/mol and even more preferably 1,500 to 10,000 g/mol.

In one embodiment, polyethers are polypropylene glycols, for example with an average molecular weight Mₙ in the range of from 500 to 20,000 g/mol, preferably 2,000 to 10,000 g/mol and even more preferably 4,000 to 9,000 g/mol.

In one embodiment, polyethers are copolymers of ethylene glycol and propylene glycol units, for example random copolymers and preferably block copolymers, for example di-block copolymers and tri-block copolymers.

In one embodiment, the graft base of GP1 is selected from polyethylene glycols, polypropylene glycols and EO-PO block copolymers, each non-capped or end-capped with C₁-C₂₀ alkyl or C₃-C₂₀-2-hydroxyalkyl. Polyethers are preferably non-capped.

Comonomer Ia specifically may be selected from vinylacetate, vinylpropionate, vinylbutyrate, vinyl-n-hexanoate, vinyl-n-octanoate, vinyl-2-ethylhexanoate, vinyllaurate, vinylstearate, vinylmyristate, and vinylpalmitate.

Comonomer Ib specifically may be selected from allylpropionate, allylbutyrate, allyl-n-hexanoate, allyl-n-octanoate, allyl-2-ethylhexanoate, allyllaurate, vinylstearate, allylmyristate, and allylpalmitate.

Comonomer Ic specifically may be selected from 2-ethylhexyl (meth)acrylate, 2-n-propylheptyl(meth)acrylate, stearyl(meth)acrylate, lauryl(meth)acrylate, palmityl(meth)acrylate, and myristyl(meth)acrylate.

In one embodiment, GP1 comprise side chains in copolymerized form preferably selected from comonomers of general formula (comonomer Ia) and (comonomer Ic).

In one embodiment, GP1 have an average molecular weight Mₙ in the range of from 2,250 to 200,000 g/mol, preferred are 2,250 to 25,000 g/mol, even more preferred are 2,500 to 10,000 g/mol. The average molecular weight Mₙ may be determined by gel permeation chromatography, with polyethylene glycol as comparison standard. The grafting as such may be confirmed by HPLC (High Pressure Liquid Chromatography).

LDF essentially devoid of anionic surfactants (C1), in one embodiment, comprise 0.05% to 0.4% by weight relative to the total weight of the detergent formulation of at least one compound selected from the group of non-alkoxylated polyalkylene imines (PEI).

In one embodiment, LDF comprise a branched homopolymer of 15-20 ethylenimine units. The homopolymer preferably has a Mw of about 250 g/mol to 1,500 g/mol. The ratio of primary:secondary:tertiary amine preferably is approximately 1:0.9:x when measured with 13C-NMR spectroscopy, wherein x is 0.5-0.6. Such homopolymers may be called PEI1 herein.

In one embodiment, LDF comprise a branched homopolymer having a Mw of about 500 g/mol to 10,000 g/mol. The ratio of primary:secondary:tertiary amine is preferably approximately 1:1:x when measured with 13C-NMR spectroscopy, wherein x is 0.7-0.9. Such homopolymers may be called PEI2 herein.

In one embodiment, LDF comprise a branched homopolymer having a Mw of about 25,000 g/mol and the ratio of primary:secondary:tertiary amine is approximately 1:1.1:0.7 when measured with 13C-NMR spectroscopy. Said homopolymer may be called PEI3 herein.

In one embodiment, LDF comprise a branched homopolymer having a Mw of about 70,000 g/mol and the ratio of primary:secondary:tertiary amine is approximately 0.5:1:0.5 when measured with 13C-NMR spectroscopy. Said homopolymer may be called PEI4 herein.

In one embodiment, LDF comprise a branched homopolymer having a Mw of about 750,000 g/mol and the ratio of primary:secondary:tertiary amine is approximately 1:1:0.7 when measured with 13C-NMR spectroscopy. Said homopolymer may be called PEI5 herein.

In one embodiment, LDF essentially devoid of anionic surfactants (C1) comprise 0.05% to 0.4% by weight relative to the total weight of the detergent formulation of at least one Zinc salt (ZS) selected from water-soluble and water-insoluble zinc salts. In this context, water-insoluble means zinc salts which, in distilled water at 25° C., have a solubility of 0.1 g/l or less. Thus, Zinc salts having a higher solubility in water are accordingly referred to water-soluble Zinc salts.

The Zinc salt may be selected from Zinc benzoate, Zinc gluconate, Zinc lactate, Zinc formate, ZnCl2, ZnSO4, Zinc acetate, Zinc citrate, Zn(NO3)2, Zn(CH3SO3)2 and Zinc gallate, with ZnCl2 (ZS1), ZnSO4 (ZS2), Zinc acetate (ZS3), Zinc citrate (ZS4), Zn(NO3)2 (ZS5), Zn(CH3SO3)2 (ZS6) and Zinc gallate (ZS7) being preferred.

In one embodiment, Zinc salt is selected from ZnO (ZS8), ZnO·aq (ZS9), Zn(OH)2 (ZS10) and ZnCO3 (ZS11). Preference is given to ZnO·aq.

In one embodiment, LDF essentially devoid of anionic surfactants (C₁) comprise at least one graft polymer “GP2” which is composed of
GP2-A: at least one graft base, which is selected from nonionic monosaccharides, disaccharides, oligosaccharides and polysaccharides, and side chains obtainable by grafting on of
GP2-B: at least one ethylenically unsaturated mono- or dicarboxylic acid, called monocarboxylic acid or dicarboxylic acid for short, and
GP2-C: at least one compound selected from

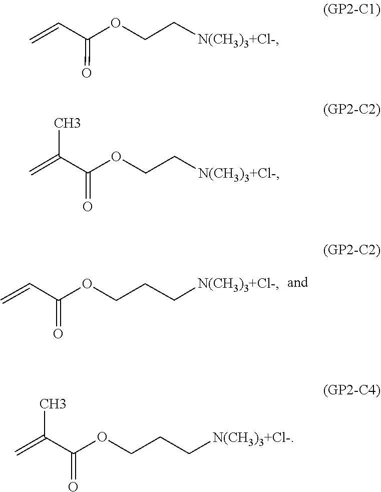

GP2-A is preferably selected from non-ionic polysaccharides, in particular from starch, which is preferably not chemically modified, for example hydroxyl groups thereof are preferably neither esterified nor etherified. Examples are corn starch, rice starch, potato starch, and wheat starch.
GP2-B is preferably selected from monocarboxylic acids, more preferably from ethylenically unsaturated C₃-C₁₀-monocarboxylic acids and the alkali metal or ammonium salts thereof, in particular the potassium and the sodium salts. Preferred monocarboxylic acids are acrylic acid and methacrylic acid, and also sodium (meth)acrylate. Mixtures of ethylenically unsaturated C₃-C₁₀ monocarboxylic acids and in particular mixtures of acrylic acid and methacrylic acid are also preferred.

### EXAMPLES

### Example 1: Liquid Enzyme Preparation with and without Primary Stabilizer (% Means % by Weight Relative to Total Weight of the LEP)

TABLE 1
Composition of the liquid enzyme preparation with
and without primary stabilizer (% means % by
weight relative to total weight of the LEP)

| LEP-I | LEP-IICo | LEP-II |
|---|---|---|
| enzyme concentrate | enzyme concentrate | enzyme concentrate |
| 0.3% primary enzyme | — | — |
| stabilizer | | |
| 0.2% CaCl₂*2H₂O | 0.2% CaCl₂*2H₂O | 0.2% CaCl₂*2H₂O |
| 50% 1,2-propane | 50% 1,2-propane | 50% triethanolamine |
| diol | diol | formate |

H₂O up to 100%
enzyme concentrate: EPr9iA having R101E; amount used in LEP adjusted to provide 6% protease with 100% proteolytic activity
primary enzyme stabilizer: Z-VAL

TABLE 2
Stability of protease determined by residual
enzymatic activity after storage

| | residual protease activity* | | | | |
|---|---|---|---|---|---|
| Storage time | 1 week | 4 weeks | 6 weeks | 8 weeks | 10 weeks |
| LEP-I | 98 | 96 | 90 | 87 | 78 |
| LEP-IICo | 72 | 49 | 33 | 24 | 12 |
| LEP-II | 98 | 97 | 93 | 88 | 82 |

*when compared to initial protease activity at t = 0, which has been set 100%.

The liquid enzyme preparations were stored at a temperature of 38° C. for up to 10 weeks. A 4-week storage is said to correspond to a storage of approximately 9 months at room temperature or >15 month at 8° C. The protease activity was analyzed by measuring the reactivity towards the peptidic substrate Suc-AAPF-pNA. Here pNA is cleaved from the substrate molecule at 30° C., pH 8.6 using 100 mM TRIS buffer. The rate of cleavage, directly proportional to the protease activity, can be determined by the increase of the yellow color of free pNA in the solution by measuring OD405, the optical density at 405 nm. Proteolytic activity was determined before and after storage.

### Example 2: Liquid Detergent Formulations Tested (% Means % by Weight Relative to the Total Weight of the Detergent Formulation) for Protease and Amylase

TABLE 3
Composition of the LDF tested for protease and amylase

| | Det 1 | Det 2 |
|---|---|---|
| Solid content prior to | about 35% | about 45% |
| addition of inventive | | |
| compounds (non H₂O) | | |
| LAS | 6.5% | 6.0% |
| AES | 6.5% | 16.0% |
| Coco fatty acid soap | 2.4% | 3.0% |
| Sodium citrate | 2.0% | 2.0% |
| NIS1 | 6.5% | — |
| NIS2 | — | — |
| NIS3 | — | 8.0% |
| alkanolamine | 4.0% MEA | 4.0% TEA |
| Propylene glycol | 5.0% | 5.0% |
| Ethanol | 3.0% | |
| Enzymes | 1% protease product | 1% protease product |
| | 0.5% amylase product | 0.5% amylase product |
| Component (b) | as indicated in the results Table 4 below | |
| ad H₂O | Up to 100% | |
| pH | 8.5 | 8.0 |

LAS: anionic surfactant; linear dodecylbenzene sulfonic acid (CAS 27176-87-0): Maranil DBS/LC
AES (alkyl ether sulphates); anionic surfactant; poly(oxy-1,2-ethanediyl), .alpha.-sulfo-.omega.-hydroxy-, C₁₂₋₁₄-alkyl ethers, sodium salts (CAS 68891-38-3): Texapon N70
NIS1: non-ionic surfactant; C₁₃C₁₅ Oxo Alcohol Ethoxylate (7 EO): Lutensol AO 7
NIS2: non-ionic surfactant; C₁₂C₁₄ Oxo Alcohol Ethoxylate (7 EO): Lutensol A 7N
NIS3: non-ionic surfactant; C₁₂C₁₈ Oxo Alcohol Ethoxylate (7 EO): Dehydol LT 7
MEA: monoethanolamine
TEA: triethanolamine
protease product: product comprising about 4% EPr9iA having R101E (having 100% proteolytic activity) in about 50% diol blend
amylase product: Amplify ® prime 100L (Novozymes)

TABLE 4
Storage stability of protease and amylase determined by residual
activity of said enzymes after certain time of storage at
38° C. in detergent formulations Det 1 and Det 2

| | | | | | residual | | |
|---|---|---|---|---|---|---|---|
| | residual protease | | | | amylase | | |
| | activity | | | | activity | | |
| Storage time in weeks | 1 | 2 | 4 | 6 | 1 | 4 | 6 |
| Det 1 without | 43 | 14 | 0 | 0 | 95 | 78 | 60 |
| component (b) | | | | | | | |
| Det 1 + 14% AAF1a | 75 | 52 | 42 | 29 | | | |
| Det 1 + 6% AAF3a | 76 | 63 | 50 | 35 | 96 | 91 | 86 |
| Det 1 + 16% AAF3a | 90 | 76 | 64 | 46 | 97 | 93 | 91 |
| Det 2 without | 36 | 10 | 0 | 0 | 94 | 80 | 64 |
| component (b) | | | | | | | |
| Det 2 + 4% AAF1a | 60 | 42 | 33 | 23 | | | |
| Det 2 + 6% AAF3a | 77 | 53 | 43 | 33 | 96 | 89 | 85 |
| Det 2 + 8% AAF3a | 88 | 77 | 62 | 52 | 96 | 92 | 90 |

The liquid detergent formulations were stored at a temperature of 38° C. for up to 6 weeks. A 4-week storage is said to correspond to a storage of approximately 9 months at room temperature or >15 month at 8° C.

The protease activity was analyzed by measuring the reactivity towards the peptidic substrate Suc-AAPF-pNA. Here pNA is cleaved from the substrate molecule at 30° C., pH 8.6 using 100 mM TRIS buffer. The rate of cleavage, directly proportional to the protease activity, can be determined by the increase of the yellow color of free pNA in the solution by measuring OD₄₀₅, the optical density at 405 nm. Proteolytic activity was determined before and after storage.

The amylase activity after storage was measured quantitatively by the release of the chromophore para-nitrophenol (pNP) from the substrate Ethyliden-blocked-pNPG7 (Roche Applied Science, material number 10880078103). The alpha-amylase degrades the substrate into smaller molecules and α-glucosidase (Roche Applied Science, material number 11626329103), which is added in excess compared to the α-amylase, process these smaller products until pNP is released; the release of pNP, measured via an increase of absorption at 405 nm, is directly proportional to the α-amylase activity of the sample. Amylase standard: Termamyl 120 L (Sigma 3403). Amylolytic activity was determined before and after storage.

Residual enzyme activity corresponds to the enzyme activity remaining when compared to the initial enzyme activity available before storage at time 0.

### Example 3: Liquid Detergent Formulations Tested with Protease, Amylase and Lipase

TABLE 5
Composition of liquid detergent formulations tested (% means % by weight relative
to the total weight of the detergent formulation) for protease, amylase and lipase

| | Det 1 | Det 2 | Det 3 | Det 4 | Det 5 |
|---|---|---|---|---|---|
| LAS | 6.5% | 3% | 6% | 12% | 20% |
| AES | 6.5% | 14% | 16% | 18% | 4% |
| NIS1a-I | 6.5% | — | — | — | 20% |
| NIS1a-II | — | 6.5% | — | — | — |
| NIS1a-III | — | — | 8% | 5% | 5% |
| Coco fatty acid soap | 2.4% | 2% | 3% | 5% | — |
| NaCitrate | 2% | 3% | 2% | 3% | 5% |
| Alkanolamine | 4% MEA | 4% MEA | 4% TEA | — | — |
| Propylene glycol | 5% | 7% | 5% | 7% | 10% |
| Ethanol | 3% | 3% | — | 5% | 5% |
| Enzymes* | 1% protease I | 1% protease I | 1% protease II | 1% protease II | 1% protease I |
| | 0.5% amylase | 0.5% amylase | 0.5% amylase | 0.5% amylase | 0.5% amylase |
| | 0.2% lipase | 0.2% lipase | 0.2% lipase | 0.2% lipase | 0.2% lipase |
| Component (b) | as indicated in the results tables below | | | | |
| add H2O | to 100% | | | | |
| pH | 8.5 | 8 | 8 | 8 | 8 |

LAS: anionic surfactant; linear dodecylbenzene sulfonic acid (CAS 27176-87-0): Maranil DBS/LC
AES (alkyl ether sulphates); anionic surfactant; poly(oxy-1,2-ethanediyl), .alpha.-sulfo-.omega.-hydroxy-, C₁₂₋₁₄-alkyl ethers, sodium salts (CAS 68891-38-3): Texapon N70
NIS1a-I: non-ionic surfactant; C₁₃C₁₅ Oxo Alcohol Ethoxylate (7 EO): Lutensol AO 7
NIS1a-II: non-ionic surfactant; C₁₂C₁₄ Oxo Alcohol Ethoxylate (7 EO): Lutensol A 7N
NIS1a-II: non-ionic surfactant; C₁₂C₁₈ Oxo Alcohol Ethoxylate (7 EO): Dehydol LT 7
MEA: monoethanolamine;
TEA: triethanolamine
*Enzymes: % relates to % by weight of enzyme product formulated into the detergent formulation, relative to the total weight of the detergent formulation
Protease I: enzyme formulation with 4% EPr9iA having R101E (having 100% proteolytic activity) + about 0.3% Z-VAL
Protease II: enzyme formulation with 4% EPr9iA having R101E (having 100% proteolytic activity) lacking enzyme stabilizer such as boron-containing compound and peptide stabilizer
Amylase: Amplify ® prime 100L (Novozymes)
Lipase: Lipolase ® 100L (CAS-No. 9001-62-1, EC-No. 232-619-9) purchased from Sigma-Aldrich.

TABLE 6
Stability of detergent formulations comprising an additive

| Additive: | Det 1 | Det 2 | Det 3 | Det 4 | Det 5 |
|---|---|---|---|---|---|
| 2% Na formate | clear | clear | turbid | turbid | turbid |
| 3% Na formate | turbid | turbid | turbid, | turbid, | turbid, |
| | | | phase | phase | phase |
| | | | separation | separation | separatior |
| 2%-10% AAF1a | clear | clear | clear | clear | clear |
| 2%-10% AAF2a | clear | clear | clear | clear | clear |
| 2%-10% AAF3a | clear | clear | clear | clear | clear |
| 2%-15% AAF3b | clear | clear | clear | clear | clear |
| 2%-12% AAF3c | clear | clear | clear | clear | clear |

TABLE 7
Residual activity of protease, amylase,
and lipase after storage at 38° C.

| | | | | | | | Residual | |
|---|---|---|---|---|---|---|---|---|
| | Residual protease | | | Residual amylase | | | lipase | |
| | activity | | | activity | | | activity | |
| Storage time (weeks) | 2 | 3 | 8 | 2 | 3 | 8 | 3 | 8 |
| Det 1 | 15 | 3 | 0 | 94 | 80 | 58 | 72 | 46 |
| Det 1 + 2% Na | 41 | 29 | 10 | | | | | |
| formate | | | | | | | | |
| Det 1 + 2% AAF1a | 37 | 26 | 11 | | | | | |
| Det 1 + 4% AAF1a | 44 | 32 | 13 | | | | | |
| Det 1 + 6% AAF1a | 48 | 35 | 24 | | | | | |
| Det 1 + 8% AAF1a | 54 | 39 | 28 | | | | | |
| Det 1 + 4% Na | 48 | 33 | 21 | | | | | |
| formate* | | | | | | | | |
| Det 1 + 4% AAF2a | 42 | 30 | 13 | | | | | |
| Det 1 + 8% AAF2a | 51 | 35 | 23 | | | | | |
| Det 1 + 6% AAF3a | 71 | 58 | 42 | 91 | 89 | 88 | | |
| Det 1 + 8% AAF3a | 81 | 72 | 52 | 92 | 90 | 90 | 75 | 47 |
| Det 1 + 8% AAF3b | 75 | 64 | 50 | 91 | 88 | 85 | | |
| Det 2 | 10 | 0 | 0 | 94 | 76 | 58 | 70 | 44 |
| Det 2 + 3% AAF1a | 43 | 31 | 22 | | | | | |
| Det 2 + 6% AAF3a | 73 | 57 | 45 | 91 | 90 | 88 | 75 | 49 |
| Det 2 + 8% AAF3a | 83 | 74 | 53 | 92 | 90 | 90 | 73 | 47 |
| Det 2 + 12% AAF3a | 88 | 83 | 66 | 92 | 90 | 90 | 75 | 51 |
| Det 3 | 17 | 5 | 0 | 94 | 77 | 55 | 72 | 49 |
| Det 3 + 3% Na | 47 | 35 | 24 | | | | | |
| formate | | | | | | | | |
| Det 3 + 3% AAF1a | 45 | 32 | 23 | | | | | |
| Det 3 + 6% AAF1a | 50 | 39 | 30 | 92 | 79 | 60 | | |
| Det 3 + 6% AAF3a | 60 | 46 | 39 | 93 | 91 | 90 | 74 | 56 |
| Det 3 + 10% AAF3a | 82 | 70 | 58 | 92 | 92 | 89 | 74 | 58 |
| Det 3 + 8% AAF3a + | 80 | 66 | 53 | 92 | 92 | 89 | 72 | 56 |
| 2% Na formate | | | | | | | | |
| Det 4 | 22 | 14 | 0 | 94 | 75 | 52 | 72 | 50 |
| Det 4 + 3% Na | 52 | 39 | 30 | | | | | |
| formate* | | | | | | | | |
| Det 4 + 8% AAF1a | 69 | 50 | 42 | | | | | |
| Det 4 + 6% AAF3a | 73 | 58 | 43 | 92 | 89 | 87 | 77 | 56 |
| Det 4 + 10% AAF3a | 90 | 76 | 69 | 92 | 90 | 90 | 76 | 58 |
| Det 5 | 25 | 18 | 5 | 94 | 75 | 51 | 72 | 50 |
| Det 5 + 3% Na | 55 | 41 | 34 | | | | | |
| formate* | | | | | | | | |
| Det 5 + 8% AAF1a | 72 | 50 | 41 | | | | | |
| Det 5 + 8% AAF3a | 79 | 62 | 51 | 92 | 90 | 89 | 76 | 58 |
| Det 5 + 14% AAF3a | 90 | 76 | 69 | 92 | 92 | 90 | 80 | 68 |

*Formulation turbid, not stable

Detergent formulations Det 1 and Det 3 as described above except the enzyme component, which was as follows:

| | Det 1b | Det 3b |
|---|---|---|
| Enzymes | 1% protease II | 1% protease II |
| | 0.5% Amylase | 0.5% Amylase |

TABLE 8
Residual activity or protease and amylase after storage at 38° C.

| | | | | | Residual | | |
|---|---|---|---|---|---|---|---|
| | Residual protease | | | | amylase | | |
| | activity | | | | activity | | |
| Storage time (weeks) | 1 | 2 | 4 | 6 | 1 | 4 | 6 |
| Det 1b | 43 | 14 | 0 | 0 | 95 | 78 | 60 |
| Det 1b + 14% AAF1a | 75 | 52 | 42 | 29 | | | |
| Det 1b + 6% AAF3a | 76 | 63 | 50 | 35 | 96 | 91 | 86 |
| Det 1b + 16% AAF3a | 90 | 76 | 64 | 46 | 97 | 93 | 91 |
| Det 3b | 36 | 10 | 0 | 0 | 94 | 80 | 64 |
| Det 3b + 4% AAF1a | 60 | 42 | 33 | 23 | | | |
| Det 3b + 6% AAF3a | 77 | 53 | 43 | 33 | 96 | 89 | 85 |
| Det 3b + 8% AAF3a | 88 | 77 | 62 | 52 | 96 | 92 | 90 |

The liquid detergent formulations were stored at a temperature of 38° C. for up to 6 or 8 weeks, respectively. A 4-week storage is said to correspond to a storage of approximately 9 months at room temperature or >15 month at 8° C.

The protease activity was analyzed by measuring the reactivity towards the peptidic substrate Suc-AAPF-pNA. Here pNA is cleaved from the substrate molecule at 30° C., pH 8.6 using 100 mM TRIS buffer. The rate of cleavage, directly proportional to the protease activity, can be determined by the increase of the yellow color of free pNA in the solution by measuring OD₄₀₅, the optical density at 405 nm. Proteolytic activity was determined before and after storage.

The amylase activity after storage was measured quantitatively by the release of the chromophore para-nitrophenol (pNP) from the substrate Ethyliden-blocked-pNPG7 (Roche Applied Science, material number 10880078103). The alpha-amylase degrades the substrate into smaller molecules and α-glucosidase (Roche Applied Science, material number 11626329103), which is added in excess compared to the α-amylase, process these smaller products until pNP is released; the release of pNP, measured via an increase of absorption at 405 nm, is directly proportional to the α-amylase activity of the sample. Amylase standard: Termamyl 120 L (Sigma 3403). Amylolytic activity was determined before and after storage.

Lipase activity was determined by employing pNitrophenol-valerate (2.4 mM pNP-C₅ in 100 mM Tris pH 8.0, 0.01% Triton X100) as a substrate. The absorption at 405 nm was measured at 20° C. every 30 seconds over 5 minutes. The slope (absorbance increase at 405 nm per minute) of the time dependent absorption-curve is directly proportional to the activity of the lipase. Residual enzyme activity corresponds to the enzyme activity remaining when compared to the initial enzyme activity available before storage at time 0.

1. A liquid enzyme preparation comprising:
a. 0.5% to 15% by weight of at least one hydrolase (EC 3) and
b. 2% to 70% by weight of at least one compound according to formula (I) 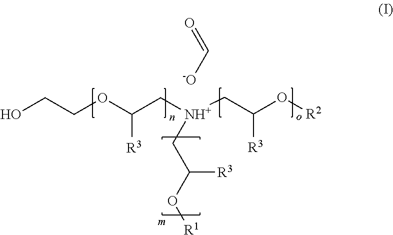
wherein R¹ and R² are selected from the group consisting of H and C₂H₄OH,
each of R³ is independently selected from the group consisting of H, methyl and ethyl,
and m, n, o are each individually 0-2;
wherein the amount of hydrolase refers to 100% active hydrolase.

2. The liquid enzyme preparation according to claim 1, wherein the liquid enzyme preparation comprises water in an amount not exceeding 15% by weight.

3. The liquid enzyme preparation according to claim 1, wherein the liquid enzyme preparation is essentially devoid of surface-active anionic compounds and complexing anionic compounds.

4. The liquid enzyme preparation according to claim 1, wherein the liquid enzyme preparation is essentially devoid of enzyme stabilizers selected from the group consisting of boron-containing compounds and peptide stabilizers.

5. The liquid enzyme preparation according to claim 1, wherein the liquid enzyme preparation further comprises at least one solvent selected from the group consisting of diols, triols, and sugar alcohols and/or further comprises at least one salt selected from the group consisting of a salt of a monovalent cation and a monovalent anion of 1-6 carbons, NaCl, KCl, CaCl2 and Na2SO4.

6. A method of preparing a liquid enzyme preparation comprising mixing at least one hydrolase (EC 3) with at least one compound according to formula (I) 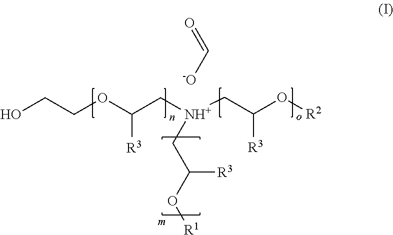
wherein R¹ and R² are selected from the group consisting of H and C₂H₄OH,
each of R³ is independently selected from the group consisting of H, methyl and ethyl,
and m, n, o are each individually 0-2.

7. The method according to claim 6, wherein the at least one hydrolase is comprised in a liquid enzyme concentrate prior to mixing with the at least one compound according to formula (I).

8. The method according to claim 6, wherein the at least one hydrolase is dissolved in a solvent selected from the group consisting of water and organic solvent prior to mixing with at least one compound according to formula (I).

9. A method of using at least one compound according to formula (I) 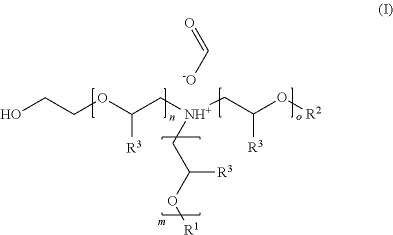
wherein R¹ and R² are selected from the group consisting of H and C₂H₄OH,
each of R³ is independently selected from the group consisting of H, methyl and ethyl,
and m, n, o are each individually 0-2;
the method comprising using the at least one compound to provide a liquid enzyme preparation, which is homogeneous in its appearance and increased in stability of at least one hydrolase when compared to a liquid enzyme preparation lacking the compound according to formula (I).

10. A liquid detergent formulation comprising:
(A) 0.0005% to 0.4% by weight of at least one hydrolase (EC 3);
(B) 4% to 20% by weight of a compound according to formula (I); 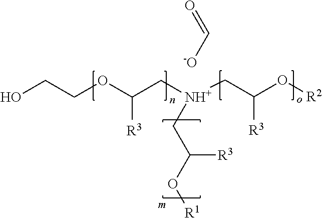
wherein R¹ and R² are selected from the group consisting of H and C₂H₄OH,
each of R³ is independently selected from H, methyl and ethyl,
and m, n, o are each individually 0-2; and
(C) at least 5% of at least one anionic compound.

11. The liquid detergent formulation according to claim 10, wherein the anionic compound is selected from the group consisting of a surface-active anionic compound and a complexing anionic compound.

12. The liquid detergent formulation according to claim 10, wherein the liquid detergent formulation comprises ≤3% by weight of sodium formate.

13. A method of preparing a liquid detergent formulation comprising mixing at least one hydrolase, at least one compound according to formula (I) 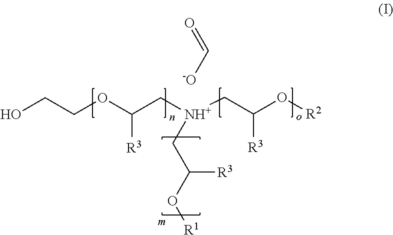
wherein R¹ and R² are selected from the group consisting of H and C₂H₄OH,
each of R³ is independently selected from the group consisting of H, methyl and ethyl,
and m, n, o are each individually 0-2;
and at least one anionic compound in one or more steps.

14. A method of using at least one compound according to formula (I) 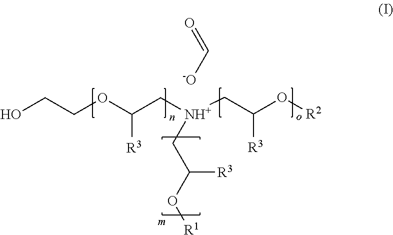
wherein R¹ and R² are selected from the group consisting of H and C₂H₄OH,
each of R³ is independently selected from the group consisting of H, methyl and ethyl,
and m, n, o are each individually 0-2;
the method comprising using the at least one compound to stabilize at least one hydrolase comprised in a liquid detergent formulation comprising at least one anionic compound selected from the group consisting of surface active anionic compounds and complexing anionic compounds or to provide a liquid detergent formulation, which is homogeneous in its appearance and with increased stability of at least one hydrolase when compared to a liquid detergent formulation lacking the compound according to formula (I).

15. A method to improve detergency of a liquid detergent formulation by adding at least one compound according to formula (I) 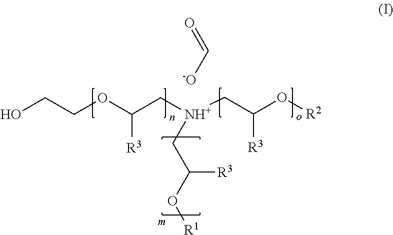
wherein R¹ and R² are selected from the group consisting of H and C₂H₄OH,
each of R³ is independently selected from the group consisting of H, methyl and ethyl,
and m, n, o are each individually 0-2;
to a hydrolase-containing liquid detergent formulation.

16. The liquid enzyme preparation according to claim 1, wherein the hydrolase is selected from the group consisting of proteases, amylases, lipases, cellulases, hemicellulase, mannanases, xylanases, DNases, dispersins, pectinases, and cutinases.

17. The liquid enzyme preparation according to claim 1, wherein the at least one compound according to formula (I) is triethanolamine formate.

18. The method according to claim 6, wherein the liquid enzyme concentrate originates from fermentative enzyme production.

19. The method according to claim 6, wherein the at least one hydrolase is dissolved in a solvent selected from the group consisting of water and organic solvent prior to mixing with at least one compound according to formula (I).

20. The liquid detergent formulation according to claim 10, wherein the surface-active anionic compound is selected from the group consisting of LAS or AES and wherein the complexing anionic compound is selected from the group consisting of citrates (NPB1) and aminocarboxylates (NPB3).
